(19) United States
(12) Patent Application Publication
(10) Pub. No.: US 2024/0058265 A1
(43) Pub. Date: Feb. 22, 2024

(54) TREATMENT OF OCULAR DISEASES USING ENDOTHELIN RECEPTOR ANTAGONISTS

(71) Applicant: Perfuse Therapeutics, Inc., San Francisco, CA (US)

(72) Inventors: Cheng-Wen Lin, Cary, NC (US); Angela Dawn Glendenning, Raleigh, NC (US); Sevgi Gurkan, Belmont, CA (US)

(21) Appl. No.: 18/494,545

(22) Filed: Oct. 25, 2023

Related U.S. Application Data
(63) Continuation of application No. PCT/US2022/027045, filed on Apr. 29, 2022.
(60) Provisional application No. 63/182,750, filed on Apr. 30, 2021.

(51) Int. Cl. A61K 9/00; A61K 47/34; A61K 31/422; A61P 27/06

(52) U.S. Cl. CPC A61K 9/0051; A61K 47/34; A61K 31/422; A61P 27/06

(57) ABSTRACT

The present disclosure relates to the discovery that certain diseases of the eye that profoundly affect the human visual system and, as a result, quality of life, may be treated using Edonentan or A-182086. Examples of the diseases include, but not limited to, ocular neovascularization, neovascular glaucoma, vascular leak, macular edema, and a neovascular age-related macular degeneration.

### RELATED APPLICATIONS

This application is a continuation of International Application Number PCT/US2022/027045 filed on Apr. 29, 2022, which claims priority to U.S. Provisional Patent Application No. 63/182,750, filed on Apr. 30, 2021, the entire contents of each of which are hereby incorporated by reference for all purposes.

### BACKGROUND

Examples of debilitating ocular diseases include neovascular glaucoma, ocular neovascularization, vascular leak, macular edema, neovascular age-related macular degeneration, retinal vein occlusion (RVO), and retinopathy of prematurity (ROP). These ocular diseases can variously cause long term damage to the eye and, ultimately, blindness. While neonates, the young, adults of all ages and the elderly are affected, only a handful of treatments exist. These treatments are only for a subset of ocular diseases and slow, but do not prevent, blindness. The annual economic burden on the U.S. alone is over $100 billion.

Ocular neovascularization, the formation of new vessels from the existing vascular tree, is a major cause of severe vision loss and significant visual impairment, worldwide. It can affect different structures in the eye, including the retina, choroid and cornea. It occurs when new abnormal blood vessels grow and spread throughout the retina and/or other parts of the eye (e.g. the tissue that lines the back of the eye, and the anterior chamber). The new abnormal blood vessels, in contrast to the normal blood vessels, are leaky and allow fluid from the blood to enter the retina. The fluid can immediately distort the vision and damage the retina.

Neovascular glaucoma (NVG) is a potentially blinding secondary glaucoma, characterized by the development of neovascularization of the iris, elevated intraocular pressure (TOP) and, in many instances, poor visual prognosis. NVG is a severe form of glaucoma attributed to new blood vessels obstructing aqueous humor outflow, secondary to posterior segment ischemia. It is associated with the development of a fibrovascular membrane on the anterior surface of the iris and iridocorneal angle of anterior chamber.

Retinal vein occlusion (RVO) is a vascular disorder of the retina and one of the most common causes of vision loss worldwide. Specifically, it is the second most common cause of blindness from retinal vascular disease after diabetic retinopathy. RVO is often the result of underlying health problems (e.g., high blood pressure, high cholesterol levels, diabetes, and other health problems). There are two types of retinal vein occlusion: central retinal vein occlusion (CRVO) is the blockage of the main retinal vein, and branch retinal vein occlusion (BRVO) is the blockage of one of the smaller branch veins.

Currently, there is no way to unblock retinal veins, and accepted treatments are directed to addressing health problems related to the retinal vein occlusion. Vision may come back in some eyes that have had a retinal vein occlusion. About ⅓ have some improvement, about ⅓ stay the same and about ⅓ gradually improve, but it can take a year or more to determine the final outcome. In some cases, the blocked vessels will lead to fluid accumulation in the retina. In other cases, occurrence of ischemia causes the formation of new blood vessels. RVO is currently treated with intravitreal injection of anti-vascular endothelial growth factor (VEGF) drugs.

Retinopathy of prematurity (ROP) can occur due to premature birth. Abnormal, leaky blood vessel growth (neovascularization) in the retina occurs secondary to other treatments for prematurity and can often lead to neonatal blindness. During pregnancy, blood vessels grow from the center of a developing baby's retina 16 weeks into the mother's pregnancy, and then branch outward and reach the edges of the retina 8 months into the pregnancy. In babies born prematurely, normal retinal vessel growth is incomplete and may therefore be more readily disrupted.

Accordingly, there is an unmet need to more effectively reduce the incidence of, treat or otherwise ameliorate neovascular glaucoma, ocular neovascularization, vascular leak, macular edema, neovascular age-related macular degeneration, retinal vein occlusion (RVO), and retinopathy of prematurity (ROP).

### SUMMARY

The present disclosure provides a method of preventing, treating, or ameliorating an ocular disease in a subject in need thereof, comprising contacting an optical tissue in a subject with a composition comprising a therapeutically effective amount of an endothelin receptor antagonist or a pharmaceutically acceptable salt thereof. The ocular disease that can be treated with using the methods described herein include, but not limited to, neovascular glaucoma, retinal vein occlusion (RVO), retinopathy of prematurity (ROP), an ocular neovascularization, a vascular leakage, a neovascular age-related macular degeneration, and macular edema.

The method comprises contacting an optical tissue in a subject with a composition comprising a therapeutically effective amount of endothelin receptor antagonist, or a pharmaceutically acceptable salt thereof. In various embodiments, the endothelin receptor antagonist is selected from the group consisting of Edonentan, Tezosentan, A-182086, Clazosentan, S1255, ACT-132577, Enrasentan, and Sparsentan. Preferably, the endothelin receptor antagonist is Edonentan or A-182086.

The disclosure also provides a method of preventing, treating, or ameliorating an ocular neovascularization, a vascular leakage, macular edema, or a neovascular age-related macular degeneration in a subject in need thereof, comprising contacting an optical tissue in a subject with a composition comprising a therapeutically effective amount of a compound of Formula I:

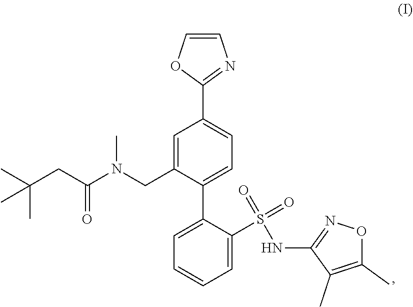 [SMILES: Cc1onc(NS(=O)(=O)c2ccccc2-c2ccc(-c3ncco3)cc2CN(C)C(=O)CC(C)(C)C)c1C]

or pharmaceutically acceptable salt thereof.

### BRIEF DESCRIPTION OF THE DRAWINGS

FIG. 1 depicts optical coherence tomography—angiography (OCT-A) images of a representative experiment revealing severe vasospasm in the rabbit retinal vascular structure in focus 45 min after 0.5 μg of Endothelin-1 (ET-1) administration via intravitreal injection (IVT) injection.

FIG. 2 depicts fluorescein angiography (FA) images revealing reversal of ET-1 induced vasospasm after IVT administration of 10 μg Edonentan.

FIG. 3 depicts the comparison of fluorescein dye velocity as an index of retinal blood flow in healthy rabbits (n=5/group) after IVT administration of vehicle alone (control group), or 0.5 μg of ET-1 alone, or 0.5 μg of ET-1 and 10 μg of Edonentan, or 0.5 of ET-1 and 10 μg of A-182086 — revealing prolongation of dye velocity/reduction of flow in ET-1 treated rabbits which is improved to control levels following treatment with Edonentan or A-182086.

FIG. 4 depicts the comparison of neovascular area (NV) in 7-day old neonatal C57BL/6 mice with oxygen-induced ischemic retinopathy (OIR) after topical eyedrops of Edonentan, vehicle control, or intraperitoneal injections with aflibercept at 1 mg/kg.

FIG. 5A depicts the comparison of retinal ganglion cell (RGC) counts in the peripheral retina of rats with elevated intraocular pressure (TOP) (n=4 rats/group for control, n=6 rats/group for Edonentan) after topical administration of vehicle alone (control group) or Edonentan. FIG. 5B depicts the comparison of pattern electroretinogram (PERG) changes in with elevated intraocular pressure (TOP) rats (n=4 rats/group for control, n=5 rats/group for Edonentan) after topical administration of vehicle alone (control group) or Edonentan. FIG. 5A and FIG. 5B reveal prevented RGC loss and maintained RGC function after treatment with Edonentan. FIG. 5C depicts pharmacokinetic profiles of topically or orally administered Edonentan in the plasma, retina/retinal pigment epithelium (RPE)/choroid, vitreous humor and aqueous humor of rats. FIG. 5C reveals the ability of Edonentan to permeate through cornea/sclera and achieve retina exposure after topical administration.

FIG. 6A depicts the comparison of retinal ganglion cell (RGC) counts in the peripheral retina of rats (n=4 rats/group for control, n=6 rats/group for A-182086) with elevated intraocular pressure (TOP) after topical administration of vehicle alone (control group) or A-182086. FIG. 6B depicts the comparison of pattern electroretinogram (PERG) changes in rats (n=4 rats/group for control, n=5 rats/group for A-182086) with elevated intraocular pressure (TOP) after topical administration of vehicle alone (control group) or A-182086. FIG. 6A and FIG. 6B reveal prevented RGC loss and maintained RGC function after treatment with A-182086.

FIG. 6C depicts pharmacokinetic profiles of topically or orally administered A-182086 in the plasma, retina/retinal pigment epithelium (RPE)/choroid, vitreous humor and aqueous humor of rats. FIG. 6C reveals the ability of A-182086 to permeate through cornea/sclera and achieve retina exposure after topical administration.

FIGS. 7A-7L depict laser speckle flow graphs (LSFG) for the comparison of an experimental glaucoma eye and a contralateral healthy eye (control) of three non-human primates in global average mean blur rate (MBR) or MBR change from baseline over time as an index of optic nerve head (ONH) blood flow in a laser-induced glaucoma model. FIG. 7M shows the aggregate results from the three non-human primates. FIG. 7N shows an LSFG scan of one of the non-human primates at various selected time points.

FIG. 8A and FIG. 8B depict the comparison of fluorescein dye velocity as an index of retinal blood flow in ET-1 induced rabbits (n=5 rabbits/group) after IVT administration of vehicle alone (control), 0.1 μg of ET-1 and 10 μg of Edonentan, 0.1 μg of ET-1 and 2.5 μg of Edonentan, 0.1 μg of ET-1 and 0.5 μg of Edonentan, 0.1 μg of ET-1 and 0.1 μg of Edonentan, or 0.1 μg of ET-1 alone—revealing dose-response in the rabbit ET-1 induced vasospasm model.

FIG. 9A, FIG. 9B, FIG. 9C and FIG. 9D depict pharmacokinetic profiles of intravitreally delivered Edonentan in the plasma, retina, iris-ciliary body (ICB), retinal pigment epithelium (RPE)/choroid, vitreous humor or aqueous humor of rabbits (FIG. 9A, FIG. 9B, FIG. 9C and FIG. 9D)—revealing longer t_{1/2} for Edonentan.

FIG. 10 depicts pharmacokinetic profiles of topically administered Edonentan in the plasma, retina, vitreous humor and bulbar conjunctiva of rabbits—revealing the ability of Edonentan to penetrate through ocular layers after a single topical application to the eye.

FIG. 11A and FIG. 11B depict pharmacokinetic profiles of intravitreally delivered Edonentan in the retina and retinal pigment epithelium (RPE)/choroid in rabbits (FIG. 11A, FIG. 11B) dosed with 2 implants of injection molded and ram extruded product.

FIG. 12 depicts an exemplary overlay of XRPD pattern of Forms 1-4.

FIG. 13 depicts an exemplary XRPD pattern of Form 1.

FIG. 14 depicts an exemplary XRPD pattern of Form 2.

FIG. 15 depicts an exemplary XRPD pattern of Form 3.

FIG. 16 depicts an exemplary XRPD pattern of Form 4.

FIG. 17 depicts an exemplary DSC curve of Form 1.

FIG. 18 depicts an exemplary DSC curve of Form 2.

FIG. 19 depicts an exemplary DSC curve of Form 3.

FIG. 20 depicts an exemplary DSC curve of Form 4.

FIG. 21 depicts XRPD characteristic peaks for crystalline Form 4 shown in FIG. 16.

FIG. 22 depicts a time course of Edonentan retina levels during 12-week single dose intravitreal ocular pharmacokinetic study in pigmented rabbits dosed with 2 implants of injection molded product.

FIG. 23 depicts a time course of Edonentan RPE/choroid levels during 12-week single dose intravitreal ocular pharmacokinetic study in pigmented rabbits dosed with 2 implants of injection molded product.

### DETAILED DESCRIPTION

The present disclosure provides methods for preventing, treating, or ameliorating an ocular neovascularization in a subject in need thereof. Also provided herein are method for preventing, treating, or ameliorating a vascular leakage, or a neovascular age-related macular degeneration in a subject in need thereof. The disclosure arises from the discovery that Edonentan and A-182086 can be used to prevent, treat or otherwise ameliorate ocular diseases including, but not limited to, neovascular glaucoma, retinal vein occlusion (RVO), and retinopathy of prematurity (ROP).

### Compounds

Methods of the present invention include contacting the eye tissue or administration (e.g. via topically, intra-ocularly, intravitreally) of a therapeutically effective amount of a compound described herein, or a pharmaceutically acceptable salt thereof. The compounds contemplated herein are endothelin receptor antagonists such as Edonentan, Tezosentan, A-182086, Clazosentan, S1255, ACT-132577, Enrasentan, and Sparsentan.

In certain embodiments, the compound is a compound of Formula I:

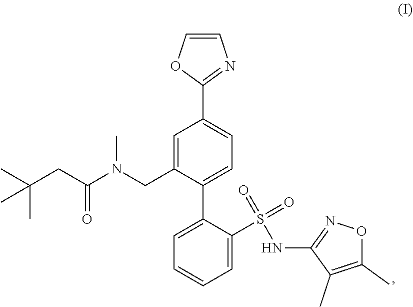 [SMILES: Cc1onc(NS(=O)(=O)c2ccccc2-c2ccc(-c3ncco3)cc2CN(C)C(=O)CC(C)(C)C)c1C]

or a pharmaceutically acceptable salt thereof.

The compound of Formula I is also known as Edonentan. Edonentan has the chemical name of N-[[2′-[[(4,5-dimethyl-3-isoxazolyl)amino]sulfonyl]-4-(2-oxazolyl)[1,1′-biphenyl]-2-yl]methyl]-N,3,3-trimethylbutanamide (molecular weight of 536.6 g/mol). Methods of preparing Edonentan are well known to a person of skill in the art. Suitable methods are disclosed, for example, in U.S. Pat. No. 6,043,265. Edonentan is a highly selective and very potent endothelin A receptor antagonist. Edonentan was developed as a second-generation analog following the discontinuation of the first clinical candidate, BMS-193884, which was being developed for the treatment of congestive heart failure (CHF). Edonentan was in phase I trials by April 2002, but its development was discontinued.

In some embodiments, the composition described herein comprises A-182086, which has the structure:

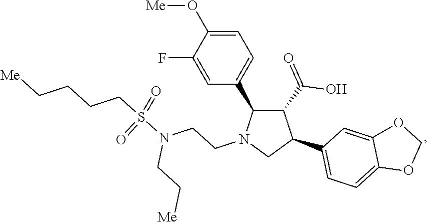 [SMILES: CCCCCS(=O)(=O)N(CCC)CCN1C[C@H](c2ccc3c(c2)OCO3)[C@@H](C(=O)O)[C@@H]1c1ccc(OC)c(F)c1]

or a pharmaceutically acceptable salt thereof.

A-182086 has the chemical name of (2R,3R,4S)-4-(2H-1,3-benzodioxol-5-yl)-2-(3-fluoro-4-methoxyphenyl)-1-[2-(N-propylpentane-1-sulfonamido)ethyl]pyrrolidine-3-carboxylic acid (molecular weight of 578.7 g/mol). Methods of preparing A-182086 are well known to a person of skill in the art. Suitable methods are disclosed, for example, in U.S. Pat. No. 6,162,927. A-182086 is a potent dual ETA/ETB receptor antagonist with 4-fold ETA/ETB selectivity. A-182086 has not been studied in a clinical setting to date.

As described herein, the disclosure provides a method of preventing, treating, or ameliorating an ocular neovascularization in a subject in need thereof, comprising contacting an optical tissue in a subject with a composition comprising a therapeutically effective amount of a compound of Formula I or A-182086.

Also provided herein is a method of preventing, treating, or ameliorating a vascular leakage in a subject in need thereof, comprising contacting an optical tissue in a subject with a composition comprising a therapeutically effective amount of a compound of Formula I or A-182086.

The disclosure also provides a method of preventing, treating, or ameliorating a neovascular age-related macular degeneration in a subject in need thereof, comprising contacting an optical tissue in a subject with a composition comprising a therapeutically effective amount of a compound of Formula I or A-182086.

Yet also provided herein is a method of preventing, treating, or ameliorating a macular edema in a subject in need thereof, comprising contacting an optical tissue in a subject with a composition comprising a therapeutically effective amount of a compound of Formula I or A-182086.

### Crystalline Forms

Methods of the present invention include contacting the eye tissue or administration (e.g. via topically, intra-ocularly, intravitreally) of a solid form of a compound of Formula I. In certain embodiments, the solid form of the compound of Formula I:

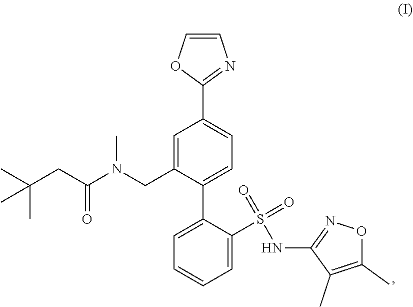 [SMILES: Cc1onc(NS(=O)(=O)c2ccccc2-c2ccc(-c3ncco3)cc2CN(C)C(=O)CC(C)(C)C)c1C]

is an anhydrous crystalline form (Form 4), having an X-ray powder diffraction pattern comprising at least three characterization peaks, in terms of 2θ, selected from peaks at 5.6±0.2°, 11.4±0.2°, 17.7±0.2°, 19.3±0.2°, 21.1±0.2°, and 21.9±0.2°.

In some embodiments of the solid form, the anhydrous crystalline Form 4 has the following X-ray powder diffraction pattern expressed in terms of diffraction angles (2θ): 5.6±0.2°, 11.4±0.2°, 17.7±0.2°, 19.3±0.2°, and 21.9±0.2°. In some embodiments of the solid form, the anhydrous crystalline Form 4 has the following X-ray powder diffraction pattern expressed in terms of diffraction angles (2θ): 11.4±0.2°, 17.7±0.2°, and 19.3±0.2°. In some embodiments of the solid form, the anhydrous crystalline Form 4 shows a Tₘ of about 163° C. by DSC analysis. In some embodiments of the solid form, the anhydrous crystalline Form 4 has the following X-ray powder diffraction pattern expressed in terms of diffraction angles (2θ): 5.6±0.2°, 11.4±0.2°, 17.7±0.2°, 19.3±0.2°, and 21.9±0.2°. In some embodiments of the solid form, the anhydrous crystalline Form 4 has the following X-ray powder diffraction pattern expressed in terms of diffraction angles (2θ): 11.4±0.2°, 17.7±0.2°, and 19.3±0.2°. In some embodiments of the solid form, the anhydrous crystalline Form 4 shows a T. of about 163° C. by DSC analysis.

In some embodiments, said compound is 90% by weight or more in crystalline Form 4 based on the total weight of the compound present in the composition. In some embodiments, the compound of Formula I is 95% by weight or more in crystalline Form 4 based on the total weight of the compound present in the composition. In some embodiments, the compound of Formula I is 96% by weight or more in crystalline Form 4 based on the total weight of the compound present in the composition. In some embodiments, the compound of Formula I is 97% by weight or more in crystalline Form 4 based on the total weight of the compound present in the composition. In some embodiments, the compound of Formula I is 98% by weight or more in crystalline Form 4 based on the total weight of the compound present in the composition. In some embodiments, the compound of Formula I is 99% by weight or more in crystalline Form 4 based on the total weight of the compound present in the composition.

In certain embodiments, the compound of Formula I is an anhydrous crystalline form (Form 1), wherein the anhydrous crystalline Form 1 has an X-ray powder diffraction pattern comprising at least three characterization peaks, in terms of 2θ, selected from peaks at 6.3±0.2°, 7.5±0.2°, 11.7±0.2°, 15.1±0.2°, and 17.3±0.2°; and said compound is 90% by weight or more in crystalline Form 1 based on the total weight of the compound present in the composition.

In certain embodiments, the compound of Formula I is a monohydrate crystalline form (Form 2), wherein the monohydrate crystalline Form 2 has an X-ray powder diffraction pattern comprising at least three characterization peaks, in terms of 2θ, selected from peaks at 9.6±0.2°, 10.4±0.2°, 19.6±0.2°, 19.7±0.2°, 22.0±0.2°, 22.9±0.2°, and 23.7±0.2°; and said compound is 90% by weight or more in crystalline Form 2 based on the total weight of the compound present in the composition;

In certain embodiments, the compound of Formula I is an anhydrous crystalline (Form 3), wherein the anhydrous crystalline Form 3 has an X-ray powder diffraction pattern comprising at least three characterization peaks, in terms of 2θ, selected from peaks at 7.8±0.2°, 9.0±0.2°, 11.6±0.2°, 15.8±0.2°, and 19.1±0.2′; and said compound is 90% by weight or more in crystalline Form 3 based on the total weight of the compound present in the composition.

As used herein, the term “amorphous” refers to a solid material having no long range order in the position of its molecules. Amorphous solids are generally supercooled liquids in which the molecules are arranged in a random manner so that there is no well-defined arrangement, e.g., molecular packing, and no long range order. Amorphous solids are generally isotropic, i.e. exhibit similar properties in all directions and do not have definite melting points. For example, an amorphous material is a solid material having no sharp characteristic crystalline peak(s) in its X-ray power diffraction (XRPD) pattern (i.e., is not crystalline as determined by XRPD). Instead, one or several broad peaks (e.g., halos) appear in its XRPD pattern.

Hydrate forms of crystalline Edonentan are contemplated, e.g., Edonentan (H₂O)ₘ, where m is a fractional or whole number between about 0 and about 4 inclusive. For example, contemplated herein are anhydrate or monohydrate forms of crystalline Edonentan. In an embodiment, a disclosed crystalline form of Edonentan may have a water level of about 1 to 10% by weight (e.g., 3 to 9% or 5 to 8% by weight).

### Biodegradable Ocular Implant

Methods of the present invention include contacting the eye tissue or administration (e.g. via topically, intra-ocularly, intravitreally) of a biodegradable ocular implants comprising a compound of Formula I (also referred to herein as Edonentan).

The biodegradable ocular implants comprising Edonentan described herein can be used for preventing, treating, or ameliorating an ocular neovascularization, a vascular leakage, a neovascular age-related macular degeneration, a neovascular age-related macular degeneration, or macular edema in a subject in need thereof.

The biodegradable ocular implant described herein comprises a biodegradable polymer containing a compound incorporated therein. In preferrable embodiments, the compound is a compound of Formula I.

In various embodiments, the implant has a diameter of about 300 μm to about 400 μm (e.g., about 300 μm, about 325 μm, about 350 μm, about 375 μm, and about 400 μm), and a length of about 4 mm to about 5 mm (e.g., about 4.1 mm, about 4.2 mm, about 4.3 mm, about 4.4 mm, about 4.5 mm, about 4.6 mm, about 4.7 mm, about 4.8 mm, about 4.9 mm, and about 5 mm). In certain embodiments, the implant has a diameter of about 300 μm and a length of about 4 mm. In certain embodiments, the implant has a diameter of about 340 μm and a length of about 4 mm.

In various embodiments, the implant has a total weight of about 250 μg to about 450 μg (e.g., about 250 μg, about 270 μg, about 290 μg, about 310 μg, about 330 μg, about 350 μg, about 370 μg, about 390 μg, about 410 μg, about 430 μg, and about 450 μg). In various embodiments, the implant has a total weight of about 300 μg to about 450 μg. In various embodiments, the implant has a total weight of about 350 μg to about 450 μg. In some embodiments, the implant has a total weight of about 380 μg.

In various embodiments, the concentration of the compound (e.g., compound of Formula I) in the biodegradable ocular implant is present in the biodegradable polymer is about 5% w/w to about 95% w/w (e.g., about 10% w/w to about 95% w/w, about 15% w/w to about 95% w/w, about 20% w/w to about 95% w/w, about 25% w/w to about 95% w/w, about 30% w/w to about 95% w/w, about 35% w/w to about 95% w/w, about 40% w/w to about 95% w/w, about 45% w/w to about 95% w/w, about 50% w/w to about 95% w/w, about 55% w/w to about 95% w/w, about 60% w/w to about 95% w/w, about 65% w/w to about 95% w/w, about 70% w/w to about 95% w/w, about 75% w/w to about 95% w/w, about 80% w/w to about 95% w/w, about 85% w/w, about 95% w/w, about 90% w/w to about 95% w/w, about 5% w/w to about 10% w/w, about 5% w/w to about 15% w/w, about 5% w/w to about 20% w/w, about 5% w/w to about 25% w/w, about 5% w/w to about 30% w/w, about 5% w/w to about 35% w/w, about 5% w/w to about 40% w/w, about 5% w/w to about 45% w/w, about 5% w/w to about 50% w/w, about 5% w/w to about 55% w/w, about 5% w/w to about 60% w/w, about 5% w/w to about 65% w/w, about 5% w/w to about 70% w/w, about 5% w/w to about 75% w/w, about 5% w/w to about 80% w/w, about 5% w/w to about 85% w/w, and about 5% w/w to about 90% w/w). In certain embodiments, the concentration of the compound in the biodegradable ocular implant is present in the biodegradable polymer is about 20% w/w to about 60% w/w (e.g., about 20% w/w to about 55% w/w, about 20% w/w to about 50% w/w, about 20% w/w to about 45% w/w, about 20% w/w to about 40% w/w, about 20% w/w to about 35% w/w, about 20% w/w to about 30% w/w, about 20% w/w to about 25% w/w, about 25% w/w to about 60% w/w, about 30% w/w to about 60% w/w, about 35% w/w to about 60% w/w, about 40% w/w to about 60% w/w, about 45% w/w to about 60% w/w, about 50% w/w to about 60% w/w, about 55% w/w to about 60% w/w). In certain embodiments, the concentration of the compound in the biodegradable ocular implant is present in the biodegradable polymer is about 25% w/w to about 45% w/w. In certain embodiments, the concentration of the compound in the biodegradable ocular implant is present in the biodegradable polymer is about 40% w/w to about 50% w/w (e.g., about 40% w/w to about 45% w/w, about 45% w/w to about 50% w/w). In various embodiments, the concentration of the compound is about 5% w/w, about 10% w/w, about 15% w/w, about 20% w/w, about 25% w/w, about 30% w/w, about 35% w/w, about 40% w/w, about 45% w/w, or about 50% w/w. In various embodiments, the concentration of the compound is about 30% w/w. In various embodiments, the concentration of the compound is about 40% w/w. In various embodiments, the concentration of the compound is about 45% w/w. In various embodiments, the concentration of the compound is about 50% w/w.

In embodiments, the amount of the compound (e.g., compound of Formula I, A-182086) in the biodegradable ocular implant is present in the biodegradable polymer is about 1 μg to about 500 μg (e.g., about 10 μg to about 500 μg, about 20 μg to about 500 μg, about 30 μg to about 500 μg, about 40 μg to about 500 μg, about 50 μg to about 500 μg, about 60 μg to about 500 μg, about 70 μg to about 500 μg, about 80 μg to about 500 μg, about 90 μg to about 500 μg, about 100 μg to about 500 μg, about 100 μg to about 500 μg, about 125 μg to about 500 μg, about 150 μg to about 500 μg, about 175 μg to about 500 μg, about 200 μg to about 500 μg, about 225 μg to about 500 μg, about 250 μg to about 500 μg, about 275 μg to about 500 μg, about 300 μg to about 500 μg, about 325 μg to about 500 μg, about 350 μg to about 500 μg, about 375 μg to about 500 μg, about 400 μg to about 500 μg, about 425 μg to about 500 μg, about 450 μg to about 500 μg, and about 475 μg to about 500 μg). In various embodiments, the amount of the compound (e.g., compound of Formula I, A-182086) in the biodegradable ocular implant is present in the biodegradable polymer is about 70 μg to about 230 μg (e.g., about 70 μg, about 75 μg, about 80 μg, about 85 μg, about 90 μg, about 95 μg, about 100 μg, about 105 μg, about 110 μg, about 115 μg, about 120 μg, about 125 μg, about 130 μg, about 135 μg, about 140 μg, about 145 μg, about 150 μg, about 155 μg, about 160 μg, about 165 μg, about 170 μg, about 175 μg, about 180 μg, about 185 μg, about 190 μg, about 195 μg, about 200 μg, about 205 μg, about 210 μg, about 215 μg, about 220 μg, about 225 μg, and about 230 μg). In various embodiments, the amount of the compound (e.g., compound of Formula I, A-182086) in the biodegradable ocular implant is present in the biodegradable polymer is about 165 μg to about 220 μg (e.g., about 165 μg, about 170 μg, about 175 μg, about 180 μg, about 185 μg, about 190 μg, about 195 μg, about 200 μg, about 205 μg, about 210 μg, about 215 μg, and about 220 μg). In some embodiments, the amount of the compound (e.g., compound of Formula I, A-182086) in the biodegradable ocular implant is present in the biodegradable polymer is about 150 μg to about 250 μg, about 300 μg to about 550 μg, or about 300 μg to about 600 μg. In various embodiments, the amount of the compound (e.g., compound of Formula I, A-182086) in the biodegradable ocular implant is present in the biodegradable polymer is about 330 μg to about 500 μg (e.g., about 330 μg, about 335 μg, about 340 μg, about 345 μg, about 350 μg, about 355 μg, about 360 μg, about 365 μg, about 370 μg, about 375 μg, about 380 μg, about 385 μg, about 390 μg, about 395 μg, about 400 μg, about 405 μg, about 410 μg, about 415 μg, about 420 μg, about 425 μg, about 430 μg, about 435 μg, about 440 μg, about 445 μg, about 450 μg, about 455 μg, about 460 μg, about 465 μg, about 470 μg, about 475 μg, about 480 μg, about 485 μg, about 490 μg, about 495 μg, and about 500 μg).

In some embodiments, the biodegradable ocular implant comprises initially at least about 95% to about 99% (e.g., about 95%, about 96%, about 97%, about 98%, and about 99%) of a matrix of the biodegradable polymer and the compound. In some embodiments, the biodegradable ocular implant comprises initially at least 95% of a matrix of the biodegradable polymer and the compound. In some embodiments, the biodegradable ocular implant comprises initially at least about 80% to about 95% (e.g., about 80%, about 81%, about 82%, about 83%, about 84%, about 85%, about 86%, about 87%, about 88%, about 89%, about 90%, about 91%, about 92%, about 93%, about 94%, and about 95%) of a matrix of the biodegradable polymer and the compound.

The rate of therapeutic agent (e.g., a compound of Formula I) release from an intravitreal implant or particle suspension (for example, a biodegradable ocular implant of the present disclosure) may depend on several factors, including but not limited to the surface area of the implant, therapeutic agent content, and water solubility of the therapeutic agent, and speed of polymer degradation.

In some embodiments, less than 40% (e.g., about 40%, about 35%, about 30%, about 25%, about 20%, about 15%, about 10%, and about 5%) of the compound is released from the biodegradable ocular implant when placed in phosphate buffered saline (PBS) in about 1 month. In some embodiments, less than 90% (e.g., about 90%, about 85%, about 80%, about 75%, about 70%, about 65%, about 60%, about 55%, about 50%, about 45%, about 40%, about 35%, about 30%, about 25%, about 20%, about 15%, about 10%, and about 5%) of the compound is released from the biodegradable ocular implant when placed in phosphate buffered saline (PBS) in about 1 month to about 12 months (about 1 month, about 2 months, about 3 months, about 4 months, about 5 months, about 6 months, about 7 months, about 8 months, about 9 months, about 10 months, about 11 months, about 12 months).

In various embodiments, the implant is administered as an intravitreal administration. An intravitreal administration refers to drug administration into the vitreous humor of the eye. In some embodiments, the implant is administered locally to the back of the eye. In some embodiments, the implant is injected into the intravitreal space using a needle and applicator. In some embodiments, the biodegradable ocular implant comprises a dose of the compound (e.g., compound of Formula I or a crystalline form thereof) in a range of about 1 μg to about 1 mg (e.g., about 1 μg, about 10 μg, about 25 μg, about 50 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, about 500 μg, about 525 μg, about 550 μg, about 575 μg, about 600 μg, about 625 μg, about 650 μg, about 675 μg, about 700 μg, about 725 μg, about 750 μg, about 775 μg, about 800 μg, about 825 μg, about 850 μg, about 875 μg, about 900 μg, about 925 μg, about 950 μg, and about 975 μg). In some embodiments, the biodegradable ocular implant comprises a dose of the compound (e.g., compound of Formula I or a crystalline form thereof) in a range of about 10 μg to about 100 μg. In some embodiments, the biodegradable ocular implant comprises a dose of the compound (e.g., compound of Formula I or a crystalline form thereof) in a range of about 500 μg to about 4 mg (e.g., about 1 mg, about 1.5 mg, about 2 mg, about 2.5 mg, about 3 mg, and about 3.5 mg). In some embodiments, the dose is about 150 μg to about 250 μg. In certain embodiments, the dose is about 165 μg to about 220 μg (e.g., about 165 μg, about 170 μg, about 175 μg, about 180 μg, about 185 μg, about 190 μg, about 195 μg, about 200 μg, about 205 μg, about 210 μg, about 215 μg, and about 220 μg). In some embodiments, the dose is about 300 μg to about 500 μg. In some embodiments, the dose is about 300 μg to about 550 μg. In some embodiments, the dose is about 300 μg to about 600 μg. In certain embodiments, the dose is about 330 μg to about 500 μg (e.g., about 330 μg, about 335 μg, about 340 μg, about 345 μg, about 350 μg, about 355 μg, about 360 μg, about 365 μg, about 370 μg, about 375 μg, about 380 μg, about 385 μg, about 390 μg, about 395 μg, about 400 μg, about 405 μg, about 410 μg, about 415 μg, about 420 μg, about 425 μg, about 430 μg, about 435 μg, about 440 μg, about 445 μg, about 450 μg, about 455 μg, about 460 μg, about 465 μg, about 470 μg, about 475 μg, about 480 μg, about 485 μg, about 490 μg, about 495 μg, and about 500 μg). In some embodiments, the dose is about 200 μg to about 400 μg (e.g., about 200 μg, about 210 μg, about 220 μg, about 230 μg, about 240 μg, about 250 μg, about 260 μg, about 270 μg, about 280 μg, about 290 μg, about 300 μg, about 310 μg, about 320 μg, about 330 μg, about 340 μg, about 350 μg, about 360 μg, about 370 μg, about 380 μg, about 390 μg, about 400 μg). In some embodiments, the dose is about 175 μg.

In some embodiments, the biodegradable ocular implant is a sterile biodegradable ocular implant. As used herein, “sterile” refers to the composition meeting the requirements of sterility enforced by medicine regulatory authorities, such as the MCA in the UK or the FDA in the US. Tests are included in current versions of the compendia, such as the British Pharmacopoeia and the US Pharmacopoeia. In some embodiments, the biodegradable ocular implant is a substantially pure biodegradable ocular implant. In some embodiments, the biodegradable ocular implant is a medical-grade biodegradable ocular implant. In some embodiments, the biodegradable ocular implant is administered into the intravitreal space every 3 to 12 months.

### Biodegradable Polymers

Suitable polymeric materials or compositions for use in the implants described herein include those materials which are compatible, that is biocompatible, with the eye so as to cause no substantial interference with the functioning or physiology of the eye. Such polymeric materials may be biodegradable, bioerodible or both biodegradable and bioerodible.

The term “biodegrade” or “biodegradable” as used herein generally refers to a biologically assisted degradation process that the polymer making-up the implant undergoes in a biological environment, such as within the body of a subject. It would be appreciated that biodegradation encompasses within its scope the processes of absorption, dissolution, breaking down, degradation, assimilation, or otherwise removal of the implant from the body, a biological environment.

The term “polymer” as used herein encompasses both homopolymers (polymers having only one type of repeating unit) and copolymers (a polymer having more than one type of repeating unit).

The term “biodegradable polymer” as used herein refers to a polymer or polymers, which degrade in vivo, under physiological conditions. The release of the therapeutic agent occurs concurrent with, or subsequent to, the degradation of a biodegradable polymer over time.

In preferable embodiments, the biodegradable polymer is a PLGA (poly(lactic-co-glycolic acid)). PLGA polymers are known to degrade via backbone hydrolysis (bulk erosion) and the final degradation products are lactic and glycolic acids, which are non-toxic and considered natural metabolic compounds. Lactic and glycolic acids are eliminated safely via the Krebs cycle by conversion to carbon dioxide and water.

PLGA is synthesized through random ring-opening co-polymerization of the cyclic dimers of glycolic acid and lactic acid. Successive monomeric units of glycolic or lactic acid are linked together by ester linkages. The ratio of lactide to glycolide can be varied, altering the biodegradation characteristics of the product. By altering the ratio, it is possible to tailor the polymer degradation time. Importantly, drug release characteristics are affected by the rate of biodegradation, molecular weight, and degree of crystallinity in drug delivery systems. By altering and customizing the biodegradable polymer matrix, the drug delivery profile can be changed.

PLGA is cleaved predominantly by non-enzymatic hydrolysis of its ester linkages throughout the polymer matrix, in the presence of water in the surrounding tissues. PLGA polymers are biocompatible, because they undergo hydrolysis in the body to produce the original monomers, lactic acid and/or glycolic acid. Lactic and glycolic acids are nontoxic and eliminated safely via the Krebs cycle by conversion to carbon dioxide and water. The biocompatibility of PLGA polymers have been further examined in both non-ocular and ocular tissues of animals and humans. The findings indicate that the polymers are well tolerated.

Examples of PLGA polymers, which may be utilized in an embodiment of the disclosure, include the RESOMER® Product line from Evonik Industries identified as, but are not limited to, RG502, RG502H, RG503, RG503H, RG504, RG504H, RG505, RG653H, RG750S, RG752H, RG752S, RG753H, RG753S, RG755S, RG756S, RG757S, and RG858S.

Such PLGA polymers include both acid and ester terminated polymers with inherent viscosities ranging from approximately 0.14 to approximately 1.7 dL/g when measured at 0.1% w/v in CHCl₃ at 25° C. with an Ubbelhode size 0 c glass capillary viscometer. Example polymers used in various embodiments of the disclosure may include variation in the mole ratio of D,L-lactide to glycolide from approximately 50:50 to approximately 85:15, including, but not limited to, 50:50, 65:35, 75:25, and 85:15.

Other examples of PLGA polymers which may be utilized in an embodiment of the disclosure include those produced by Lakeshore Biomaterials identified as, but are not limited to, DLG 1A, DLG 3 A, or DLG 4A. Such DLG polymers include both acid (A) and ester (E) terminated polymers with inherent viscosities ranging from approximately 0.0.5 to approximately 1.0 dL/g when measured at 0.1% w/v in CHCl₃ at 25° C. with an Ubbelhode size 0 c glass capillary viscometer. Example polymers used in various embodiments of the disclosure may include variation in the mole ratio of D,L-lactide to glycolide from approximately 1:99 to approximately 99:1, including, but not limited to, 50:50, 65:35, 75:25, and 85:15.

RESOMERS® identified by an “RG” or “DLG” in the product name, such as RG752S, is a poly(D,L-lactide-co-glycolide) or PLGA having the general structure (V):

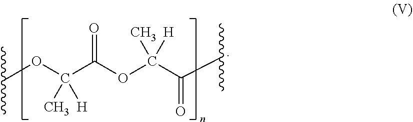

The synthesis of various molecular weights of DLG with various D,L-lactide-glycolide ratios is possible. In one embodiment, DLG, such as 1A, with an inherent viscosity of approximately 0.05 to approximately 0.15 dL/g can be used. In another embodiment, DLG, such as 2A, with an inherent viscosity of approximately 0.15 to approximately 0.25 dL/g can be used. [0168] Poly(D,L-lactide-co-glycolide) or PLGA copolymers can be synthesized at different ratios of lactide to glycolide, such as a lactide: glycolide ratio of 75:25. These copolymers can be an ester-terminated PLGA copolymer, as identified by the terminal “S” in the product name, or an acid-terminated PLGA copolymer, as identified by the terminal “H” in the product name.

In some embodiments, the biodegradable ocular implant of the disclosure comprises at least one PLGA, wherein each PLGA is independently selected from the group consisting of RG502, RG502S, RG502H, RG503, RG503H, RG504, RG504H, RG505, RG506, RG653H, RG752H, RG752S, RG753H, RG753S, RG755, RG755S, RG756, RG756S, RG757S, RG750S, RG858, and RG858S. In some embodiments, the biodegradable polymer comprises a poly(lactic-co-glycolic acid) (PLGA), wherein the PLGA is selected from the group consisting of RG502, RG503H, RG503, RG752S, RG753S, RG755S, RG756S, and RG858S. In some embodiments, the biodegradable polymer comprises a poly(lactic-co-glycolic acid) (PLGA), wherein the PLGA is selected from the group consisting of RG502, RG503, RG752S, RG753S, RG755S, RG756S, and RG858S. In some embodiments, the biodegradable ocular implant of the disclosure comprises one PLGA. In some embodiments, the PLGA has a ratio of PLA and PLG of about 65:35.

In some embodiments, the biodegradable ocular implant of the disclosure comprises at least two PLGA. In some embodiments, the biodegradable polymer comprises at least three PLGA (e.g., three to six PLGA, three PLGA, four PLGA, five PLGA).

In some embodiments, the biodegradable ocular implant of the disclosure comprises at least two PLGA, wherein each PLGA is independently selected from the group consisting of RG502, RG502H, RG503, RG503H, RG504, RG504H, RG505, RG653H, RG750S, RG752H, RG752S, RG753H, RG753S, RG755S, RG756S, RG757S, and RG858S. In some embodiments, the biodegradable ocular implant of the disclosure comprises at least two PLGA in a ratio of about 99%: about 1% (e.g., about 98%: about 2%, about 97%: about 3%, about 96%: about 4%, about 95%: about 5%, about 94%: about 6%, about 95%: about 5%, about 94%: about 6%, about 93%: about 7%, about 92%: about 8%, about 91%: about 9%, about 90%: about 10%, about 90%: about 10%, about 89%: about 11%, about 88%: about 12%, about 87%: about 13%, about 87%: about 13%, about 86%: about 14%, about 85%: about 15%, about 84%: about 16%, about 83%: about 17%, about 82%: about 18%, about 81%: about 19%, about 80%: about 20%, about 79%: about 21%, about 78%: about 22%, about 77%: about 23%, about 76%: about 24%, about 75%: about 25%, about 74%: about 26%, about 73%: about 27%, about 72%: about 28%, about 71%: about 29%, about 70%: about 30%, about 69%: about 31%, about 68%: about 32%, about 67%: about 33%, about 66%: about 34%, about 65%: about 35%, about 64%: about 36%, about 63%: about 37%, about 62%: about 38%, about 61%: about 39%, about 60%: about 40%, about 59%: about 41%, about 58%: about 42%, about 57%: about 43%, about 56%: about 44%, about 55%: about 45%, about 54%: about 46%, about 53%: about 47%, about 52%: about 48%, about 51%: about 49%, about 50%: about 50%, about 49%: about 51%, about 48%: about 52%, about 47%: about 53%, about 46%: about 54%, about 45%: about 55%, about 44%: about 56%, about 43%: about 57%, about 42%: about 58%, about 41%: about 59%, about 40%: about 60%, about 39%: about 61%, about 38%: about 62%, about 37%: about 63%, about 36%: about 64%, about 35%: about 65%, about 34%: about 66%, about 33%: about 67%, about 32%: about 68%, about 31%: about 69%, about 30%: about 70%, about 29%: about 71%, about 28%: about 72%, about 27%: about 73%, about 26%: about 74%, about 25%: about 75%, about 24%: about 76%, about 23%: about 77%, about 22%: about 78%, about 21%: about 79%, about 20%: about 80%, about 19%: about 81%, about 18%: about 82%, about 17%: about 83%, about 16%: about 84%, about 15%: about 85%, about 14%: about 86%, about 13%: about 87%, about 12%: about 88%, about 11%: about 89%, about 10%, about 90%, about 9%: about 91%, about 8%: about 92%, about 7%: about 93%, about 6%: about 94%, about 5%: about 95%, about 4%: about 96%, about 3%: about 97%, about 2%: about 98%, and about 1%: about 99%). In some embodiments, the biodegradable ocular implant of the disclosure comprises at least two PLGA in a ratio of about 50% to about 75%: about 25% to about 50% (e.g., about 50% to about 70%: about 30% to about 50%, about 50% to about 65%: about 35% to about 50%, about 50% to about 60%: about 40% to about 50%, and about 55%: about 45%). In certain embodiments, the biodegradable ocular implant of the disclosure comprises at least two PLGA in a ratio of about 50%: about 50%. In embodiments, the two PLGA are RG503 and RG503H. In embodiments, the two PLGA are RG502 and RG502H. In embodiments, the two PLGA are RG504 and RG504H.

In some embodiments, the biodegradable polymer comprises at least three varying biodegradable polymers. In some embodiments, the biodegradable polymer comprises at least three PLGA, wherein each PLGA is independently selected from the group consisting of RG502, RG502H, RG503, RG503H, RG504, RG504H, RG505, RG653H, RG750S, RG752H, RG752S, RG753H, RG753S, RG755S, RG756S, RG757S, and RG858S. In some embodiments, the biodegradable polymer comprises at least three PLGA in a ratio of about 1% to about 95% (e.g., about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, and about 95%) : about 1% to about 95% (e.g., about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, and about 95%) : about 1% to about 95% (e.g., about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, and about 95%).

In some embodiments, the biodegradable polymer comprises at least three PLGA in a ratio of about 40%: about 40%: about 20%. In some embodiments, the biodegradable polymer comprises at least three PLGA in a ratio of about 50%: about 10%: about 40%. In some embodiments, the biodegradable polymer comprises at least three PLGA in a ratio of about 10%: about 50%: about 40%. In some embodiments, the biodegradable polymer comprises at least three PLGA in a ratio of about 40%: about 40%: about 20%. In some embodiments, the biodegradable polymer comprises at least three PLGA in a ratio of about 10%: about 50%: about 40%. In some embodiments, the biodegradable polymer comprises at least three PLGA in a ratio of about 20%: about 60%: about 20%. In some embodiments, the biodegradable polymer comprises at least three PLGA in a ratio of about 20%: about 50%: about 30%. In some embodiments, the biodegradable polymer comprises at least three PLGA in a ratio of about 15%: about 50%: about 35%. In some embodiments, the biodegradable polymer comprises at least three PLGA in a ratio of about 15%: about 45%: about 40%. In embodiments, each PLGA is independently selected from the group consisting of RG503, RG503H and RG753S. In embodiments, each PLGA is independently selected from the group consisting of RG502, RG503, and RG753S. In embodiments, each PLGA is independently selected from the group consisting of RG502, RG503, and RG752S. In certain embodiments, each PLGA is independently selected from the group consisting of RG502, RG503, and RG755S. In certain embodiments, each PLGA is independently selected from the group consisting of RG502, RG503, and RG756S.

In some embodiments, the biodegradable polymer comprises at least four varying biodegradable polymers. In some embodiments, the biodegradable polymer comprises at least four PLGA, wherein each PLGA is independently selected from the group consisting of RG502, RG502H, RG503, RG503H, RG504, RG504H, RG505, RG653H, RG750S, RG752H, RG752S, RG753H, RG753S, RG755S, RG756S, RG757S, and RG858S. In certain embodiments, the biodegradable polymer comprises at least four PLGA, wherein each PLGA is independently selected from the group consisting of RG502, RG503, RG753S, RG755S, RG756S, and RG858S. In certain embodiments, the biodegradable polymer comprises at least four PLGA, wherein each PLGA is independently selected from the group consisting of RG502, RG503, RG753S, and RG858S.

In some embodiments, the biodegradable polymer comprises at least four PLGA in a ratio of about 1% to about 95% (e.g., about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, and about 95%): about 1% to about 95% (e.g., about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, and about 95%): about 1% to about 95% (e.g., about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, and about 95%): about 1% to about 95% (e.g., about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, and about 95%). In some embodiments, the biodegradable polymer comprises at least four PLGA in a ratio of about 10% to about 30% (e.g., about 10%, about 15%, about 20%, about 25%, and about 30%): about 20% to about 40% (e.g., about 20%, about 25%, about 30%, about 35%, about 40%): about 20% to about 40% (e.g., about 20%, about 25%, about 30%, about 35%, about 40%): about 10% to about 30% (e.g., about 10%, about 15%, about 20%, about 25%, and about 30%). In some embodiments, the biodegradable polymer comprises at least four PLGA in a ratio of about 1% to about 20% (e.g., about 1%, about 5%, about 10%, about 15%, about 20%): about 40% to about 60% (e.g., about 40%, about 45%, about 50%, about 55%, about 60%): about 20% to about 40% (e.g., about 20%, about 25%, about 30%, about 35%, about 40%): about 1% to about 20% (e.g., about 1%, about 5%, about 10%, about 15%, about 20%).

In certain embodiments, the biodegradable polymer comprises at least four PLGA in a ratio of about 20%: about 30%: about 30%: about 20%. In certain embodiments, the biodegradable polymer comprises at least four PLGA in a ratio of about 10%: about 50%: about 30%: about 10%. Each of the four PLGA in the biodegradable polymer may independently selected from the group consisting of RG502, RG503, RG753S, RG755S, RG756S, and RG858S. In some embodiments, each PLGA is independently RG502, RG503, RG753S, or RG858S.

In some embodiments, the biodegradable polymer (e.g., PLGA) biodegrades substantially from about 1 month to about 24 months (e.g., about 2 months to about 24 months, about 5 months to 24 months, about 7 months to about 10 months, about 10 months to about 24 months, about 12 months to about 24 months, about 15 months to about 24 months, about 17 months to about 24 months, about 20 months to about 24 months, and about 22 months to about 24 months). In some embodiments, the biodegradable polymer (e.g., PLGA) biodegrades substantially from about 3 months to about 12 months (e.g., about 4 months to about 12 months, 5 months to about 12 months, about 5 months to about 12 months, about 6 months to about 12 months, about 7 months to about 12 months, about 8 months to about 12 months, about 9 months to about 12 months, about 10 months to about 12 months, and about 11 months to about 12 months). In some embodiments, the biodegradable polymer (e.g., PLGA) biodegrades substantially from about 12 months to about 18 months (e.g., about 13 months to about 18 months, about 14 months to about 18 months, about 15 months to about 18 months, about 16 months to about 18 months, and about 17 months to about 18 months). In some embodiments, the biodegradable polymer (e.g., PLGA) biodegrades substantially from about 3, 4, 5, 6, 7, 8, 9, 10, 11 or 12 months.

Also provided herein is a method for preventing, treating, or ameliorating an ocular neovascularization in a subject in need thereof comprises contacting a biodegradable ocular implant comprising a biodegradable polymer containing a compound incorporated therein; wherein the compound is a compound of Formula I, or a pharmaceutically acceptable salt thereof. In certain embodiments, the biodegradable polymer comprises at least three PLGA. In some embodiments, the biodegradable polymer comprises at least three PLGA in a ratio of about 50%: about 10%: about 40%. In some embodiments, the biodegradable polymer comprises at least three PLGA in a ratio of about 20%: about 20%: about 60%. In certain embodiments, the three PLGA are selected from the group consisting of RG503, RG502 and RG753S. In some embodiments, the concentration of the compound of Formula I in the biodegradable polymer is about 45% w/w, and the biodegradable polymer comprises RG503, RG502 and RG753S in a ratio of about 20%: about 20%: about 60%. In some embodiments, the concentration of the compound of Formula I in the biodegradable polymer is about 45% w/w, and the biodegradable polymer comprises RG503, RG502 and RG753S in a ratio of about 50%: about 10%: about 40%. In some embodiments, the compound of Formula I is an anhydrous crystalline form (e.g., Form 1, 3, or 4) or a monohydrate crystalline form (e.g., Form 2). In some embodiments, the compound of Formula I is an anhydrous crystalline form (e.g., Form 4).

Also provided herein is a method for preventing, treating, or ameliorating a vascular leakage in a subject in need thereof comprises contacting a biodegradable ocular implant comprising a biodegradable polymer containing a compound incorporated therein; wherein the compound is a compound of Formula I, or a pharmaceutically acceptable salt thereof. In certain embodiments, the biodegradable polymer comprises at least three PLGA. In some embodiments, the biodegradable polymer comprises at least three PLGA in a ratio of about 50%: about 10%: about 40%. In some embodiments, the biodegradable polymer comprises at least three PLGA in a ratio of about 20%: about 20%: about 60%. In certain embodiments, the three PLGA are selected from the group consisting of RG503, RG502 and RG753S. In some embodiments, the concentration of the compound of Formula I in the biodegradable polymer is about 45% w/w, and the biodegradable polymer comprises RG503, RG502 and RG753S in a ratio of about 20%: about 20%: about 60%. In some embodiments, the concentration of the compound of Formula I in the biodegradable polymer is about 45% w/w, and the biodegradable polymer comprises RG503, RG502 and RG753S in a ratio of about 50%: about 10%: about 40%. In some embodiments, the compound of Formula I is an anhydrous crystalline form (e.g., Form 1, 3, or 4) or a monohydrate crystalline form (e.g., Form 2). In some embodiments, the compound of Formula I is an anhydrous crystalline form (e.g., Form 4).

Also provided herein is a method for preventing, treating, or ameliorating a neovascular age-related macular degeneration in a subject in need thereof comprises contacting a biodegradable ocular implant comprising a biodegradable polymer containing a compound incorporated therein; wherein the compound is a compound of Formula I, or a pharmaceutically acceptable salt thereof. In certain embodiments, the biodegradable polymer comprises at least three PLGA. In some embodiments, the biodegradable polymer comprises at least three PLGA in a ratio of about 50%: about 10%: about 40%. In some embodiments, the biodegradable polymer comprises at least three PLGA in a ratio of about 20%: about 20%: about 60%. In certain embodiments, the three PLGA are selected from the group consisting of RG503, RG502 and RG753S. In some embodiments, the concentration of the compound of Formula I in the biodegradable polymer is about 45% w/w, and the biodegradable polymer comprises RG503, RG502 and RG753S in a ratio of about 20%: about 20%: about 60%. In some embodiments, the concentration of the compound of Formula I in the biodegradable polymer is about 45% w/w, and the biodegradable polymer comprises RG503, RG502 and RG753S in a ratio of about 50%: about 10%: about 40%. In some embodiments, the compound of Formula I is an anhydrous crystalline form (e.g., Form 1, 3, or 4) or a monohydrate crystalline form (e.g., Form 2). In some embodiments, the compound of Formula I is an anhydrous crystalline form (e.g., Form 4).

Also provided herein is a method for preventing, treating, or ameliorating a macular edema in a subject in need thereof comprises contacting a biodegradable ocular implant comprising a biodegradable polymer containing a compound incorporated therein; wherein the compound is a compound of Formula I, or a pharmaceutically acceptable salt thereof. In some embodiments, the biodegradable polymer comprises at least three PLGA. In certain embodiments, the three PLGA are selected from the group consisting of RG503, RG502 and RG753S. In some embodiments, the biodegradable polymer comprises at least three PLGA in a ratio of about 20%: about 20%: about 60%. In some embodiments, the biodegradable polymer comprises at least three PLGA in a ratio of about 50%: about 10%: about 40%. In some embodiments, the concentration of the compound of Formula I in the biodegradable polymer is about 45% w/w, and the biodegradable polymer comprises RG503, RG502 and RG753S in a ratio of about 20%: about 20%: about 60%. In some embodiments, the concentration of the compound of Formula I in the biodegradable polymer is about 45% w/w, and the biodegradable polymer comprises RG503, RG502 and RG753S in a ratio of about 50%: about 10%: about 40%. In some embodiments, the compound of Formula I is an anhydrous crystalline form (e.g., Form 1, 3, or 4) or a monohydrate crystalline form (e.g., Form 2). In some embodiments, the compound of Formula I is an anhydrous crystalline form (e.g., Form 4).

### Method of Making Implant

A method of making a biodegradable ocular implant described herein comprises subjecting a biodegradable polymer containing a compound via solvent casting, injection molding, or extrusion, wherein the compound is a compound of Formula I:

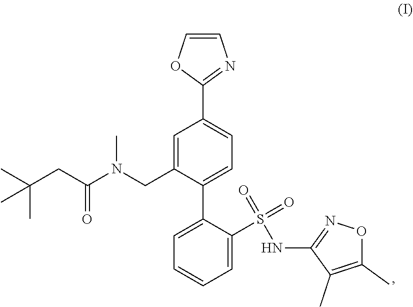 [SMILES: Cc1onc(NS(=O)(=O)c2ccccc2-c2ccc(-c3ncco3)cc2CN(C)C(=O)CC(C)(C)C)c1C]

or a pharmaceutically acceptable salt thereof.

Prior to implant fabrication blends of the polymer matrix and therapeutic agent may be dissolved and mixed with solvent to produce homogeneously dispersed therapeutic agent through the body of the implant. Prepared blends may each contain a different ratio of multiple, e.g., three, different PLGA polymers. The PLGA polymers used to produce the pharmaceutical compositions of the present invention may include, but are not limited to, RESOMER® RG502, RG503, RG752S RG753S, and 65/35 PLA/PLG, all of which are commercially available.

The following is an exemplary procedure used to prepare the compositions of the present invention: For example the polymers, in a particular ratio, are dissolved in an organic solvent, such as methylene chloride. The therapeutic agent (such as Edonentan) are then added to the polymer solution and dissolved. The methylene chloride is then evaporated in a polytetrafluoroethylene (PTFE) dish at room temperature. After the methylene chloride is evaporated, a thin film of homogeneous material remains. In an embodiment, the thin films range from 200 μm to 300 μm in thickness.

The remaining homogenous film is then milled to a powder using a cryogenic mill. Small portions of the film are added to stainless steel cryogenic milling vessels with 2 to 3 appropriately sized grinding balls and precooled using liquid nitrogen for 2 to 3 minutes at 5 Hz. The material is then milled for 1 minute from 20 Hz to 25 Hz with 1 minute of rest at 5 Hz. This milling/rest cycle is repeated from 2 to 5 times. The resulting material is a coarse to fine powder of homogenous material.

The implants of the present invention may be prepared, in an embodiment, using the homogenous material described above. In an embodiment, the implants are formed by injection molding. Injection molding can, for example, be performed by a suitable injection molder, such as a modified Haake MiniJet (ThermoFisher Scientific). The following is an exemplary procedure used to prepare the implants of the present invention.

The homogeneous powder is loaded and injected into a mold consisting of channels of an appropriate size, such as 300 μm×12 mm. The powder is loaded into a barrel leading into the mold and the mold placed under vacuum. The mold temperature is held from 15° C. to 75° C. The cylinder, surrounding the powder loaded barrel, is held from 145° C. to 220° C. for 10 to 15 minutes to melt the powder blend. The injection is performed using an injection pressure of 220 bar to 330 bar holding for 2 to 10 minutes. A post injection pressure is held at 50 bar from 2 to 10 minutes. The mold is then cooled down to 15 to 23° C. before removing the mold from the injection molder. The molded fibers are then removed from the mold and then cut into implants with a target weight and length. In some embodiments, the implants are 4 mm in length and contain about 165 μg to about 220 μg of active ingredient, such as Edonentan.

The implants of the present invention may be prepared, in an embodiment, using the homogenous material described above. In an embodiment, the implants are formed by extrusion for example, hot melt extrusion. Hot melt extrusion can be performed using ThermoFisher Pharma mini HME Micro Compounder, ThermoFisher FP-Pharma-11-Twin-230×100, ThermoFisher Pharma 11 Twin-Screw Extruder, ThermoFisher FP-Pharma-16-230×100, ThermoFisher Pharma 16 Twin-Screw Extruder, or Barrell Engineering Micro Syringe Type Extruder.

### Ocular Diseases

The methods of the present disclosure include the use of Edonentan and A-182086 described above in the prevention, treatment and amelioration of an ocular disease selected from the group consisting of an ocular neovascularization, vascular leak, neovascular age-related macular degeneration, neovascular glaucoma, retinal vein occlusion (RVO), and retinopathy of prematurity (ROP), which are described below.

As demonstrated herein, the therapeutic efficacy of the method is determined by the assessment of reduction in new vessel formation, or determined by reduction in the rate of ocular neovascularization. In further embodiments, the therapeutic efficacy of the method or treatment is indicated by an improvement in tissue, retinal perfusion, visual acuity, visual field, contrast sensitivity, or color vision.

### Neovascularization and Vascular Leakage

Ocular neovascularization, also called angiogenesis, occurs when abnormal blood vessels grow and spread throughout the retina and the tissue that lines the back of the eye and/or other structures in the eye (such as anterior chamber). These abnormal blood vessels are fragile and often leak, scarring the retina and pulling it out of position or cause blockade of aqueous humor drainage, resulting in increased intraocular pressure (i.e. neovascular glaucoma). An eye disorder in which neovascularization plays a role is age-related macular degeneration (AMD), which is the major cause of severe visual loss in the elderly. The vision loss in AMD results from choroidal neovascularization (CNV). The neovascularization originates from choroidal blood vessels and grows through Bruch's membrane, usually at multiple sites, into the sub-retinal pigmented epithelial space and/or the retina. Leakage and bleeding from these new blood vessels results in vision loss.

Ocular neovascularization, also called angiogenesis, occurs when abnormal blood vessels grow and spread throughout the retina, the tissue that lines the back of the eye and/or other structures in the eye (such as anterior chamber). These abnormal blood vessels are fragile and often leak, scarring the retina and pulling it out of position or cause blockade of aqueous humor drainage, resulting in increased intraocular pressure (i.e. neovascular glaucoma).

Types of the ocular neovascularization (e.g., choroidal neovascularization) include, but not limited to, neovascularization due to histoplasmosis and pathological myopia, angioid streaks, anterior ischemic optic neuropathy, bacterial endocarditis, Best's disease, birdshot retinochoroidopathy, choroidal hemangioma, choroidal nevi, choroidal nonperfusion, choroidal osteomas, choroidal rupture, choroideremia, chronic retinal detachment, coloboma of the retina, Drusen, endogenous Candida endophthalmitis, extrapapillary hamartomas of the retinal pigmented epithelium, fundus flavimaculatus, idiopathic, macular hole, malignant melanoma, membranoproliferative glomerulonephritis (type II), metallic intraocular foreign body, morning glory disc syndrome, multiple evanescent white-dot syndrome (MEWDS), neovascularization at ora serrata, operating microscope burn, optic nerve head pits, photocoagulation, punctate inner choroidopathy, rubella, sarcoidosis, serpiginous or geographic choroiditis, subretinal fluid drainage, tilted disc syndrome, Toxoplasma retinochoroiditis, tuberculosis, Vogt-Koyanagi-Harada syndrome, diabetic retinopathy, non-diabetic retinopathy, branch vein occlusion, central retinal vein occlusion, retinopathy in premature infants, rubeosis iridis, neovascular glaucoma, perifoveal telangiectasis, sickle cell retinopathy, Eale's disease, retinal vasculitis, Von Hippel Lindau disease, radiation retinopathy, retinal cryoinjury, retinitis pigmentosa, retinochoroidal coloboma, corneal neovascularization due to herpes simplex keratitis, corneal ulcers, keratoplasty, pterigyia, and trauma.

In embodiments, the disorder of the ocular neovascularization or the vascular leakage can be edema or neovascularization for any occlusive or inflammatory retinal vascular disease, such as rubeosis irides, neovascular glaucoma, pterygium, vascularized glaucoma filtering blebs, conjunctival papilloma; choroidal neovascularization, such as neovascular age-related macular degeneration (AMD), myopia, prior uveitis, trauma, or idiopathic; macular edema, such as post-surgical macular edema, macular edema secondary to uveitis including retinal and/or choroidal inflammation, macular edema secondary to diabetes, and macular edema secondary to retinovascular occlusive disease (i.e. branch and central retinal vein occlusion); retinal neovascularization due to diabetes, such as retinal vein occlusion, uveitis, ocular ischemic syndrome from carotid artery disease, ophthalmic or retinal artery occlusion, sickle cell retinopathy, other ischemic or occlusive neovascular retinopathies, retinopathy of prematurity, or Eale's Disease; and genetic disorders, such as VonHippel-Lindau syndrome.

In some embodiments, ocular neovascularization is associated with a condition selected from the group consisting of retinopathy of prematurity, retinal vein occlusion, macular edema, sickle cell retinopathy, choroidal neovascularization, radiation retinopathy, neovascular glaucoma, microangiopathy, retinal hypoxia, diabetic retinopathy, diabetic macular edema, ablation induced neovascularization, age related macular degeneration, and vascular leak.

In one embodiment, the neovascular age-related macular degeneration is a wet age-related macular degeneration. In another embodiment, the neovascular age-related macular degeneration is a dry age-related macular degeneration and the patient is characterized as being at increased risk of developing wet age-related macular degeneration.

In embodiments, the ocular neovascularization is associated with a condition selected from the group consisting of retinopathy of prematurity, retinal vein occlusion, macular edema, sickle cell retinopathy, choroidal neovascularization, radiation retinopathy, neovascular glaucoma, microangiopathy, retinal hypoxia, diabetic retinopathy, diabetic macular edema, ablation induced neovascularization, age related macular degeneration, and vascular leak.

### Neovascular Glaucoma

In the treatment of glaucoma (e.g., neovascular glaucoma) using Edonentan or A-182086 described herein, a “therapeutically effective amount” can be determined by assessing an improvement in retinal blood flow (RBF) over what could be achieved by the standard of care (lowering of intra-ocular pressure (TOP)). For a glaucoma (e.g., neovascular glaucoma) indication, the improvement in blood flow in the healthy rabbit ocular model can be used as predictive of pharmacodynamic response (PD) in humans. Rabbits are commonly used to assess ocular PK/PD relationship for compounds targeting human ocular diseases due to the anatomic and functional similarities of the rabbit and human eye. Previously, intravitreal administration of Endothelin 1 (ET-1) into the rabbit eye has been shown to induce significant vasoconstriction and optic nerve damage (Sasaoka M. et al, Exp Eye Res 2006; Sugiyama T. et al, Arch Ophthalmol 2009). Efficacy in this model is benchmarked to the reversal of perfusion impairment induced by intravitreal ET-1 administration at certain concentrations. For example, the efficacy can be achieved at a concentration equivalent to the levels observed in human glaucoma patients' plasma and aqueous humor (Li S. et al, Journal of Ophthalmology 2016).

Other examples of relevant animal glaucoma models are Morrison's rat model of elevated IOP and the laser-induced non-human primate (NHP) glaucoma model. Glaucoma in Morrison's rat model is induced by sustained elevation of IOP through hypertonic saline administration via episcleral veins. In the laser-induced NHP glaucoma model, after sustained elevation of IOP, optic nerve head blood flow has been shown to be reduced (Wang L. et al, Invest Ophthalmol Vis Sci 2012). Furthermore, the reduction in optic nerve head blood flow has been shown to correlate with long-term structural changes in the optic nerve (Cull G. et al, Invest Ophthalmol Vis Sci 2013).

Efficacy in the above-described glaucoma models is defined as reduction in IOP, improvement in optic nerve head or retinal blood flow from baseline, prevention or slowing of the progression of structural neurodegenerative changes such as retinal nerve fiber layer thickness as measured by optical coherence tomography (OCT) or retinal ganglion cell counts on flat mount as well as functional changes such as electroretinography (ERG) or contrast sensitivity after treatment with Edonentan or A-182086.

It is believed that the effect of Edonentan or A-182086 on retinal blood flow can be assessed by the blood vessel radius (r) in Poiseuille's Law. An increase in (r) with an endothelin antagonist, would induce a more pronounced increase in blood flow than what can be achieved by an increase in perfusion pressure through IOP reduction:
Blood flow=(perfusion pressure×πr⁴)/(8 ηl )
   where
l: blood vessel length
r: blood vessel radius
η: blood viscosity
perfusion pressure: mean arterial pressure—IOP
   Furthermore, Edonentan or A-182086 may reduce IOP and/or prevent RGC death through mechanisms independent of improvement in retinal/optic nerve head tissue perfusion. Accordingly, by using certain specific endothelin receptor antagonists, one (r) or more (TOP) of the above parameters can be altered to improve the RBF, thereby achieving therapeutic efficacy in treating glaucoma.

In some embodiments, the glaucoma patients are started on treatment as soon as they are diagnosed. In some embodiments, Edonentan or A-182086 is administered locally to the back of the eye using an intravitreal, topical, suprachoroidal, or implant delivery platform (e.g., a biodegradable ocular implant) with a frequency of every 3 to 12 (e.g., every 3 to 6 or every 4 to 6) months.

### Retinal Vein Occlusion (RVO)

Retinal vein occlusion (RVO), a vascular disorder of the retina, is currently treated with intravitreal injection of anti-VEGF drugs to inhibit the growth factor that causes macular edema and corticosteroids to combat the inflammatory components which lead to edema. It is highly desirable to use Edonentan and A-182086 therapies for treating RVO by improving tissue perfusion and reducing inflammation while avoiding the unwanted effects of systemic immunosuppression and/or local adverse effects of steroids.

RVO is currently treated with intravitreal steroids and anti-VEGF agents. We that improving perfusion of existing vessels will reduce the degree of macular edema and VEGF upregulation and the downstream maladaptive changes that manifests as RVO. To test efficacy, a preclinical mouse model of ischemic retinopathy can be used. Oxygen-induced retinopathy in the mouse is a reproducible and quantifiable proliferative retinal neovascularization model suitable for examining pathogenesis and therapeutic intervention for retinal neovascularization in many ischemic retinopathies including RVO. The model is induced by exposure of one-week-old C57BL/6J mice to 75% oxygen for 5 days and then to room air as previously described (Smith L E H et al., Invest Ophthalmol Vis Sci 1994). The efficacy in this preclinical model of ischemic retinopathy can be assessed by studying retinal hypoxia and neovascularization. A “therapeutically effective amount” of Edonentan or A-182086 described herein can be additive to the current standard of care by improving tissue perfusion and reducing inflammation mediated by ET-1 while avoiding the unwanted effects of local steroids. In some embodiments of treating RVO, the Edonentan or A-182086 is administered locally to the back of the eye using an intravitreal, topical, suprachoroidal, or implant delivery platform (e.g., a biodegradable ocular implant). The frequency of administration will vary based on a patient's disease course and response to therapy.

Retinopathy of prematurity (ROP)

Retinopathy of prematurity (ROP) is a retinal vasoproliferative disease that affects premature infants. ROP continues to be a major preventable cause of blindness and visual handicaps globally. With improved perinatal care, improved survival of moderately preterm infants, and limited resources for oxygen delivery and monitoring, more mature preterm infants are developing severe ROP in developing countries.

The pathophysiology of ROP is characterized by two phases. Phase I ROP is due to vaso-obliteration beginning immediately after birth secondary to a marked decrease in VEGF and insulin-like growth factor-1 (IGF-1). Phase II begins around 33 weeks' postmenstrual age (PMA). During this phase, VEGF levels increase, especially if there is retinal hypoxia with increasing retinal metabolism and demand for oxygen leading to abnormal vasoproliferation. For advanced stages of ROP, laser ablation of avascular retina, early treatment of ROP (ETROP) protocol, intravitreal injection of anti-VEGF antibodies (e.g. bevacizumab) and vitrectomy are used to protect central vision and prevent retinal detachment. Long-term complications such as refractory errors, recurrence of ROP and risk of retinal detachment require continued follow-up with an ophthalmologist through adolescence and beyond.

ROP is induced by severe ischemia due to underdevelopment of retinal vessels secondary to premature birth. Therefore, as an aspect of the invention, we believe that improving perfusion of existing vessels with Edonentan or A-182086 will reduce the degree of ischemia and VEGF upregulation and the downstream maladaptive changes that manifests as ROP. To test efficacy, a preclinical mouse model of ROP can be used. Oxygen-induced retinopathy in the mouse is a reproducible and quantifiable proliferative retinal neovascularization model suitable for examining pathogenesis and therapeutic intervention for retinal neovascularization in ROP. The model is induced by exposure of one-week-old C57BL/6J mice to 75% oxygen for 5 days and then to room air as previously described (Smith L E H et al., Invest Ophthalmol Vis Sci 1994). The efficacy in this preclinical model of ROP can be assessed by studying retinal hypoxia and neovascularization. A “therapeutically effective amount” of Edonentan or A-182086, as described herein will be additive to the current standard of care by improving tissue perfusion and reducing pathologic neovascularization induced by VEGF. In some embodiments, the medication is administered locally to the back of the eye using an intravitreal, topical, suprachoroidal, or implant delivery platform (e.g., a biodegradable ocular implant) with a frequency of every 4 to 6 weeks as needed based on patient's disease course and response to therapy. For example, the medication is administered locally to the back of the eye using an intravitreal injection with a frequency of every 5 weeks as needed based on patient's disease course and response to therapy.

### Pharmaceutical Compositions

Some embodiments described herein relates to a pharmaceutical composition, that can include a therapeutically effective amount of one of Edonentan and A-182086, described herein, or a pharmaceutically acceptable salt thereof, and a pharmaceutically acceptable carrier, diluent, excipient or combination thereof. Such antagonist or its pharmaceutically acceptable salt can be in a crystalline form or an amorphous form, each of which can be for pharmacologically acceptable use.

The term “pharmaceutical composition” refers to a mixture of one or both compounds disclosed herein with other chemical components, such as diluents or carriers. The pharmaceutical composition facilitates administration of the compound to an organism. Pharmaceutical compositions will generally be tailored to the specific intended route of administration.

Some pharmaceutical compositions involve preparing a pharmaceutically acceptable salt. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts. For a review on pharmaceutically acceptable salts, see Berge et al., 66 J. PHARM. SCI., 1-19 (1977).

The term “pharmaceutically acceptable” defines a carrier, diluent, excipient, salt or composition that is safe and effective for its intended use and possesses the desired biological and pharmacological activity.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, levigating, emulsifying, encapsulating or entrapping processes. See, e.g., Encapsulation Processes, in: Food Powders, 2005, 199-299. Additionally, the active ingredients are contained in an amount effective to achieve its intended purpose. Compounds used in the pharmaceutical combinations disclosed herein may be provided as pharmaceutically acceptable salts.

It is preferred to administer the compounds or pharmaceutical compositions of this invention in a local manner either as a topical ophthalmic formulation or via injection of the compounds or pharmaceutical compositions directly to the ocular tissue, often in a depot or sustained release formulation. The manner of local administration can be intravitreal, suprachoroidal, periocular, or subconjunctival injection of a formulation, or use of an implant technology or topical application. For example, the compound is administered in a liposomal preparation that slowly releases the compound sustaining the desired pharmacological effects. Alternatively, polyvinyl alcohol nanoparticles can be prepared by well-known methods to afford a sustained or extended release-formulation for topical or intra-ocular application.

Furthermore, one may administer the compound in a targeted drug delivery system. Examples of a targeted drug delivery system include, but are not limited to, a biodegradable ocular implant consisting of Edonentan homogenously dispersed through a PLGA polymer. In some embodiments, the biodegradable ocular implant is a sustained release biodegradable ocular implant.

In some embodiments, the pharmaceutical composition is an ophthalmic preparation comprising a therapeutically effective amount of one or more endothelin receptor antagonists described herein, or a pharmaceutically acceptable salt thereof. As used herein, an “ophthalmic preparation” refers to a specialized dosage form designed to be instilled onto the external surface of the eye (topical), administered inside (intraocular) or adjacent (periocular) to the eye or used in conjunction with an ophthalmic device. In some embodiments, the ophthalmic preparation is in the form of a solution, suspension, or an ointment. In other embodiments, the ophthalmic preparation is in the form of a gel, a gel-forming solution, an ocular insert, a micro/nanoparticle preparations for topical or preferably intravitreal injection, or an implant.

In some embodiments, the ophthalmic preparation comprises a preservative. Examples of suitable preservatives include, but are not limited to, cationic wetting agents (e.g, benzalkonium chloride), organic mercurials (e.g., phenylmercuric nitrate, phenylmercuric acetate), organic acids or their esters (e.g., sorbic acid, esters of p-hydroxybenzoic acid such as methyl hydroxybenzoate, propylhydroxybenzoate), and alcohol substitutes (e.g., chlorobutanol, phenylethanol). The preservative can be present in the ophthalmic preparation in an amount in the range of about 0.002% w/v to about 0.5% w/v (e.g., 0.01-0.25% w/v). The ophthalmic preparation can further comprise a preservative aid. Examples of suitable preservative aid include, but are not limited to, ethylenediaminetetraacetic acid (EDTA).

In some embodiments, the ophthalmic preparation comprises one or more additional excipients or agents to impart viscosity or lubrication, stabilize the active ingredients against decomposition, increase solubility of an active or inactive ingredient, adjust tonicity, or act as solvent. Examples of excipients or agents for imparting viscosity or lubrication include hypromellose, carbomer 974P, hydroxyethyl cellulose (HEC), polyvinyl alcohol, sodium hyaluronate, sodium carboxymethyl cellulose, Carbopol 940, hydroxypropylmethyl cellulose (HPMC), poloxamer, xyloglucan, alginic acid, sodium alginate, gellan gum, cellulose acetate phthalate, and xantham gum. Examples of excipients or agents as stabilizers include sodium bisulfite, sodium metabisulfite, sodium thiosulfate, and sodium sulfate/sulfuric acid, which can act as antioxidants. Examples of excipients or agents as solubilizers include, but are not limited to, providone, creatinine, castor oil, and cyclodextrin (e.g., γ-cyclodextrin). Examples of excipients or agents for adjusting tonicity include, but are not limited to, sodium chloride, potassium chloride, calcium chloride dehydrate, magnesium chloride hexahydrate, sugars (e.g., sucrose, maltose, dextrose, etc.), glycerin, propylene glycol, mannitol, ascorbic acid, and acetylcysteine.

In some embodiments, the ophthalmic preparation comprises one or more buffers to adjust pH. Examples of buffers for adjusting pH include, but are not limited to, sodium citrate, monobasic sodium phosphate, dibasic sodium phosphate, boric acid, hepatahydrate, sodium acetate trihydrate, sodium citrate dihydrate, histidine, and phosphate buffered saline (PBS). The resulting composition can have a pH value of 5.0-8.5 (e.g., 5.0-6.0, 5.2-5.8, 6.0-8.0, 6.6-7.8, 6.2-8.2, and 6.2-7.5)

In some embodiments, the ophthalmic preparation comprises one or more surfactants. Examples of surfactants include sorbitan ether esters of oleic acid (e.g., polysorbate or Tween 20 and 80) and tyloxapol.

The volume that can be injected to a human eye at one time is around 50-90 μL through the intravitreal route, up to 450 μL through a subretinal route, up to 200 μL via suprachoroidal routes, and about 40-50 μL via topical route (e.g. topical administration as an eye drop). The needle used in these routes is typically 27 to 30 G in size. The dose depends on the concentration that can be formulated to fit this volume, potency, target efficacy and pharmacokinetic profile for each indication. Generally, the injections to the eye will not be administered at a frequency greater than once per month per eye. For topical administrations (e.g. eye drop), in most instances, the frequency of administration to the eye does not exceed more than once or twice a day.

In some embodiments, the intravitreal formulation will comprise a dose of the compound (e.g., endothelin receptor antagonist, such as compound of Formula I) in the range of about 1 μg to about 1 mg (e.g., about 1 μg, about 5 μg, about 10 μg, about 25 μg, about 50 μg, about 75 μg, about 100 μg, about 125 μg, about 150 μg, about 175 μg, about 200 μg, about 250 μg, about 500 μg, about 700 μg, and about 1 mg). A first exemplary formulation comprises about 1 μg to about 1 mg of a compound (e.g., endothelin receptor antagonist, such as compound of Formula I) described above, about 10 mM histidine HCl, about 10% α,α-trehalose dihydrate, and about 0.01% polysorbate 20. A second exemplary formulation comprises about 1 μg to about 1 mg of a compound (e.g., endothelin receptor antagonist, such as compound of Formula I), about 10 mM sodium phosphate, about 40 mM sodium chloride, about 0.03% polysorbate 20, and about 5% sucrose.

In some embodiments, the intravitreal formulation will comprise a dose of the compound (e.g., endothelin receptor antagonist, such as compound of Formula I) in the range of about 1 μg to about 500 μg (e.g., about 10 μg to about 500 μg, about 20 μg to about 500 μg, about 30 μg to about 500 μg, about 40 μg to about 500 μg, about 50 μg to about 500 μg, about 60 μg to about 500 μg, about 70 μg to about 500 μg, about 80 μg to about 500 μg, about 90 μg to about 500 μg, about 100 μg to about 500 μg, about 100 μg to about 500 μg, about 125 μg to about 500 μg, about 150 μg to about 500 μg, about 175 μg to about 500 μg, about 200 μg to about 500 μg, about 225 μg to about 500 μg, about 250 μg to about 500 μg, about 275 μg to about 500 μg, about 300 μg to about 500 μg, about 325 μg to about 500 μg, about 350 μg to about 500 μg, about 375 μg to about 500 μg, about 400 μg to about 500 μg, about 425 μg to about 500 μg, about 450 μg to about 500 μg, and about 475 μg to about 500 μg).

In some embodiments, the intravitreal formulation will comprise a dose of the compound (e.g., endothelin receptor antagonist, such as compound of Formula I) in the range of about 10 μg to about 500 μg. In some embodiments, the intravitreal formulation will comprise a dose of the compound (e.g., endothelin receptor antagonist, such as compound of Formula I) in the range of about 10 μg to about 300 μg. In some embodiments, the intravitreal formulation will comprise a dose of the compound (e.g., endothelin receptor antagonist, such as compound of Formula I) in about 1 μg, about 5 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 110 μg, about 120 μg, about 130 μg, about 140 μg, about 150 μg, about 160 μg, about 170 μg, about 180 μg, about 190 μg, about 200 μg, about 210 μg, about 220 μg, about 230 μg, about 240 μg, about 250 μg, about 260 μg, about 270 μg, about 280 μg, about 290 μg, about 300 μg, about 310 μg, about 320 μg, about 330 μg, about 340 μg, about 350 μg, about 360 μg, about 370 μg, about 380 μg, about 390 μg, about 400 μg, about 410 μg, about 420 μg, about 430 μg, about 440 μg, about 450 μg, about 460 μg, about 470 μg, about 480 μg, about 490 μg, and about 500 μg. A first exemplary formulation comprises about 10 μg to about 500 μg (e.g., 300 μg) of a compound (e.g., endothelin receptor antagonist) described above, about 10 mM histidine HC1, about 10% α,α-trehalose dihydrate, and about 0.01% polysorbate 20. A second exemplary formulation comprises about 10 μg to about 500 μg (e.g., 300 μg) of a compound (e.g., endothelin receptor antagonist), about 10 mM sodium phosphate, about 40 mM sodium chloride, about 0.03% polysorbate 20, and about 5% sucrose.

In some embodiments, the intravitreal formulation will comprise a dose of the compound (e.g., endothelin receptor antagonist, such as compound of Formula I) in the range of about 150 μg to about 300 μg. In some embodiments, the intravitreal formulation will comprise a dose of the compound (e.g., endothelin receptor antagonist, such as compound of Formula I) in the range of about 165 μg to about 220 μg (e.g., about 165 μg, about 170 μg, about 175 μg, about 180 μg, about 185 μg, about 190 μg, about 195 μg, about 200 μg, about 205 μg, about 210 μg, about 215 μg, and about 220 μg).

In some embodiments, the intravitreal formulation will comprise a dose of the compound (e.g., endothelin receptor antagonist, such as compound of Formula I) in the range of about 300 μg to about 600 μg. In some embodiments, the intravitreal formulation will comprise a dose of the compound (e.g., endothelin receptor antagonist, such as compound of Formula I)) in the range of about 330 μg to about 500 μg (e.g., about 330 μg, about 335 μg, about 340 μg, about 345 μg, about 350 μg, about 355 μg, about 360 μg, about 365 μg, about 370 μg, about 375 μg, about 380 μg, about 385 μg, about 390 μg, about 395 μg, about 400 μg, about 405 μg, about 410 μg, about 415 μg, about 420 μg, about 425 μg, about 430 μg, about 435 μg, about 440 μg, about 445 μg, about 450 μg, about 455 μg, about 460 μg, about 465 μg, about 470 μg, about 475 μg, about 480 μg, about 485 μg, about 490 μg, about 495 μg, and about 500 μg).

In further embodiments, the intravitreal formulation will comprise a dose of the compound (e.g., endothelin receptor antagonist) in the range of about 500 μg to about 4 mg (e.g., about 500 μg, about 725 μg, about 1 mg, about 1.5 mg, about 2 mg, about 2.5 mg, about 3 mg, and about 3.5 mg). A first exemplary formulation comprises about 500 μg to about 1 mg of a compound (e.g., endothelin receptor antagonist) described above, about 0.014% potassium phosphate monobasic, 0.08% sodium phosphate dibasic, 0.7% sodium chloride, 0.02% polysorbate, and 0.5% sodium carboxymethyl cellulose. A second exemplary formulation comprises about 500 μg to about 1 mg of a compound (e.g., endothelin receptor antagonist) described above, about 0.04% sodium phosphate monobasic monohydrate, about 0.3% sodium phosphate dibasic heptahydrate, 0.63% sodium chloride, and about 1% to about 2.3% sodium hyaluronate.

Without further elaboration, it is believed that one skilled in the art can, based on the above description, utilize the present invention to its fullest extent. The following specific examples, i.e., Examples 1-15, are therefore to be construed as merely illustrative, and not limitative of the remainder of the disclosure in any way whatsoever.

### EXAMPLES

### Example 1: Compound Physicochemical and Biochemical Characterization

Provided in Table 1 below are physicochemical and biochemical data for Edonentan and A-182086 described above. As indicated in Table 1, at pH 2, A-182086 has a solubility superior to that of Edonentan. On the other hand, at pH 7, Edonentan has a solubility superior to that of A-182086.

TABLE 1
Compound physicochemical and biochemical characterization

| | Edonentan | | A-182086 | |
|---|---|---|---|---|
| Compound | (MW = 537) | | (MW = 578) | |
| Functional Potency | ET_{A} IC₅₀ = 1.54 nM | | ET_{A} IC₅₀ = 0.63 nM | |
| for ET_{A} and ET_{B} | ET_{B} IC₅₀ = 590 nM | | ET_{B} IC₅₀ = 3.48 nM | |
| Receptors | High potency | | High potency | |
| | High specificity | | Low specificity | |
| Solubility at pH 2 | <0.54 | μg/mL; | 92.5 | μg/mL |
| | <1 | μM | 159.8 | μM |
| Solubility at pH 7^{a} | 326 | μg/mL; | 172.7 | μg/mL; |
| | 607 | μM | 298.4 | μM |
| Solubility in Ethyl | >8900 | mg/mL; | >10620 | mg/mL; |
| Acetate | >16753 | mM | >18351 | mM |

| | | |
|---|---|---|
| | Good | Good |
| Stability in Solid State | Stable | about 87% remaining/ |
| (2 h@125° C.) | | unstable |
| LogD @pH 7.4 | 1.48 | 2.30 |
| Permeability | log Pₑ = −5.9 | log Pₑ = −5.1 |
| (PAMPA - log Pₑ) | PSA = 109.66 | PSA = 105.61 |
| (PSA^{b}) | Mid-High | Mid-High |
| | Permeability | Permeability |

^{a}The data are from the amorphous form.
^{b}Calculated property that considers surface charge distributions (mainly O and N). Compounds with a PSA around 90 or below would be predicted to cross the blood-brain barrier.

In the above table, the physicochemical data, e.g., solubility, were obtained following standard protocols known in the field (see, e.g., Reis et al., Mini Rev Med Chem., 2010, 10(11):1071-6; Avdeef et al., Expert Opin Drug Metab Toxicol., 2005, 1(2):325-42; Bharate et al., Comb Chem High Throughput Screen., 2016, 19(6):461-9; and Jain et al., J Pharm Biomed Anal., 2013, 86:11-35.); and the biochemical data, i.e., potency for ET_{A}/ET_{B}, were obtained following the protocols known in the field (see, e.g., Kirkby et al., Br J Pharmacol., 2008, 153(6):1105-19; and Maguire et al., Br J Pharmacol., 2014, 171(24):5555-72.).

### Example 2: Formulation of Edonentan for Intravitreal Use in Rabbit

An appropriate amount of Edonentan is dissolved in neat PEG400, followed by addition of a 15% CD (HP-(β-cyclodextrin) solution. The final concentration of PEG400 is measured to be 20%. Target concentrations are 5 mg/ml and 0.5 mg/ml based on the amount of Edonentan. The resulting solution is filtered using a 0.25 micron filter.

### Example 3: Effects of Edonentan and ET-1 in a Rabbit Model

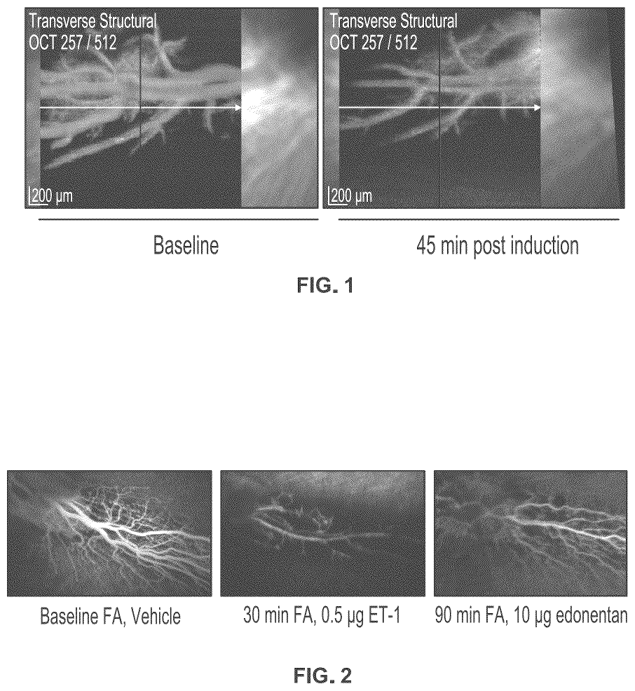

Adult, male Dutch-belted rabbits were given a 20 μl intravitreal injection (IVT) of 0.5 μg of ET-1 followed by a 20 μl intravitreal injection of 10-100 μg Edonentan given 30 min after the ET-1 administration. IOP, optical coherence tomography—angiography (OCT-A), and fluorescein angiograms (FA) were performed at pre-specified time points (30, 45, 60, and 75 min) following ET-1 and Edonentan administration to assess retinal blood flow changes induced by ET-1+/−Edonentan. As shown in FIG. 1, ET-1 administration effectively induced a clear vasoconstriction in the retinal vascular beds within 45 min. FIG. 2 shows that the effect of ET-1 was then reversed with 10 μg of Edonentan administration within 90 min (60 min after Edonentan administration).

### Example 4: Preparation of an Extended Release Formulation Containing Edonentan

A concentrated Edonentan dispersion is made by combining Edonentan with water, Vitamin E-TPGS and γ-cyclodextrin. These ingredients are mixed to disperse the Edonentan, and then autoclaved. Sodium hyaluronate may be purchased as a sterile powder or sterilized by filtering a dilute solution followed by lyophylization to yield a sterile powder. The sterile sodium hyaluronate is dissolved in water to make an aqueous concentrate. The concentrated Edonentan dispersion is mixed and added as a slurry to the sodium hyaluronate concentrate. Water is added in sufficient quaintly (q.s., as much as suffices, in this case as much as is required to prepare the homogenous mixture, dispersion, gel or suspension) and the mixture is mixed until homogenous. Examples of these compositions are provided in Table 2 below:

TABLE 2
Compositions of extended release
formulation containing Edonentan

| | Composition A | Composition B |
|---|---|---|
| Edonentan | 2.0% (w/v) | 8.0% (w/v) |
| Sodium hyaluronate (polymeric) | 2.5% (w/v) | 2.3% (w/v) |
| Sodium chloride | 0.63% (w/v) | 0.63% (w/v) |
| dibasic sodium phosphate, | 0.30% (w/v) | 0.30% (w/v) |
| heptahydrate | | |
| Monobasic sodium phosphate, | 0.04% (w/v) | 0.04% (w/v) |
| monohydrate | | |
| Water for injection | q.s. | q.s. |

These exemplary compositions contain a sufficient concentration of high molecular weight (i.e. polymeric) sodium hyaluronate so as to form a gelatinous plug or drug depot upon intravitreal injection into a human eye. Preferably the average molecular weight of the hyaluronate used is less than 2 million, and more preferably the average molecular weight of the hyaluronate used is between about 1.3 million and 1.6 million. The Edonentan particles are, in effect, trapped or held within this viscous plug of hyaluronate, so that undesirable pluming does not occur upon intravitreal injection of the formulation. Thus, the risk of drug particles disadvantageously settling directly on the retinal tissue is substantially reduced, for example, relative to using a composition with a water-like viscosity, such as Kenalog® 40. Since sodium hyaluronate solutions are subject to dramatic shear thinning, these formulations are easily injected via 25 gauge, 27 gauge or even 30 gauge needles.

### Example 5: Preparation of a Topical Edonentan Formulation

A topical Edonentan formulation can be prepared following a known method (e.g., WO 2016156639 A1). More specifically, 20 g of Cremophor® R/140 is dissolved in 75 mL of deionized water by magnetic stirring, which is allowed to stir until completely dissolved. Then 1.5 g of trometamol is added to the resulting solution and stirred for 15 minutes, achieving complete dissolution 0.5 g of Edonentan. is added and allowed to stir for 15 minutes, ensuring complete dissolution. Then 2 g of glycine and 1 g of boric acid are added and allowed to stir until completely dissolved. The resulting solution is added 100 mL deionized water in sufficient quantity. The final solution is filtered with filter paper, and a clear, colorless solution with a pH of 8.06 is obtained. The solution in dropper bottles eyedrop with a volume of 5 mL is packed.

### Example 6: Topical Ophthalmic Solution Nanoparticles Containing Edonentan

Nanoparticles were prepared by solvent evaporation technique. A solution of 120 mg of 50:50 PLGA in 60 mL of ethyl acetate was prepared. To this solution it was incorporated under turboagitation an aqueous solution of 50 ml of water with 12 mg of Edonentan and 0.5 mg of polyvinyl alcohol. The resulting mixture was left under continuous agitation and under vacuum for 2 hours. Then the resulting preparation was ultra-centrifuged and washed with water three times to remove the nanoparticles from the medium. The nanoparticles thus obtained were died in a vacuum oven and after evaluation, dispersed in an isotonic aqueous solution enough for a concentration of 5 mg/l mL of Edonentan.

### Example 7: Glaucoma Preclinical Studies

The healthy rabbit model is used to assess the pharmacodynamic effect (in vivo) of Edonentan and or A-182086 or pharmaceutically acceptable salts thereof. These studies are conducted with varying doses of the selected endothelin antagonists. Additional animal studies are conducted by combining endothelin antagonists with the current standard of care. The Morrison's rat model of glaucoma, rat model of acutely elevated IOP and laser induced glaucoma model in the non-human primate are used to assess optic nerve head blood flow and rate of retinal ganglion cell loss with varying doses of the selected endothelin antagonists with and without standard of care.

The improvement in blood flow in the healthy rabbit model is measured for the indicated endothelin receptor antagonists at varying doses after induction of perfusion impairment by locally administered ET-1. The changes in optic nerve head blood flow and retinal nerve fiber layer (RNFL) thickness in the non-human primate glaucoma models are measured for the indicated endothelin receptor antagonists at varying doses. The results show an improvement of RGC survival, retinal and optic nerve head blood flow and slowing of RNFL thinning due to the use of selected endothelin receptor antagonists. Dosing regimens for humans are predicted from the results of the healthy rabbit and non-human primate glaucoma models.

### Pharmacodynamic Study to Evaluate Changes Retinal Blood Flow in the Rabbit

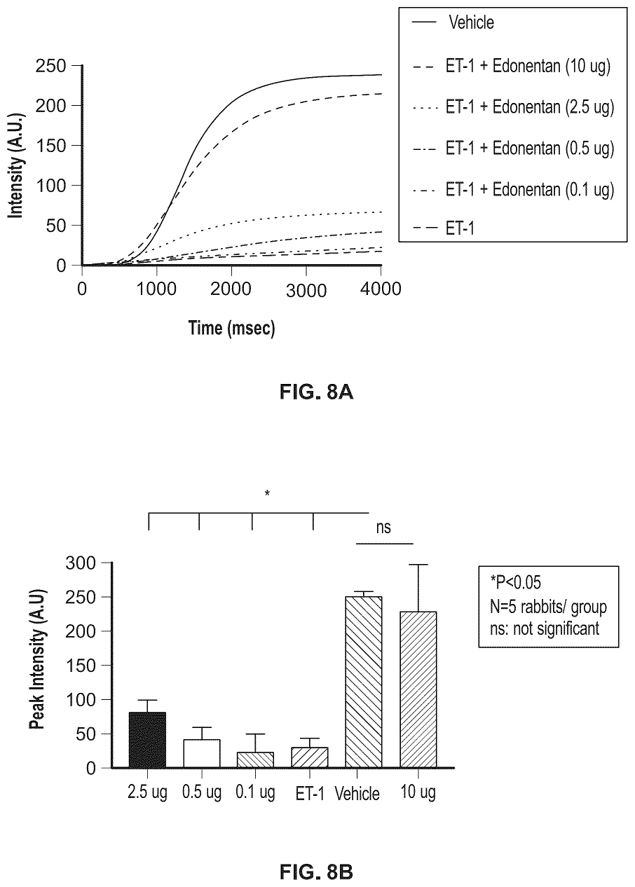

To evaluate the effect of retinal blood flow following intravitreally administered endothelin-1 (ET-1) followed by the antagonist Edonentan in the rabbit, rabbits (Oryctolagus cuniculus) were given a 20 μL intravitreal injection of ET-1 in the left eye followed by a 20 μL intravitreal injection of Edonentan at 2 (or 3) different doses (e.g. 0.1 μg, 0.5 μg, 2.5 μg). The pulse ox, tonometry, optical coherence tomography angiography (OCTA), fluorescein angiography (FA) and retinal leakage scoring were performed for evaluation. The dose-response in the rabbit is shown in FIG. 8A and FIG. 8B.

### Pharmacokinetic and Tolerability Analysis of Edonentan Delivered Intravitreally in the Rabbit

To determine the pharmacokinetic and safety properties of Edonentan following intravitreal administration in the rabbit, rabbits (Oryctolagus cuniculus) received bilateral intravitreal injections (20 μL injection volume/eye). Following the injections, animals were tranquilized with a ketamine/xylazine cocktail, and then the animals were euthanized with an overdose of sodium pentobarbital (Euthasol). Animals designated for the pharmacokinetic analysis were euthanized at different time points (e.g. 12, 16, 24, 36 and 48 hours). At least 1.0 mL of whole blood was drawn from the marginal ear vein or cardiac puncture (terminal bleed only) into K₂EDTA tubes for plasma collection and processed for analytical analysis.

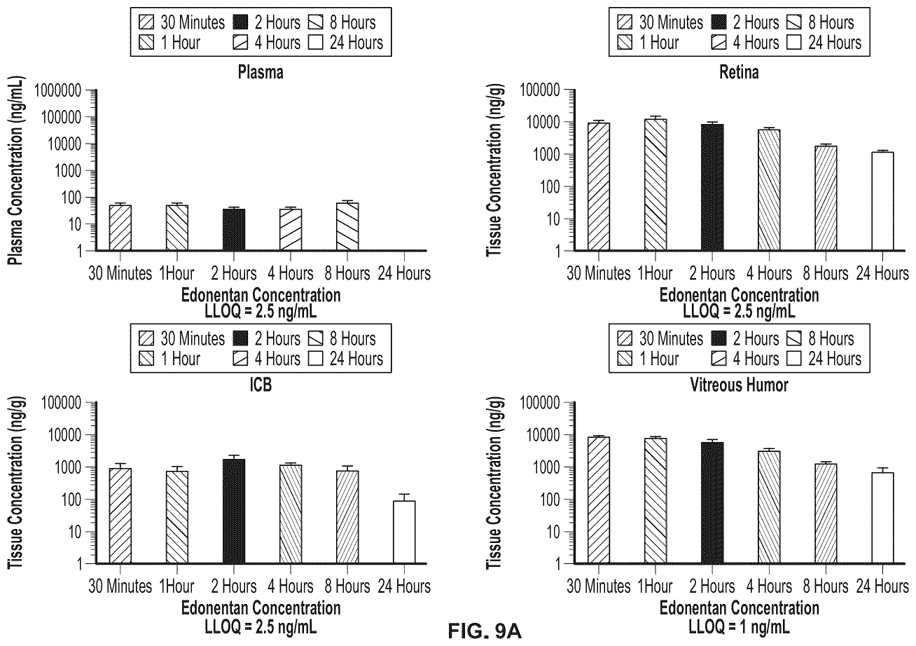

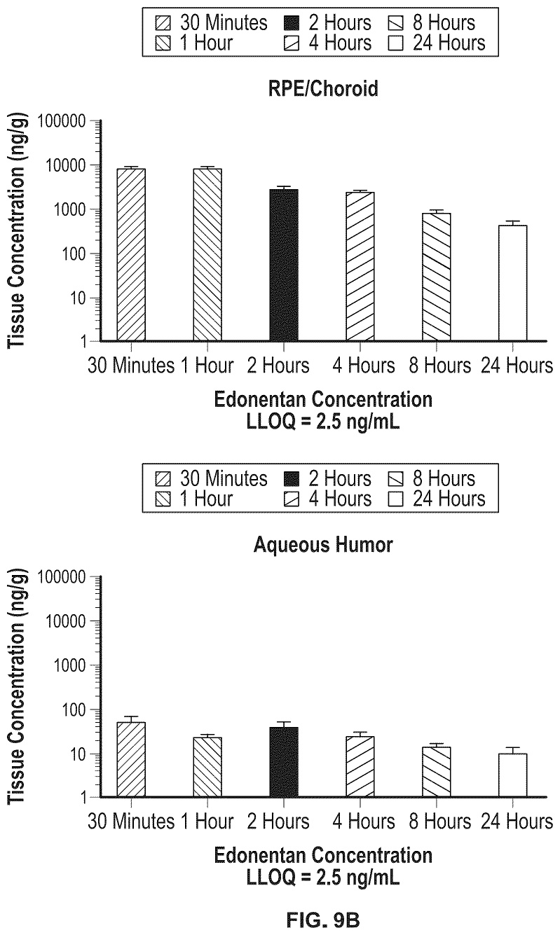

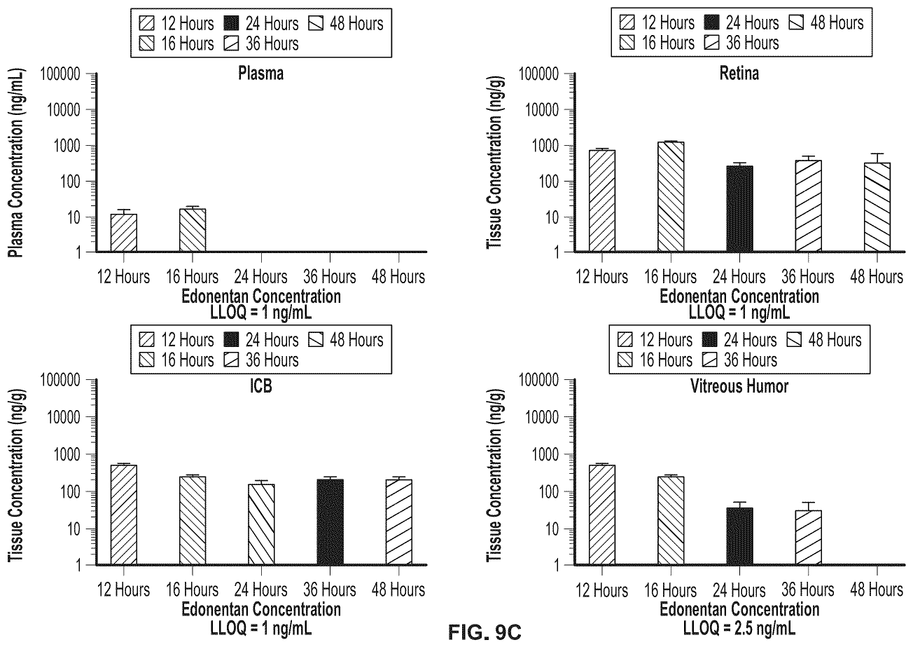

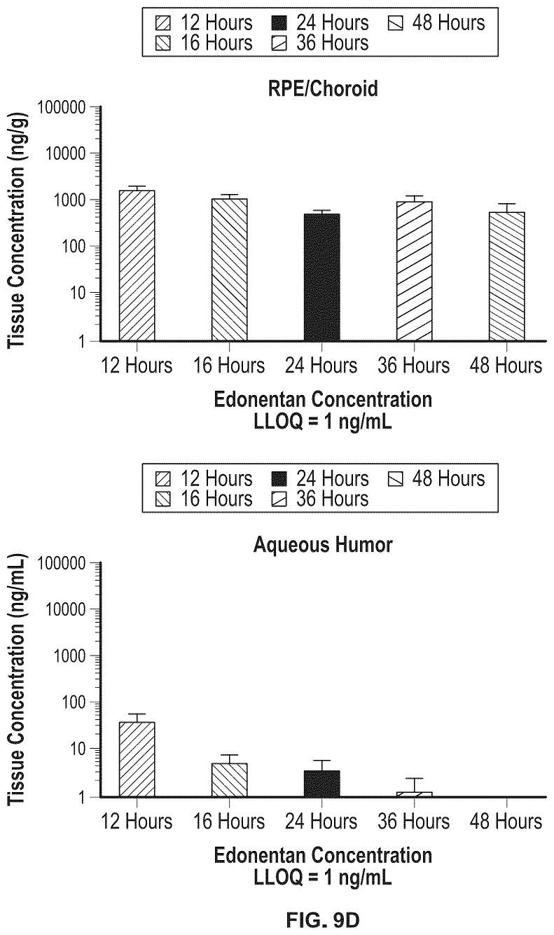

Immediately following euthanasia, the eyes was enucleated. Aqueous humor from both eyes was removed via syringe and snap frozen for analysis. Eyes were dissected when frozen to isolate various ocular tissues and minimize drug diffusion to adjacent tissues. Tissues from left and right eyes were collected in separate vials for analysis. List of tissues collected include plasma and aqueous humor, iris/ciliary body (ICB), retina, vitreous humor and RPE/choroid. The pharmacokinetic properties of intravitreally delivered Edonentan in rabbits are shown in FIG. 9A, FIG. 9B, FIG. 9C and FIG. 9D.

### Pharmacokinetic Analysis of Edonentan Administered Topically in the Rabbit

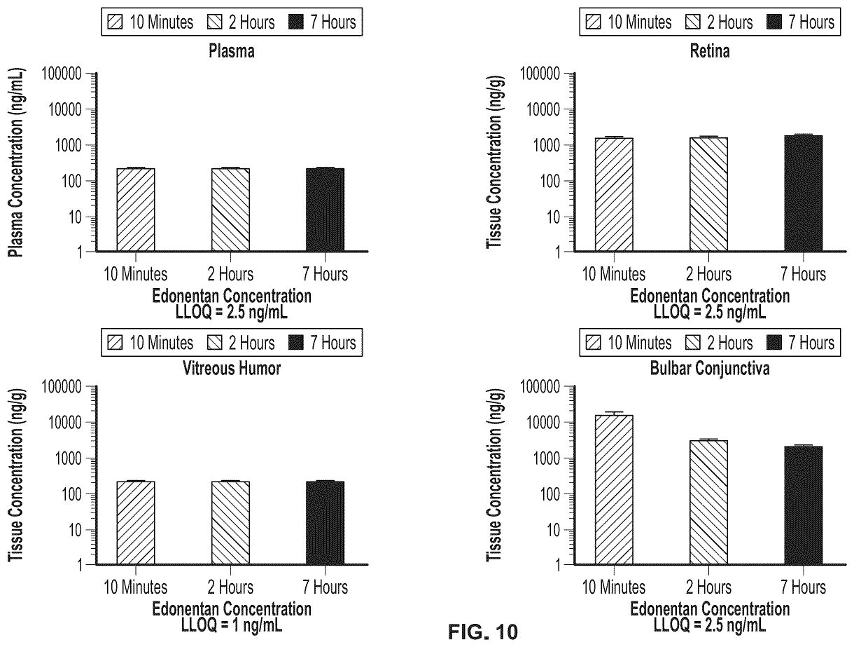

To determine the pharmacokinetic properties of Edonentan following topical administration in the rabbit, rabbits (Dutch-belted rabbits) received an eye drop in both eyes (100 μg of Edonentan, 35 μL dose volume/eye). After the administrations, the animals (N=2) were euthanized at different time points (e.g. 10 minutes (immediately after pot-dose), 2 and 7 hours) and tissues were collected for analysis. List of tissues collected include plasma, retina, vitreous humor and bulbar conjunctiva. The pharmacokinetic properties of topically delivered Edonentan in rabbits are shown in FIG. 10, which shows that Edonentan was detected in all tissues examined at all timepoints after a single topical application.

### Efficacy Study in the Morrison's Rat Model of Glaucoma

Adult male and female retired breeder Brown Norway rats (approximate age groups of 8 to 11 months) were obtained from Envigo (Indianapolis, IN). Baseline IOP measurements and pattern electroretinogram (PERG) amplitudes were collected prior to the surgery for elevation of IOP (to ensure that the IOP and PERG amplitudes were in the expected range of values). IOP was elevated in one eye (left eye) of the rats, while the corresponding right eyes served as contralateral controls. The Morrison method to elevate IOP in rats was carried out by injection of 50 μL of hypertonic saline through the episcleral veins to sclerose the trabecular meshwork. IOP was measured twice a week throughout the entire duration of the experiment. Seven to ten days following the surgery, IOP elevation was observed in the operated eye of rats. After detecting an elevation of IOP for two consecutive days, topical administration of eye drops (20 L (100 μg,) per dose of the tested compounds in the IOP elevated eye) was commenced and carried out for five days a week for a total of four weeks. In the 4th week of treatment, PERG analysis was carried out and rats were sacrificed by an overdose of pentobarbital (fatal-Plus). Aqueous humor was collected from the rat eyes, frozen and shipped for analysis. Retinal flat mounts were prepared, immunostained with the RGC marker, Brn3a antibody and surviving RG-Cs were counted in two eccentricities (central and peripheral).

For this study, the Morrison's model was used to induce ocular hypertension in adult male retired breeder Brown Norway rats as previously described by. Morrison et al., (Morrison J C, Moore C G, Deppmeier L M, Gold B G, Meshul C K, Johnson E C. A rat model of chronic pressure-induced optic nerve damage. Exp Eye Res. 1997;64(1):85-96).

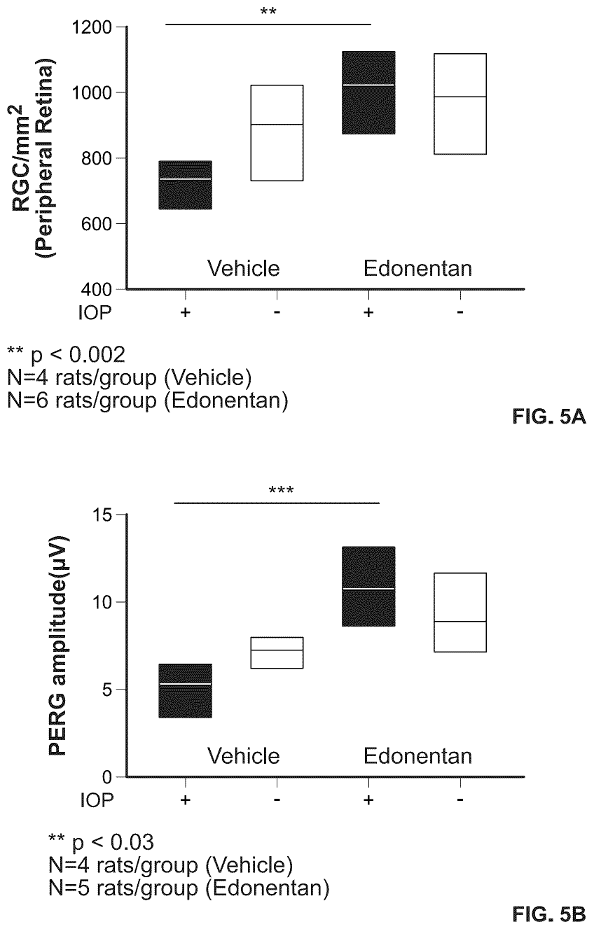

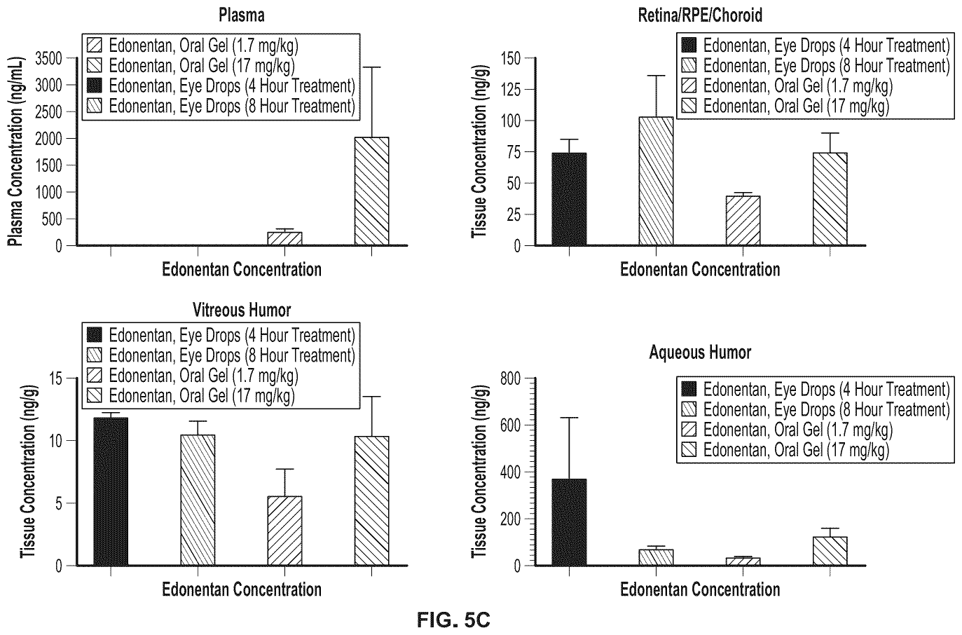

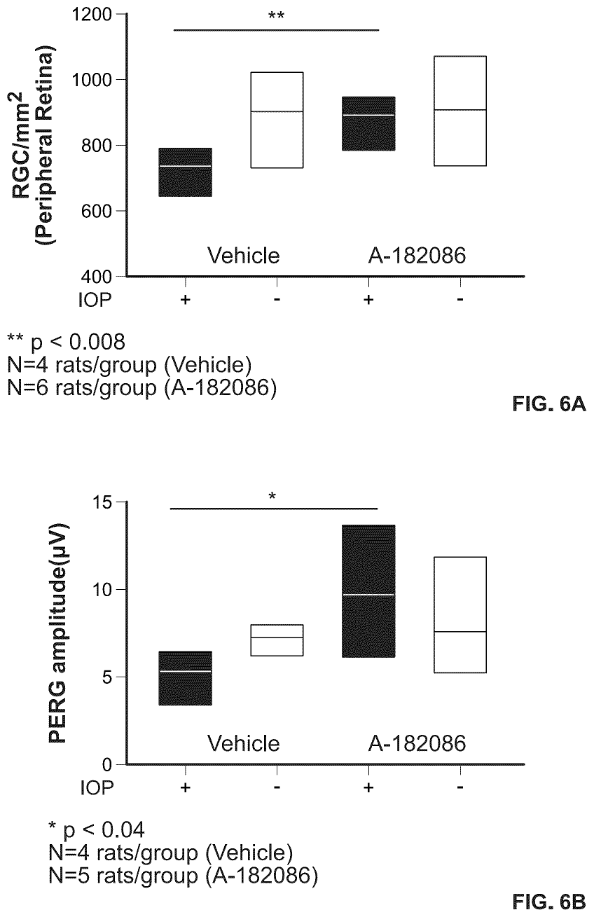

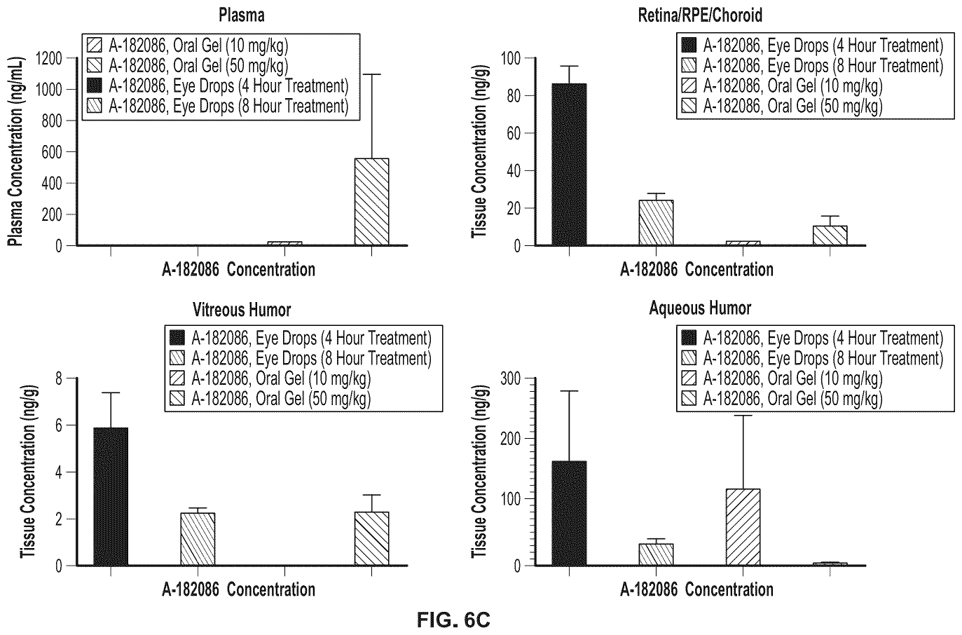

The immunostained retinal flat mounts were obtained to measure the retinal ganglion cell (RGC) counts. To obtain immunostained retinal flat mounts, the animals were euthanized after the treatments and then their eyes were enucleated. The eye cups were fixed overnight at 4° C. in 4% paraformaldehyde (PFA) and retinal flat mounts were prepared for collecting images. The retinal ganglion cell (RGC) counting was conducted using the images of immunostained retinal flat mounts. The images were uploaded to ImageJ, a photo editor designed for biology research (Rasband, 1997-2018) and the labeled retinal ganglion cells were counted manually in two eccentricities (central and peripheral). FIG. 5A shows the comparison of RGC counts in the peripheral retina between vehicle and Edonentan, and FIG. 6A shows the comparison between vehicle and A-182086.

Pattern ERG (PERG) was used to assess the RGC function. To obtain the pattern erg recordings, a UTAS Visual Electrodiagnostic System (LKC, Gaithersburgh, MD, USA) was used following the method described by Porciatti et al. (Porciatti V, Saleh M, Nagaraju M. The pattern electroretinogram as a tool to monitor progressive retinal ganglion cell dysfunction in the DBA/2J mouse model of glaucoma. Invest Ophthalmol Vis Sci. 2007;48(2):745-751). Briefly, PERG signals were acquired from a DTL-plus electrode placed on the lower part of the corneal surface and the PERG waves were analyzed using the EMWIN software (LKC). The difference between the amplitude of the major positive (P1) and negative (N2) waves were calculated to decipher the PERG amplitude. FIG. 5B shows IOP-mediated PERG changes between vehicle and Edonentan, and FIG. 6B shows the changes between vehicle and A-182086.

The RGC counts and PERG changes reveal that both Edonentan and A-182086 prevented RGC loss and maintained RGC function in the morrison's rat model of glaucoma, as shown in FIG. 5A, FIG. 5B, FIG. 6A and FIG. 6B.

### Pharmacokinetic Analysis of Edonentan or A-182086 Delivered Topically or Orally in the Rat

To determine the pharmacokinetic properties of Edonentan or A-182086 following topical administration in the rat, rats (Brown Norway rats) received an eye drop (100 μg of Edonentan, 20 μL dose volume/eye; or 100 μg of A-182086, 20 μL dose volume/eye). To determine the pharmacokinetic properties of Edonentan or A-182086 following oral administration in the rat, rats (Brown Norway rats) received an oral administration of 10 mg/kg or 50 mg/kg of Edonentan, or an oral administration of 1.7 mg/kg or 17 mg/kg of A-182086. After the administrations, the animals (N=2) were euthanized at different time points (e.g. 4 and 8 hours) and tissues were collected for analysis. List of tissues collected include plasma, retina/retinal pigment epithelium (RPE)/choroid, vitreous humor and aqueous humor. The pharmacokinetic properties of topically or orally administered Edonentan in rats is shown in FIG. 5C. The pharmacokinetic properties of topically or orally administered A-182086 in rats is shown in FIG. 6C. FIG. 5C and FIG. 6C show that both Edonentan and A-182086 are detected 4 and 8 hours post-topical administration in the retina/RPE/choroid, aqueous humor and vitreous humor. These data also revealed that Edonentan was detectable in the aqueous humor at the 17 mg/kg and in the retina/RPE/choroid and vitreous humor at 1.7 and 17 mg/kg, after oral administration of Edonentan, and A-182086 was detectable in the retina/RPE/choroid at 50 mg/kg, after oral administration of A-182086.

### Example 8: Laser-Induced Glaucoma, Non-human Primate Studies—Pharmacodynamic Study

Non-human primates (rhesus macaque, Macaca Mulatta) were obtained for this study. One eye of each animal underwent induction of elevation of intraocular pressure (TOP) by repeated laser photocoagulation of the trabecular meshwork. Imaging sessions were repeated to monitor the optic nerve head (ONH) and retinal structural changes.

Effect of Edonentan on optic nerve head blood flow after IVT administration

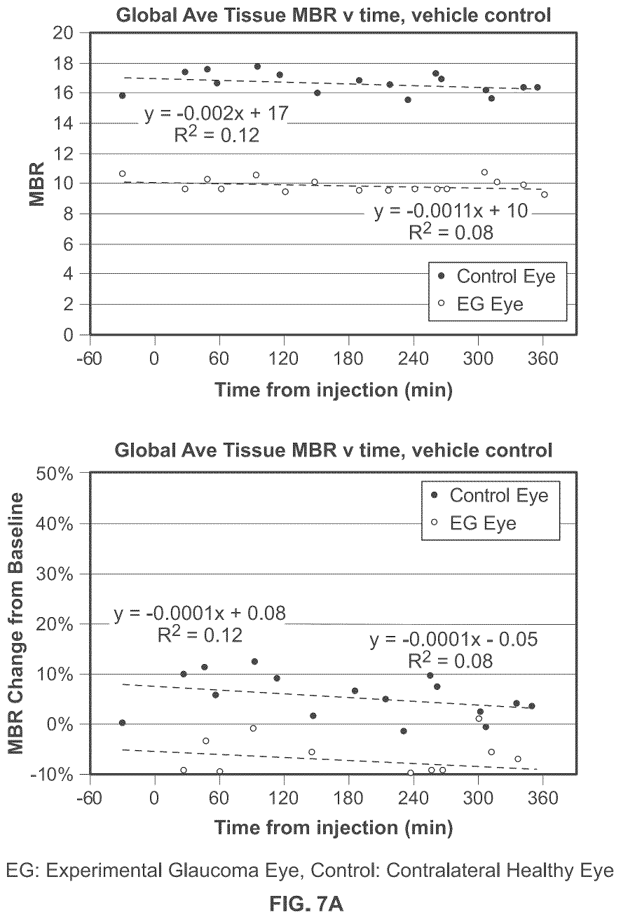

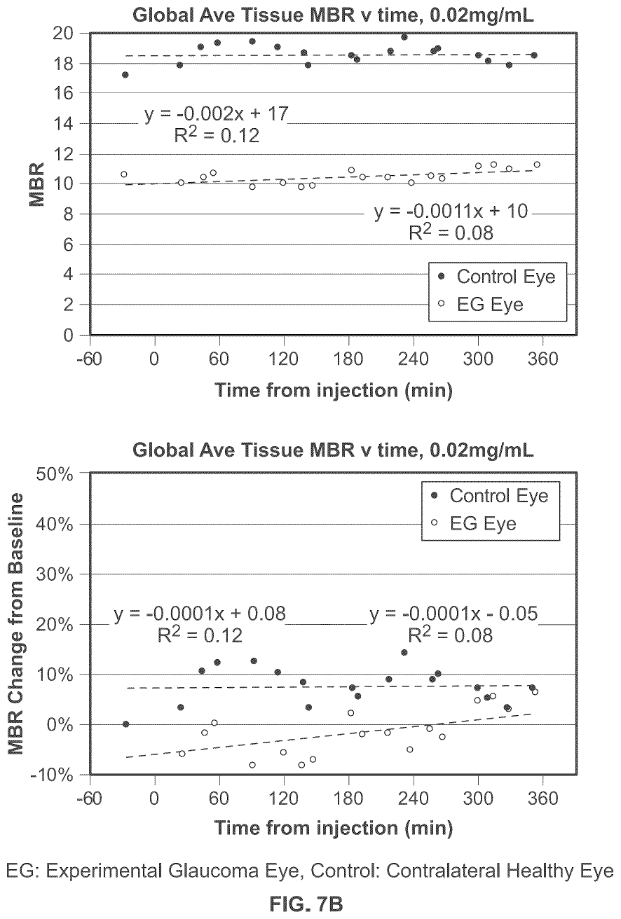

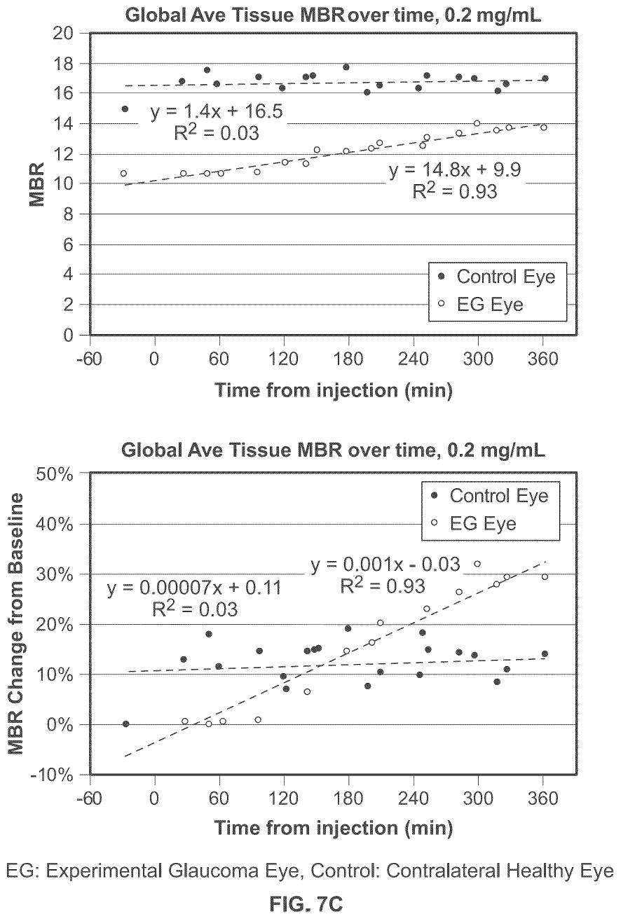

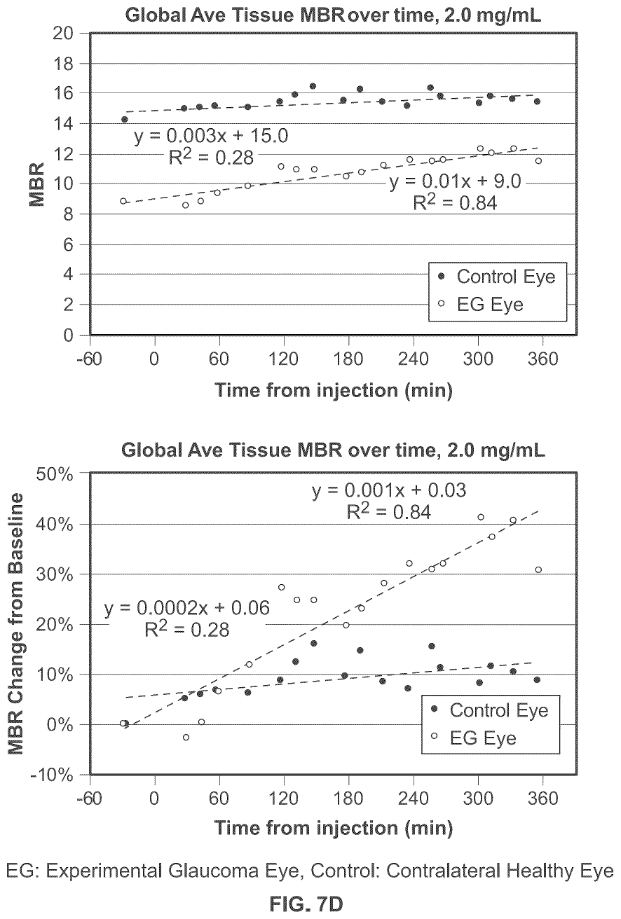

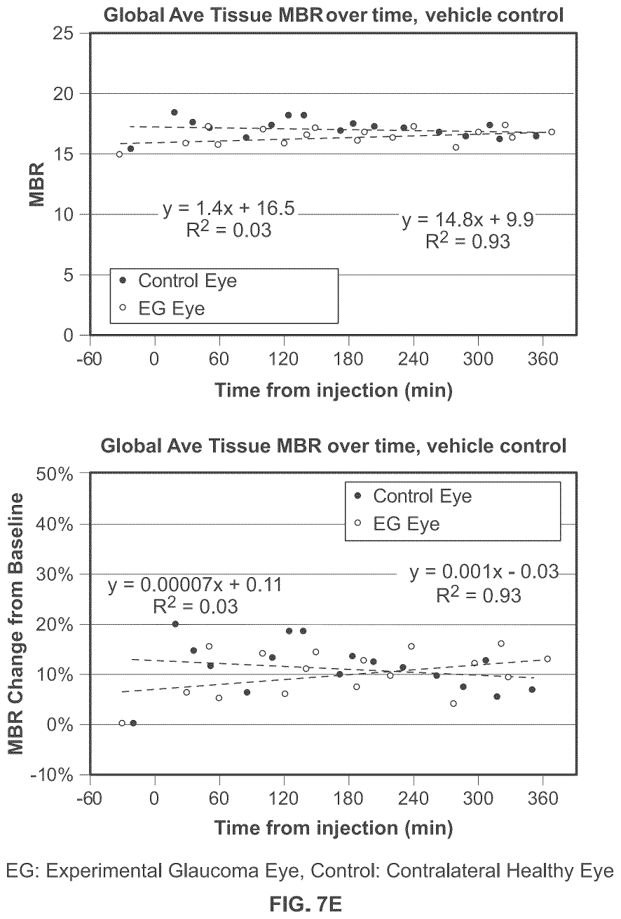

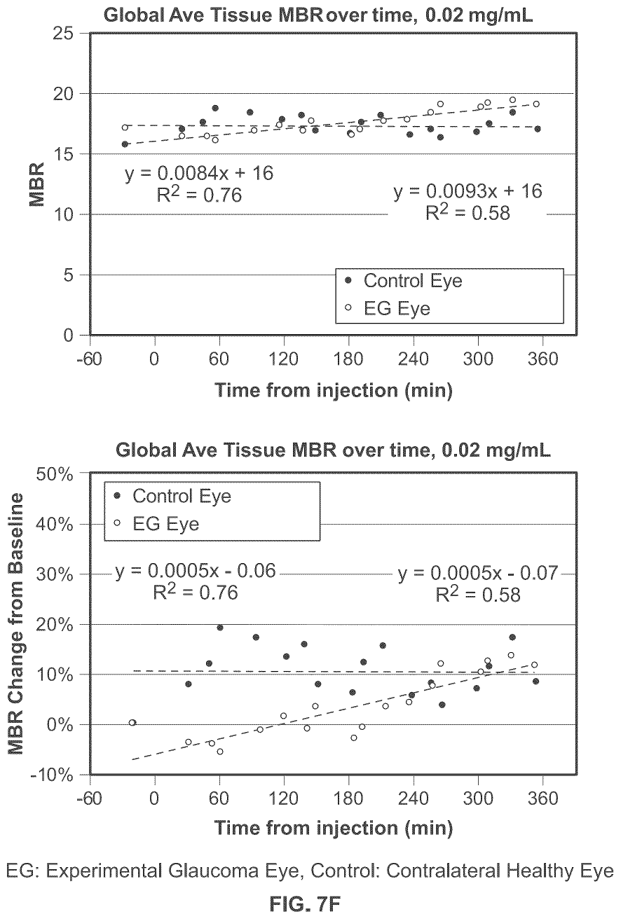

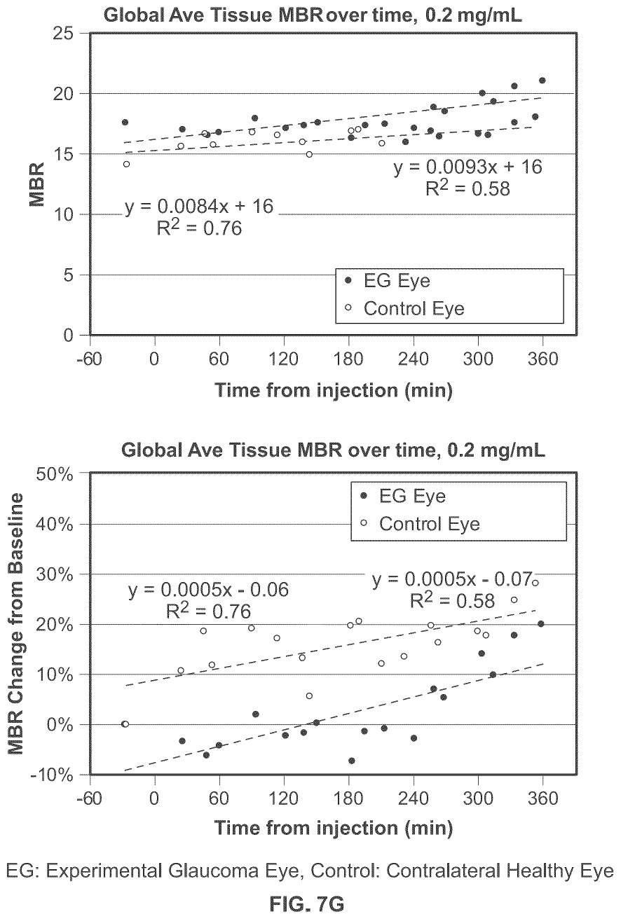

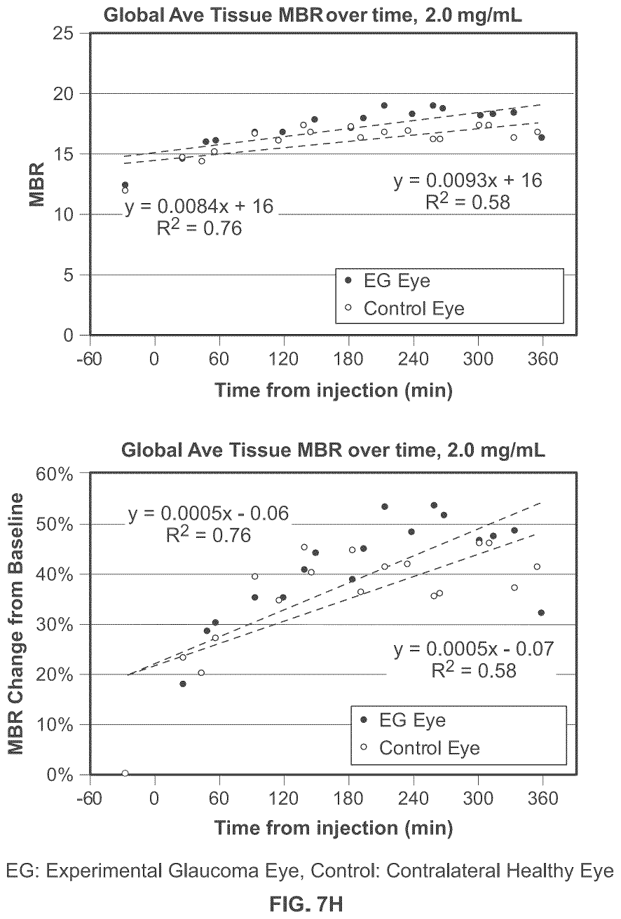

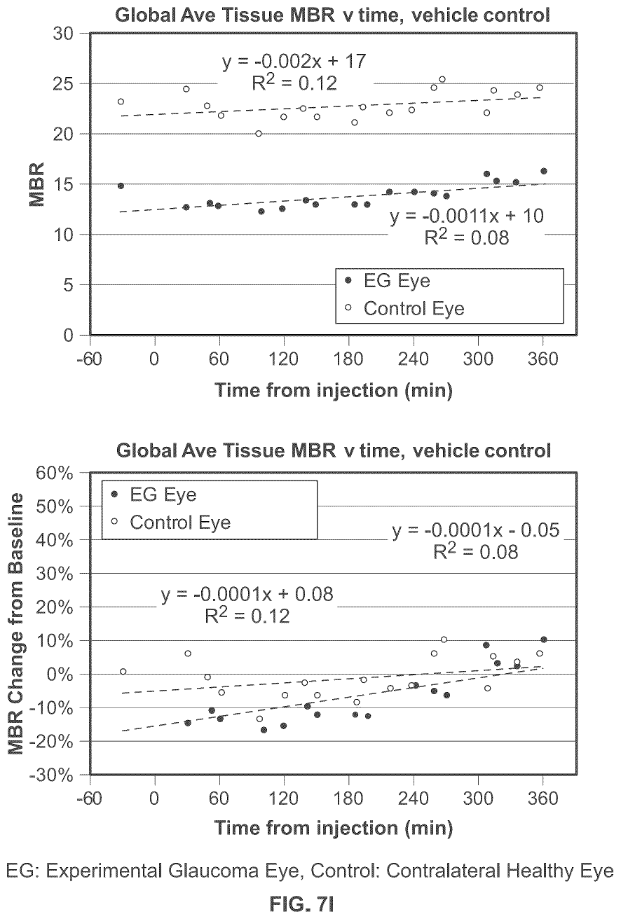

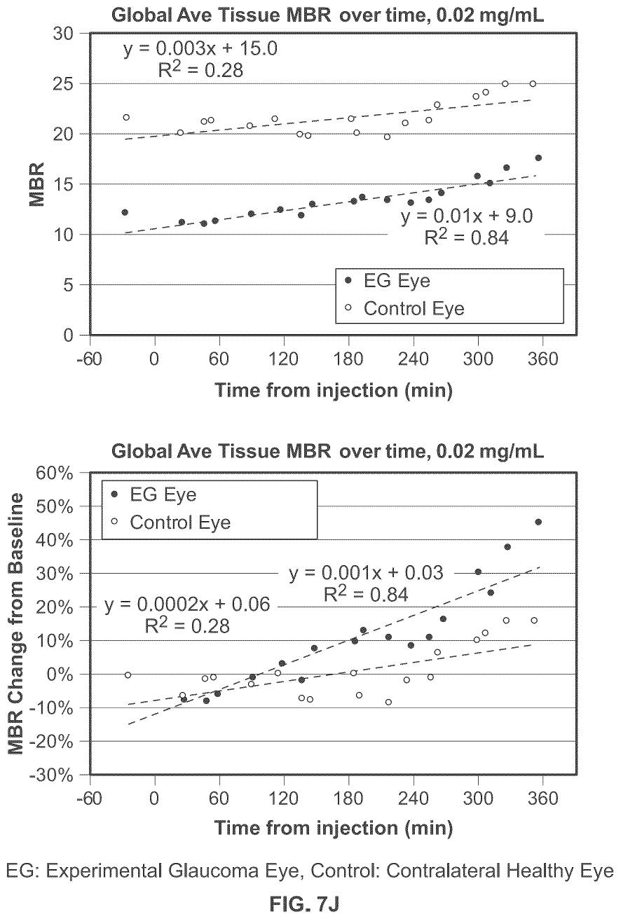

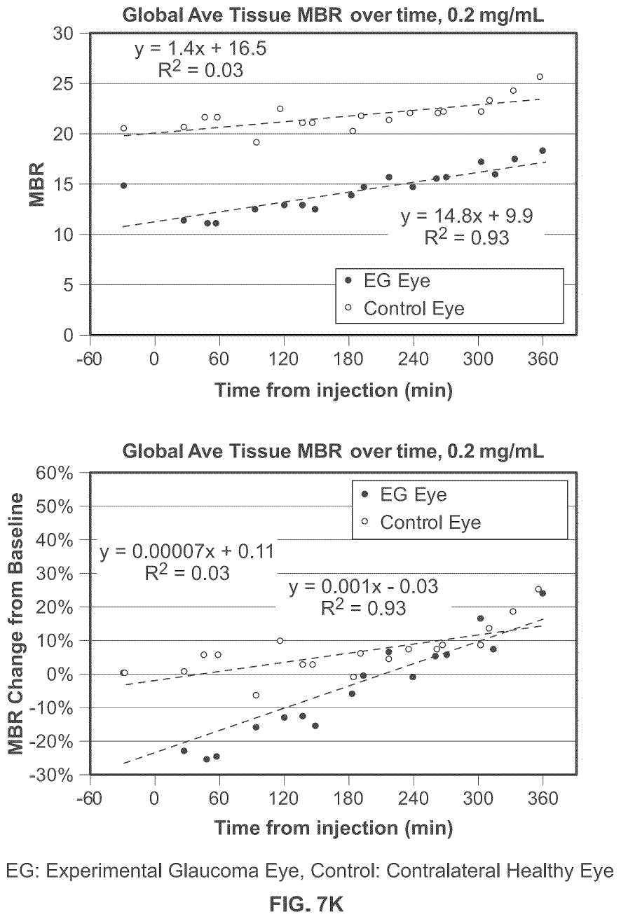

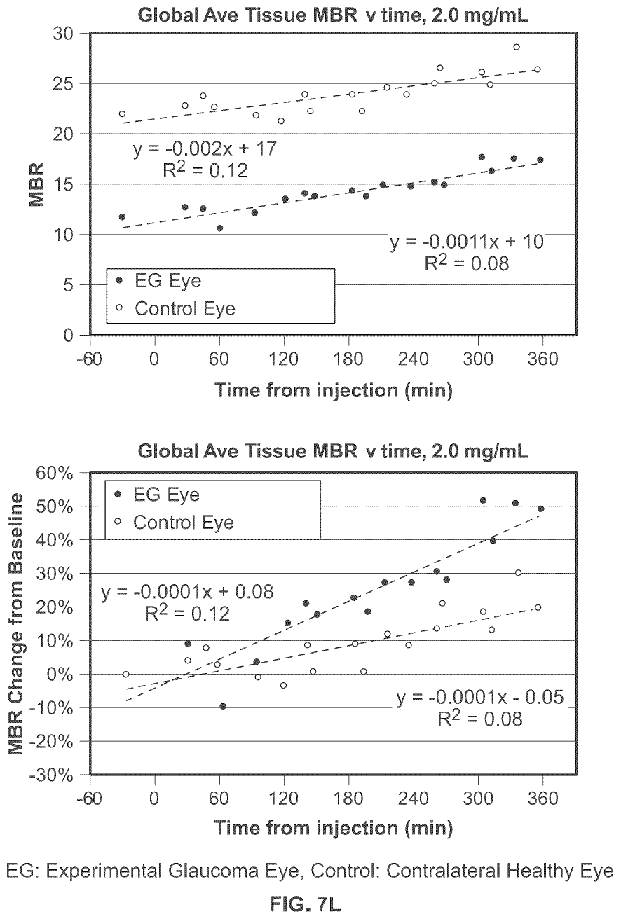

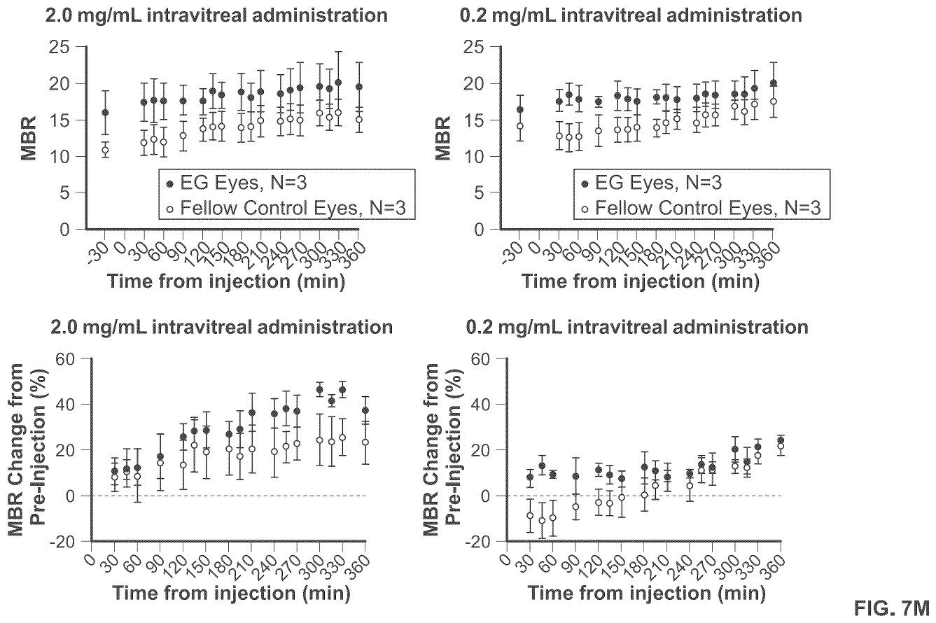

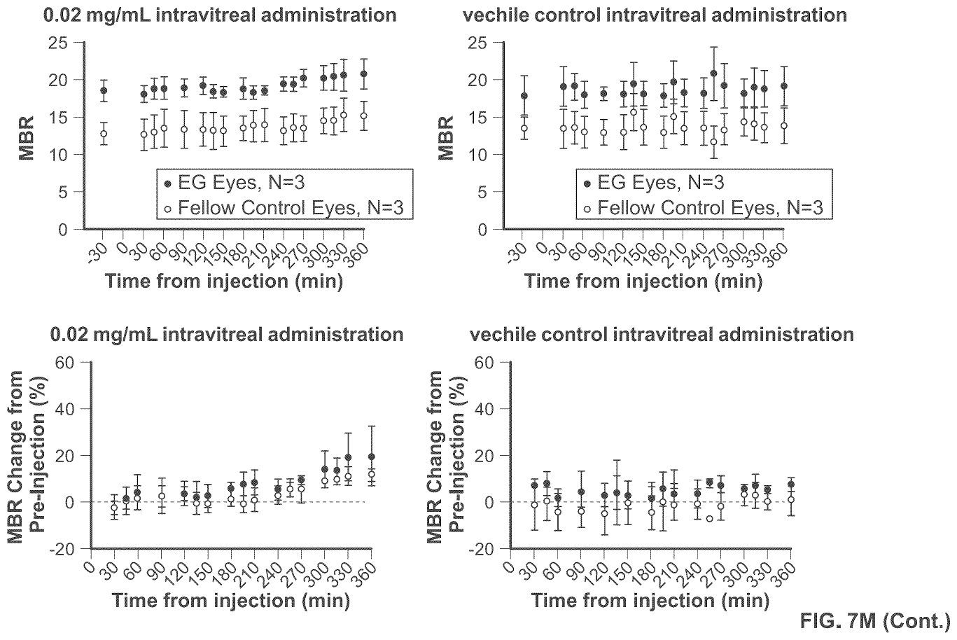

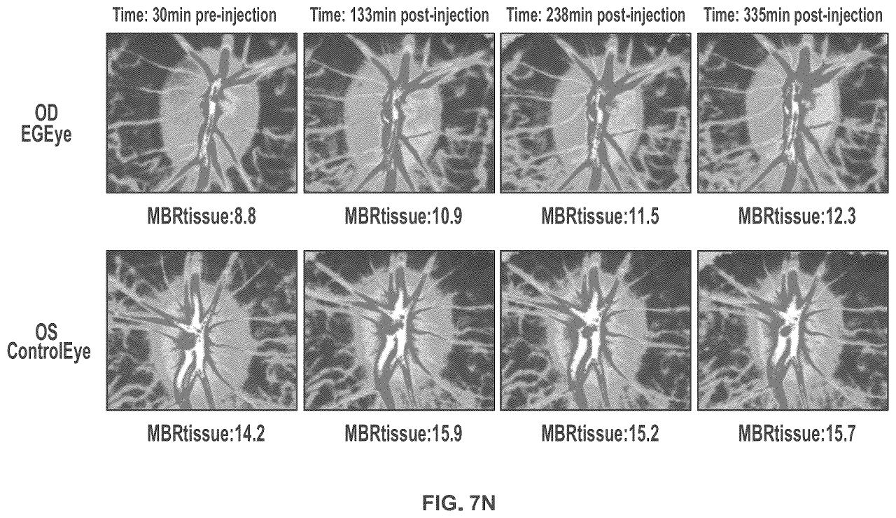

A study was performed to compare an experimental glaucoma eye and a contralateral healthy eye (control) of three non-human primates in global average mean blur rate (MBR) and MBR change from baseline over time as an index of ONH blood flow in a laser-induced glaucoma model. More specifically, a vehicle control, 0.02 mg/mL of Edonentan, 0.2 mg/mL of Edonentan, or 2.0 mg/mL of Edonentan was intravitreally administered (50 μL) to a glaucomatous eye of each of three non-human primates (rhesus macaque, Macaca Mulatta). The ONH blood flow was then measured over 6 hours using laser speckle flowgraphy (LSFG), as shown in FIGS. 7A-7L. These graphs show the ONH blood flow in the three non-human primates after IVT administration of a vehicle alone (FIG. 7A, FIG. 7E, and FIG. 7I), 0.02 mg/mL of Edonentan (FIG. 7B, FIG. 7F, and FIG. 7J), 0.2 mg/mL of Edonentan (FIG. 7C, FIG. 7G, and FIG. 7K) or 2.0 mg/mL of Edonentan (FIG. 7D, FIG. 7H, and FIG. 7L). FIGS. 7A-7L reveal the improvement of ONH blood flow in a dose-dependent manner after treatment with Edonentan. The aggregate results from the three non-human primates are shown in FIG. 7M, which show that Edonentan clearly exhibits does-related increase of ONH blood flow, resulting from dilation of retinal arteries, veins, and capillaries in experimental glaucoma eyes, as compared to control eyes.

In one of the above three non-human primates, an LSFG scan was performed at various selected time points when Edonentan was administered at 2.0 mg/mL. The results are shown in FIG. 7N.

Effect of Edonentan on Intraocular Pressure after Topical Administration

A single dose of 0.5% Timolol or a single dose of 2 mg/mL Edonentan was topically administered to three non-human primates that have laser-induced glaucoma in their right eyes (OD) with 1-week wash-out in a randomized order.

### Study Results:

Control 1: A single dose of 50 μL of topical Timolol 0.5% in each eye showed an IOP reduction of about 20% from pre-dose to post-dose (120 minutes).
Control 2: A single dose of 50 μL of topical Timolol 0.5% in each eye showed an IOP reduction of about 30% from pre-dose to post-dose (120 minutes).
Non-human Primate l: 50 μL of Edonentan eyedrop (2 mg/mL) in the experimental glaucoma eye showed an TOP reduction of about 60% from pre-dose to post-dose (120 minutes) and in the contralateral healthy eye showed an TOP reduction of about 10% from pre-dose to post-dose (120 minutes).
Non-human Primate 2: 50 μL of Edonentan eyedrop (2 mg/mL) in the experimental glaucoma eye showed an TOP reduction of about 50% from pre-dose to post-dose (15 minutes) and about 30% from pre-dose to post-dose (120 minutes). 50 μL of Edonentan eyedrop (2 mg/mL) in the contralateral healthy eye showed an TOP reduction of about 20% from pre-dose to post-dose (15 minutes) and about 0% from pre-dose to post-dose (120 minutes).
Non-human Primate 3: 50 μL of Edonentan eyedrop (2 mg/mL) in the experimental glaucoma eye showed an TOP reduction of about 40% from pre-dose to post-dose (15 minutes) and about 40% from pre-dose to post-dose (120 minutes). 50 μL of Edonentan eyedrop (2 mg/mL) in the contralateral healthy eye showed an TOP reduction of about 10% from pre-dose to post-dose (15 minutes) and about 40% from pre-dose to post-dose (120 minutes).

### Example 9: Formulation of Edonentan for Study in Mice with Oxygen-Induced Ischemic Retinopathy

A suitable topical formulation of Edonentan was prepared at concentrations of 0.05% w/w and 0.2% w/w active in a physiologically compatible system containing Hydroxypropyl Beta Cyclodextrin (HPβCD) and Sodium Carboxymethylcellulose (CMC), both available from Sigma-Aldrich. The HPβCD was dissolved in PBS, pH 7.4 at a concentration of 15% w/w. To this solution CMC, low molecular weight, was added at a concentration of 0.3% w/w. The solution was mixed until the polymers was fully dissolved and wetted. The active ingredient was then dissolved in an appropriate volume of 15% HPβCD with 0.3% w/w CMC. The active solution was placed in an autoclave and heated to 120° C. for 15 minutes and allowed to cool to room temperature. The solution was then filtered through a 0.22 μm PVDF filter.

### Example 10: Study in Mice with Oxygen-Induced Ischemic Retinopathy

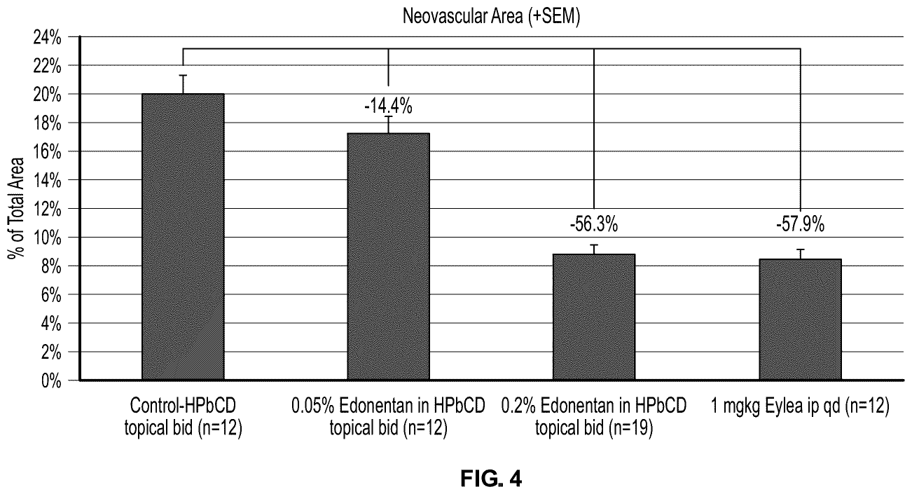

A mice model was used to obtain the retinal hypoxia area in mice with oxygen-induced ischemic retinopathy (OIR) at different time points, as shown in FIG. 4. 7-day old neonatal C57BL/6 mice were exposed to 75% oxygen from postnatal day (P)7 to P12. Upon return to normoxia on P12, mice were treated by twice daily topical eyedrops (5 μL) of edonentan (0.05% and 0.2% solution Example 9) or vehicle control, as well as once daily intraperitoneal injections with aflibercept at 1 mg/kg. Tissues were harvested following 5 days of treatment and stained for isolectin-IB4 for visualization and analysis of NV. A separate study was conducted to determine the drug levels achieved by 0.2% solution in retina and RPE/Choroid as the target therapeutic level.

### Example 11: Biodegradable Ocular Implant of Edonentan—Materials and Preparation Methods

Biodegradable implants were prepared using various grades of PLGA polymers. The polymers, in a particular ratio, were dissolved in methylene chloride. The therapeutic agent (such as edonentan) was then added to the polymer solution and dissolved. The methylene chloride was then evaporated in a polytetrafluoroethylene (PTFE) dish at room temperature. After the methylene chloride was removed a thin film of homogeneous material remained.

Exemplary polymers were in a particular ratio such as 50% RG503 and 50% RG503H (50/50 RG503/RG503H), was dissolved in methylene chloride. Edonentan, at 30% w/w, was then added to the polymer solution and dissolved. The methylene chloride was then evaporated in a polytetrafluoroethylene (PTFE) dish at room temperature for 72 to 120 hours. After the methylene chloride was removed, a thin film of homogeneous mixture of polymer and edonentan remained. The thin films could be from 200 μm up to 300 μm in thickness. The thin films were then cut into 3.5 mm long implants capable of being loaded into a 22 gauge needle. Implants were cut ranging in weight from approximately 200 μg up to 380 μis resulting in drug loads of 60 μis up to 114 μg.

### Example 12: Biodegradable Ocular Implant of Edonentan—Pharmacokinetic and Tolerability Analysis

Biodegradable ocular implant from Example 11 was designed for intravitreal delivery of edonentan over a period of 3 months. For in vitro drug release testing, three implants were incubated in 3 mL of PBS pH 7.4 in a shaking incubator set at 37° C. and 50 rpm. The drug release was sampled at designated time points and the drug content analyzed by an HPLC assay. The release medium was completely replaced with fresh medium during each sampling time point. Pharmacokinetics and tolerability of edonentan biodegradable implant were evaluated in rabbits for up to 21 days post-dose. Gross ophthalmic exams were conducted, and ocular matrices including remaining content in implants were processed and analyzed by LC-MS/MS at 14 and 21 days post-dose.

### Example 13: Biodegradable Ocular Implant of Edonentan—Materials and Preparation Methods

Using the procedure to produce homogeneous films in Example 11, additional formulations were prepared using injection molding and ram extrusion.

Exemplary polymers were in a particular ratio such as 50% RG503, 10% RG502 and 40% RG753S, was dissolved in methylene chloride. Exemplary formulations comprising various polymer and drug ratios are shown in Table 3. Edonentan, at 45% w/w, was then added to the polymer solution and dissolved. The methylene chloride was then evaporated in a polytetrafluoroethylene (PTFE) dish at room temperature for 24 hours and then dried under vacuum at 25° C. and 20 mbar for 24 hours. The films were then milled to a powder using a cryogenic mill. Small portions of the film were added to stainless steel cryogenic milling vessels with 2 to 3 appropriately sized grinding balls and precooled using liquid nitrogen for 2 or 3 minutes at 5 Hz. The material was then milled for 1 minute from 20 Hz to 25 Hz with 1 minute of rest at 5 Hz. This milling/rest cycle was repeated from 2 to 5 times. The resulting material was coarse to fine powder of homogenous material.

Implants were formed by injection molding with a modified Haake MiniJet (ThermoFisher Scientific). The homogeneous powder was loaded and injected into a mold consisting of channels of an appropriate size, such as 300 μm×12 mm or 325 μm×12 mm. The powder was loaded into a barrel leading into the mold and the mold placed under vacuum. The mold temperature was held at 15-25° C. The cylinder, surrounding the powder loaded barrel, was held from 145° C. to 165° C. for 12 to 15 minutes to melt the powder blend. The injection was performed using an injection pressure of 230 bar to 320 bar holding for 2 to 5 minutes. A post injection pressure was held at 50 bar from 2 to 5 minutes. The mold was then cooled to 15 to 23° C. before removing the mold from the injection molder. The molded fibers were then removed from the mold, and they were then cut into 4-mm implants containing 165 μg to 220 μg of Edonentan per implant.

Implants of select formulations were also formed by ram extrusion using a modified Barrell Micro Extruder (Barrell Engineering). The homogeneous powder was loaded into a 3 mm barrel and extruded through a 0.30 μm die maintaining a temperature of 68° C. to 80° C. and a flow rate of 5 μL/min to 6 μl/min. Extruded filaments were then cut into 4-mm implants containing 165 μg to 220 μg of Edonentan per implant. Resulting implants have similar performance characteristics as those produced with injection molding.

TABLE 3
Exemplary formulations.
Edonentan Containing Sustained Delivery Formulations (8-16) for the production of implants

| Formulation | Edonentan | Polymer % w/w | | | | | | | | |
|---|---|---|---|---|---|---|---|---|---|---|
| No. | % w/w | RG502 | RG503H | RG503 | RG752S | RG753S | RG755S | RG756S | RG858S | R203S |
| 8 | 30 | | 10 | 50 | | 40 | | | | |
| 9 | 45 | 10 | | 50 | | 40 | | | | |
| 10 | 45 | 20 | | 40 | | 40 | | | | |
| 11 | 45 | 10 | | 50 | 40 | | | | | |
| 12 | 45 | 20 | | 60 | | 20 | | | | |
| 13 | 45 | 20 | 20 | 40 | | 20 | | | | |
| 14 | 45 | | 10 | 50 | | 40 | | | | |
| 15 | 45 | 10 | 10 | 30 | | 50 | | | | |
| 16 | 45 | 20 | 20 | 20 | | 40 | | | | |
| 17 | 45 | 20 | | 20 | | 60 | | | | |
| 18 | 45 | 10 | | 50 | | | 40 | | | |
| 19 | 45 | 10 | | 50 | | | | 40 | | |
| 20 | 45 | 20 | | 30 | | 30 | | | 20 | |
| 21 | 45 | 10 | | 50 | | 30 | | | 10 | |
| 22 | 45 | 20 | | 30 | | 30 | | | | 20 |

### Example 14: Biodegradable Ocular Implant of Edonentan—Pharmacokinetic and Tolerability Analysis

Biodegradable ocular implants from Example 13 were designed for intravitreal delivery of edonentan over a period of 3 months. For in vitro drug release testing, three implants were incubated in 3 mL of PBS pH 7.4 in a shaking incubator set at 37° C. and 50 rpm. The drug release was sampled at designated time points and the drug content analyzed by an HPLC assay. The release medium was completely replaced with fresh medium during each sampling time point.

In a nonGLP 12-week ocular and systemic pharmacokinetic study in DB rabbits, 2 Edonentan Intravitreal Implants from either the injection molding (IM) or ram extrusion (RE) manufacturing process (total implant weight IM 423 μg/implant; 380 μg Edonentan/2 implants, RE 461 μg/implant, 415 μg Edonentan/2 implants) were administered as a single bilateral IVT injection in DB rabbits (2 animals and 4 eyes per timepoint). The implants contained 45% Edonentan in a blend of Resomer® containing 50% RG503, 10% RG502, and 40% RG753S. Rabbits were euthanized at Weeks 4, 8, 10, 11 and 12 and drug concentrations in aqueous humor, lens, vitreous humor, retina, RPE/choroid and plasma were determined.

Ocular tissue and plasma were analyzed for Edonentan content using an analytical method based on protein precipitation and liquid-liquid extraction followed by reverse-phase LC-MS/MS analysis. An Agilent 1290 UPLC coupled to an Agilent 6430 triple quadrupole mass spectrometer was used for analysis. The quantitation range for Edonentan was 1 to 250 μg/mL. Tissue and plasma samples were homogenized and extracted with 0.1% formic acid in acetonitrile which was spiked with deuterated Edonentan at approximately 10 ng/mL. The extracts were analyzed using reversed-phase liquid chromatographic separation with tandem mass spectrometric detection in the positive ion mode following the quantitative transition m/z 537.2 to 439.1 for Edonentan and m/z 540.2 to 442.1 for deuterated Edonentan.

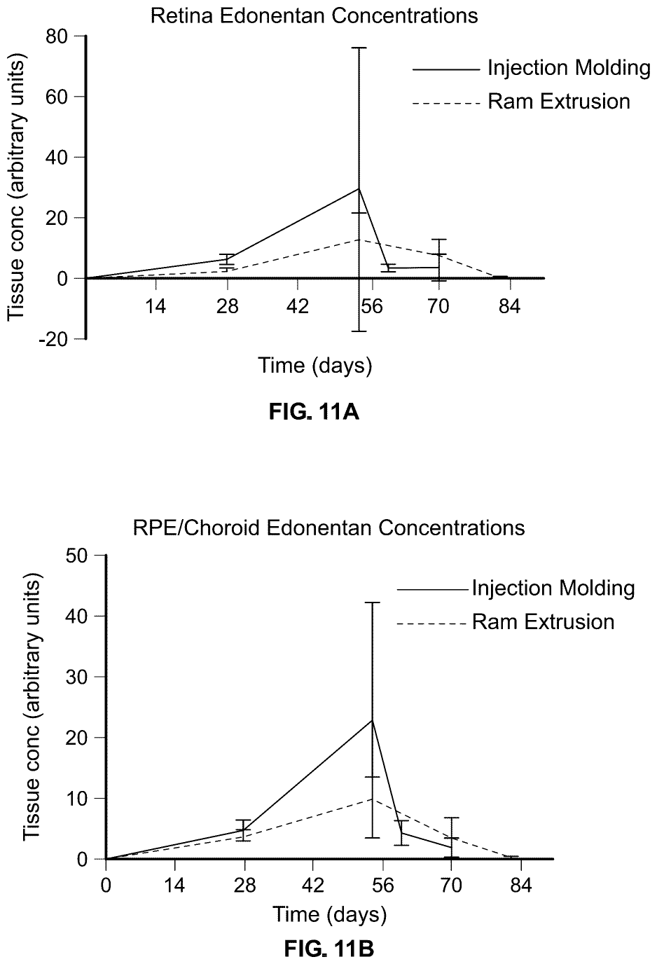

IVT sustained release delivery of 45% Edonentan in this PLGA implant demonstrated achievement of sustainable therapeutic target tissue levels of Edonentan for the duration of the study FIG. 11A, FIG. 11B. The cumulative total of Edonentan released from implants was 100% at 8 weeks as seen in Table 4 below.

TABLE 4
Cumulative Edonentan released from implants during
12-week ocular and systemic pharmacokinetic of
Edonentan intravitreal implant in rabbit study.

| | % Released | |
|---|---|---|
| | Injection Molded Edonentan | Ram Extruded Edonentan |
| Timepoint | Implants | Implants |
| Day 28 | 30.3 | 27.7 |
| Day 54 | 83.9 | 68.7 |
| Day 60 | 96.1 | NT |
| Day 70 | 96.0 | 94.5 |
| Day 82 | NT | 99.9 |

NT: not tested

### Example 15. Crystalline forms of Edonentan

Exemplary method of preparing crystalline Form 1

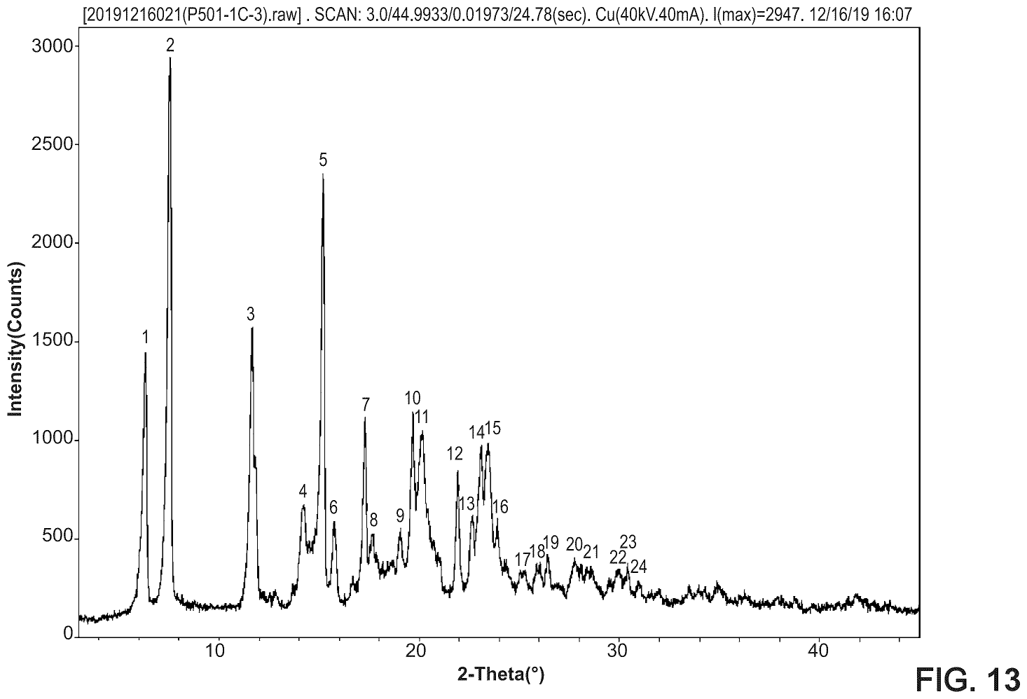

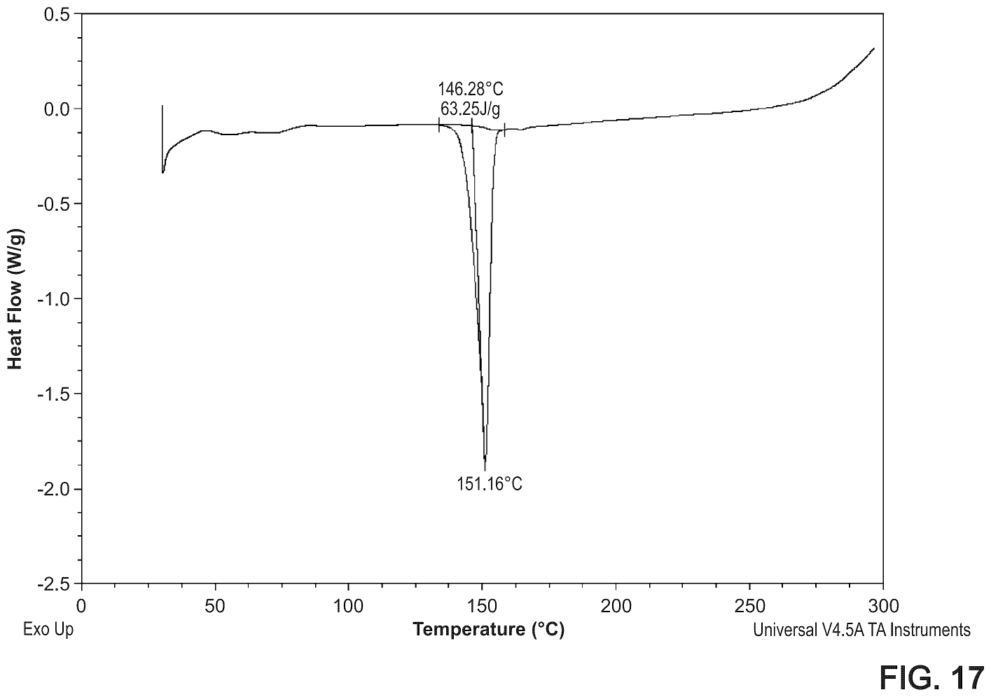

Amorphous Edonentan (840 mg) was dissolved in 12 mL of IPA. The resulting solution was filtered and the filter was washed with additional 2.5 mL of IPA. The filtrated was concentrated to dryness, dissolved in 11.8 mL of IPA and heated with stirring to 60° C. Then, 18 mL of warm water was added dropwise at 60° C. while stirring vigorously and the solution was stirred at 60° C. for 1 h. The solution was slowly cooled to 25° C., filtered and dried under vacuum at 25° C. to provide 660 mg of crystalline Form 1 (XRPD and DSC in FIG. 13 and FIG. 17, respectively).

Exemplary method of preparing crystalline Form 2

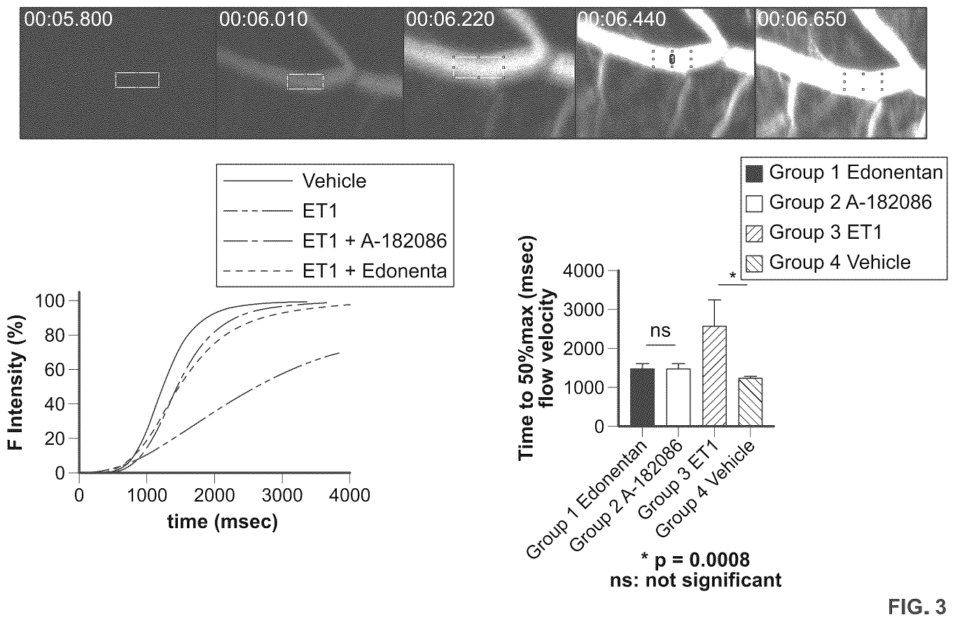

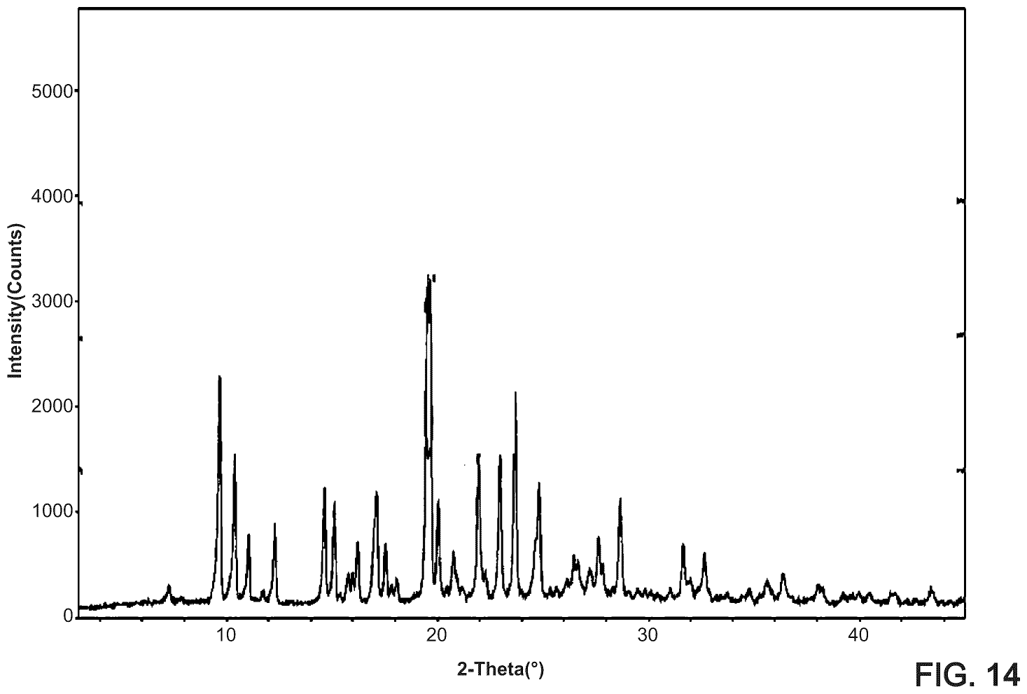

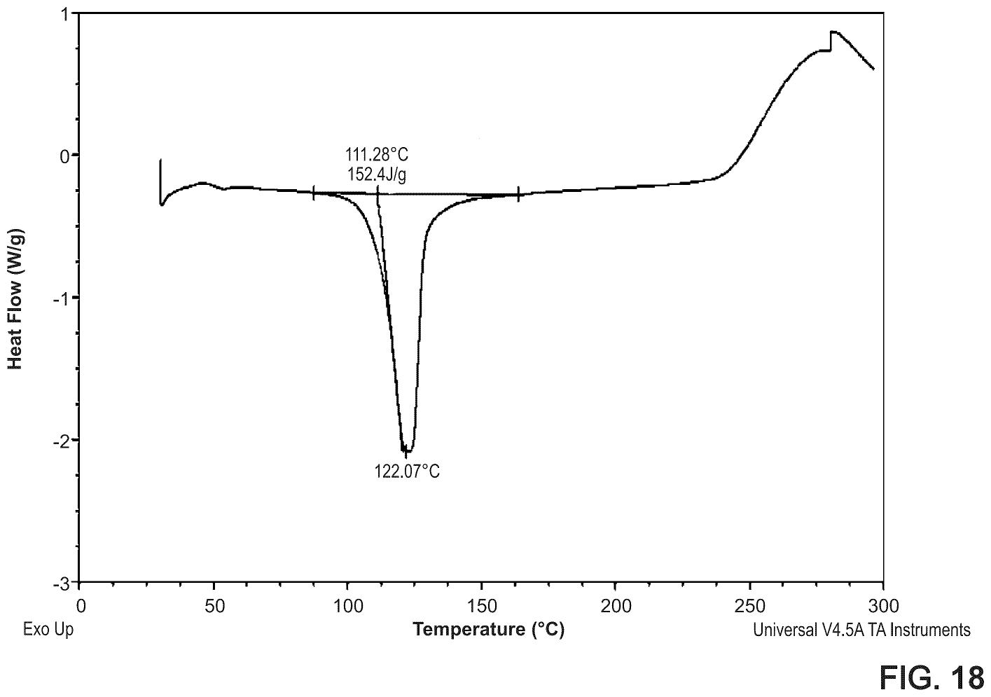

Amorphous Edonentan (250 mg) was dissolved in 3.5 mL of IPA. The resulting solution was filtered and the filter was washed with additional 0.25 mL of IPA. The solution was then heated to 60° C. whereupon 7.5 mL of warm water was added dropwise at 60° C. while stirring vigorously and then stirred at 60° C. for 1 h. After slowly cooling to 25° C., the mixture was filtered to provide crystalline Form 2 (XRPD and DSC in FIG. 3 and FIG. 7, respectively). Alternatively, a preferred method of preparing crystalline Form 2 is as follows. Amorphous Edonentan (1 g) was slurried in 20 mL of water at 25° C. for 15 hours. The solution was then filtered to give the crystalline Form 2 (XRPD and DSC in FIG. 14 and FIG. 18, respectively).

Exemplary method of preparing crystalline Form 3

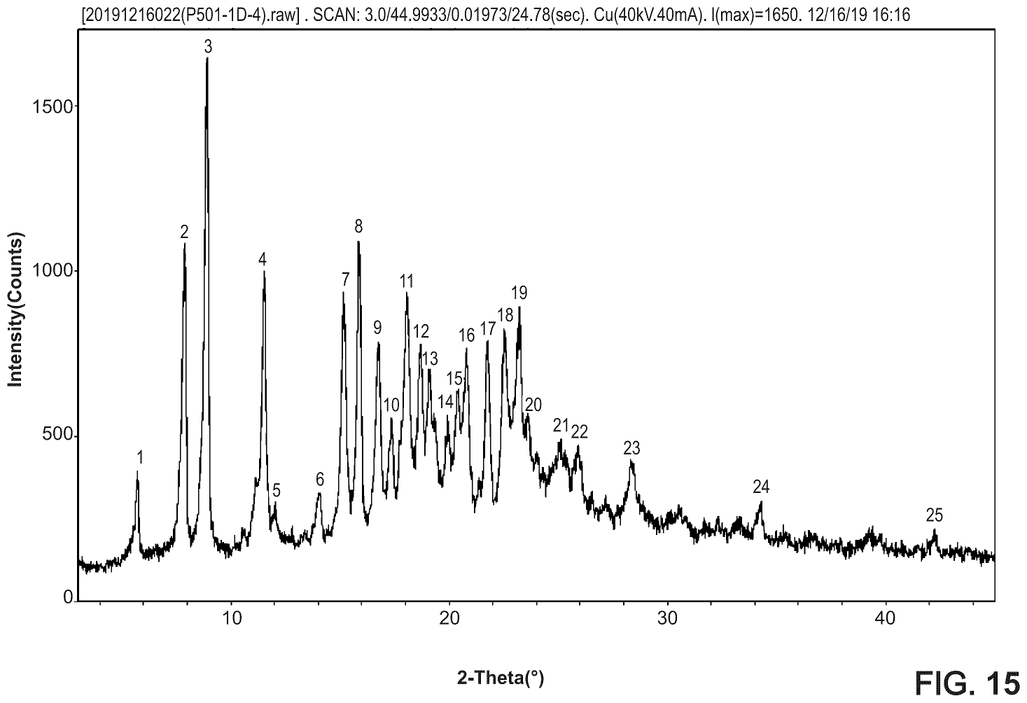

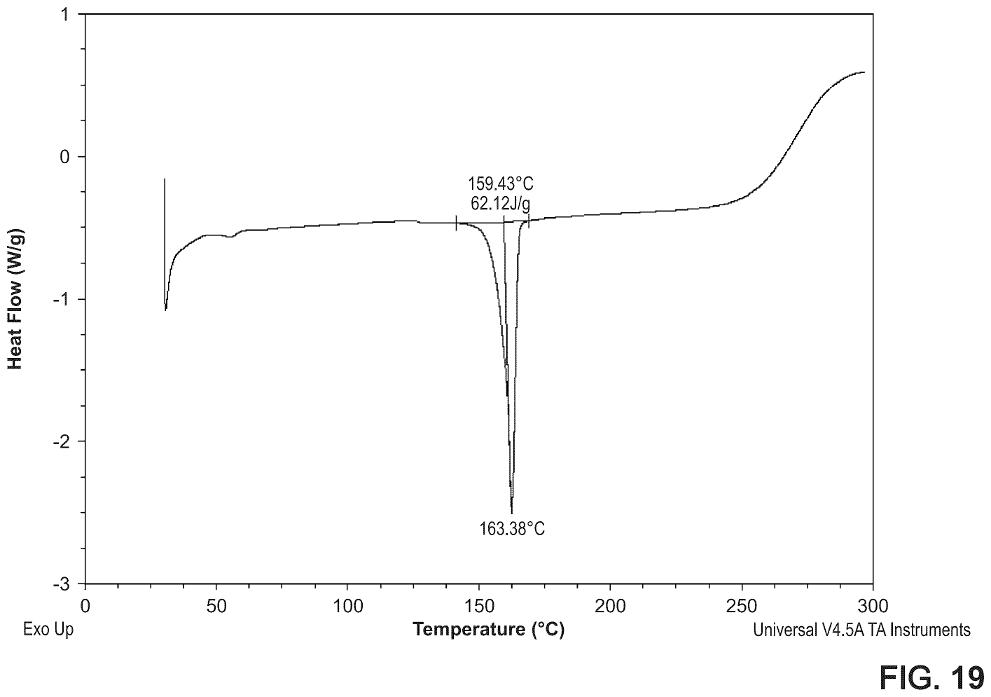

Amorphous Edonentan (250 mg) was dissolved in 0.5 mL of ethyl acetate. The resulting solution was filtered and heated to 60° C., and 1.5 mL of hexane was added dropwise at 60 ° C. while stirring vigorously. To the resulting slightly cloudy solution, 0.1 mL of ethyl acetate was added, resulting in a clear solution which was then stirred at 60° C. for 1 h. The solution was slowly cooled to 25° C. and the resulting precipitate was filtered to provide crystalline Form 3 (XRPD and DSC in FIG. 15 and FIG. 19, respectively).

Exemplary method of preparing crystalline Form 4

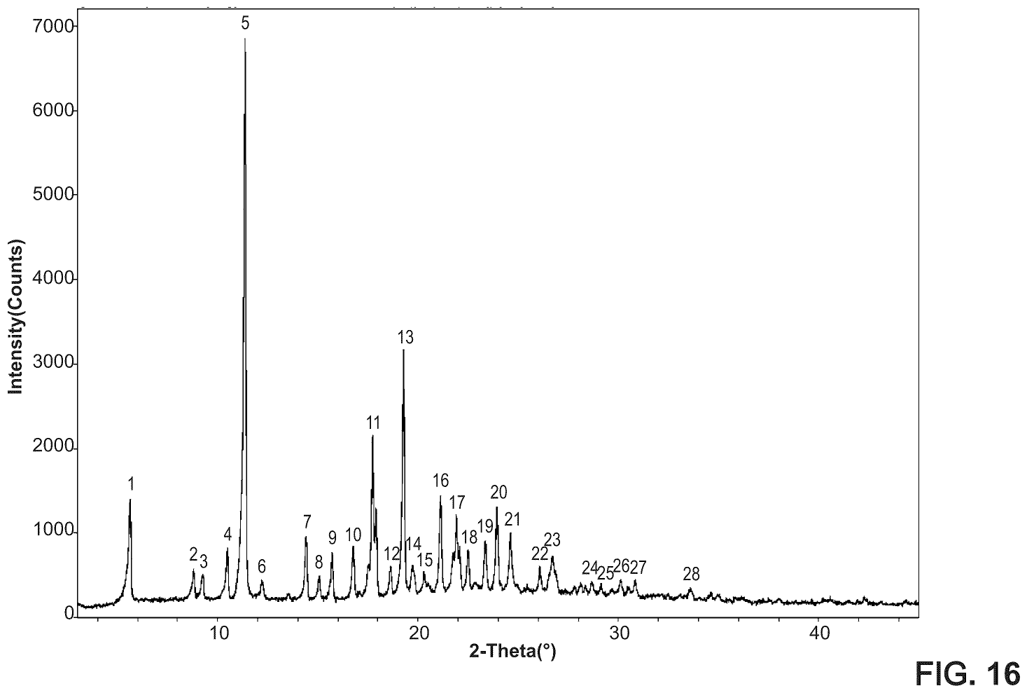

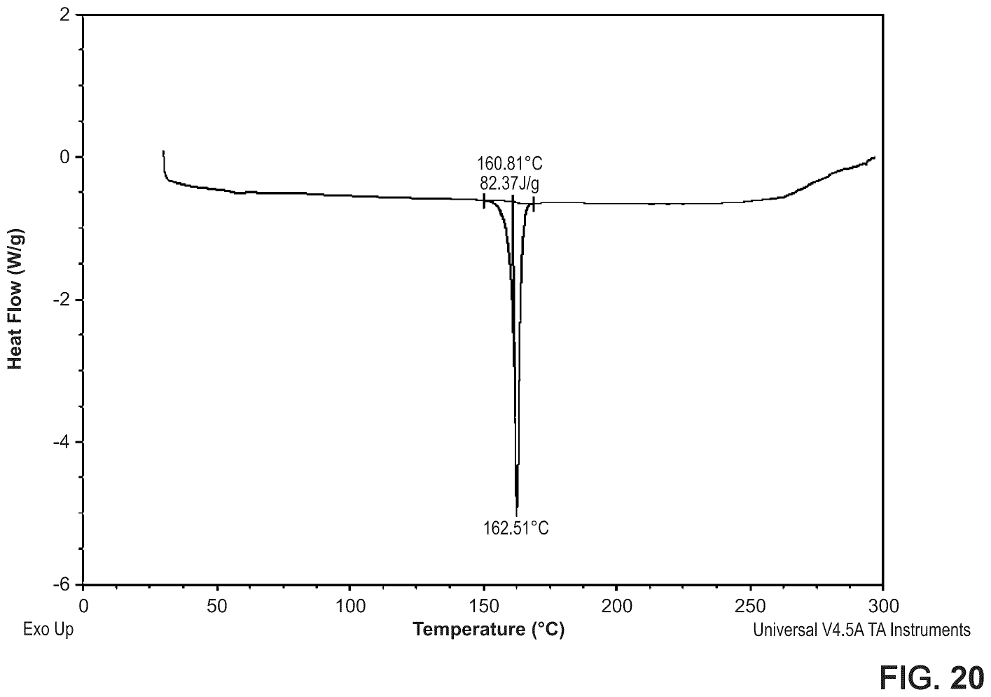

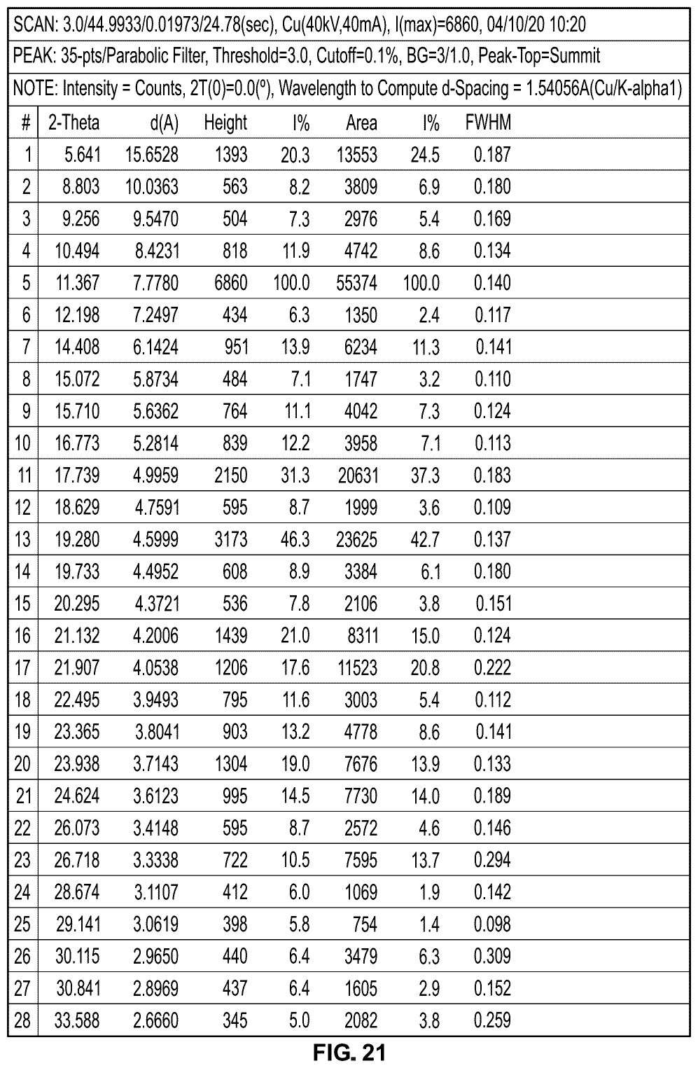

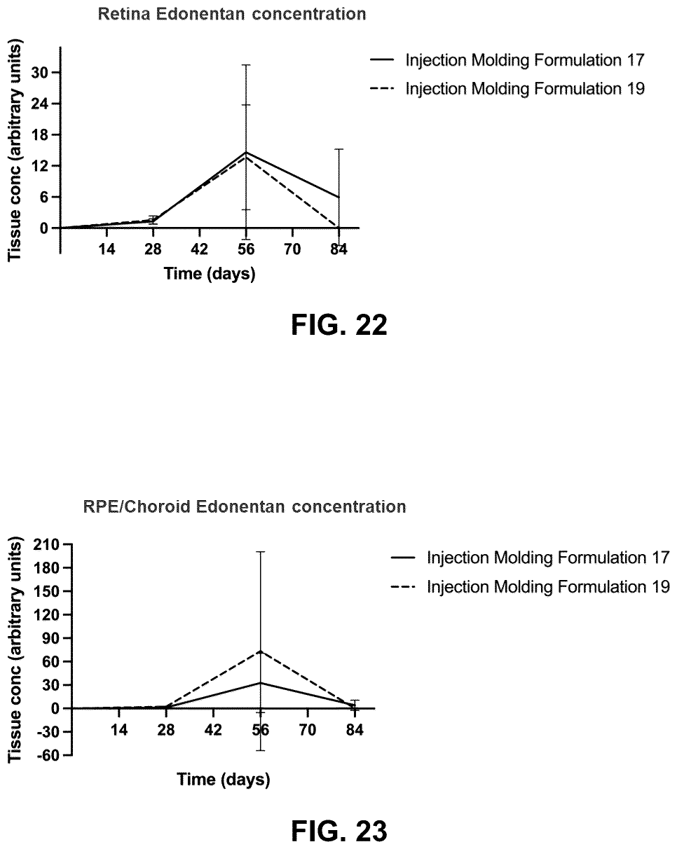

Amorphous Edonentan (100 mg) was added to 2 mL of water containing 0.2 mL of tetrahydrofuran (THF). The resulting mixture was stirred at 50° C. for 24 hours, cooled and filtered to provide Form 4, which was confirmed by XRPD (FIG. 16) and DSC (FIG. 20) to be distinct from Forms 1, 2 and 3.

In an alternate method, 107 mg of amorphous Edonentan was added to 1 mL of water followed by the addition of an equivalent of KOH in 1 mL of water. The resulting solution was heated to 60° C. for 20 minutes, filtered warm and acidified with 1 mL of 0.2 N HCl. The resulting mixture was stirred for 5 hours at 60° C., cooled and filtered to give Form 4, which was confirmed by XRPD.

In an alternate method, 150 mg of Edonentan (Form 3) was added to a mixture of isopropanol and water (1 mL and 2 mL, respectively). The resulting slurry was stirred at 15° C. for 48 hours and then filtered. The sample was confirmed by an XRPD analysis to be Form 4, demonstrating that under these conditions, Form 4 is more thermodynamically stable than Form 3.

In an alternate method, 200 mg of Edonentan (Form 1) was added to a mixture of isopropanol and water (1.3 mL and 2.6 mL, respectively). The resulting solution was heated to 80° C. and stirred for 24 h, then cooled and filtered. The sample thus obtained was confirmed by an XRPD analysis to be Form 4, demonstrating that under these conditions, Form 4 is more thermodynamically stable than Form 1.

In an alternate method, 100 mg of Edonentan (amorphous) was scurried in 10 mL of water and heated to 100° C. for 40 hours. The resulting solution was cooled to ambient temperature and filtered to afford Form 4. In an alternate method, amorphous (crude) Edonentan is dissolved in 8 volumes of isopropanol at 60° C. The resulting solution is cooled to 57° C., and then a small crystal of the crystalline Form 4 is added. After 2 hours, the solution is cooled to 5° C., held for 15 hours, and filtered to afford the crystalline Form 4.

### XRPD Patterns of Crystalline Forms

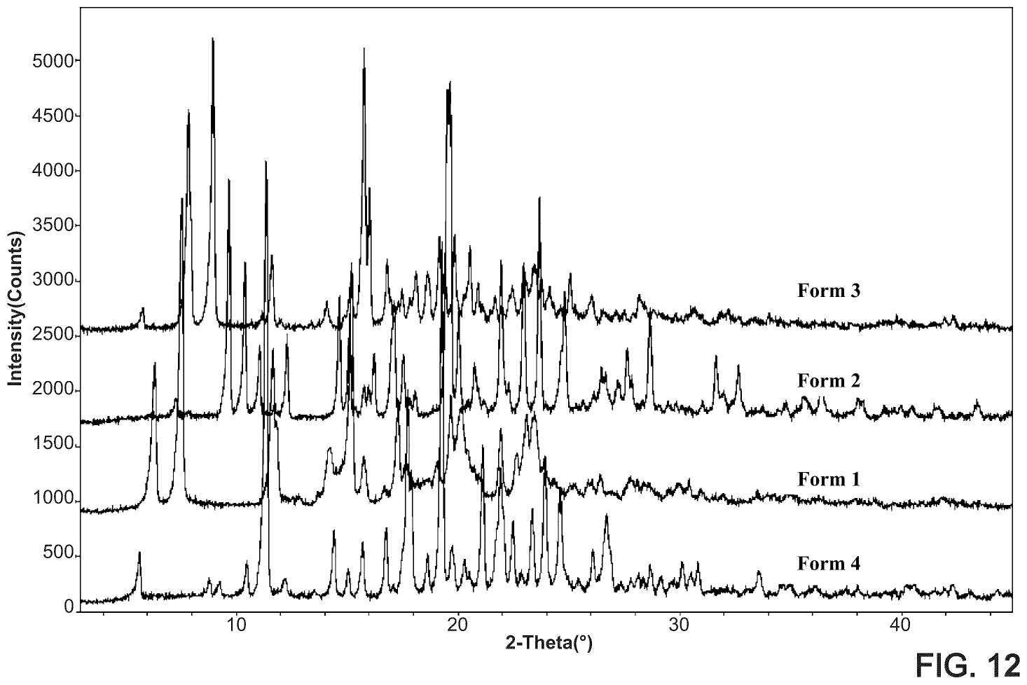

The XRPD patterns of crystalline Forms 1-4 are shown in FIGS. 12-16. The XRPD pattern of the crystalline form described herein was recorded using a Polycrystalline X-ray diffractometer (Bruker, D8 ADVANCE). The CuKa radiation was operating at a voltage of 40 kv and a current of 40 mA with a transmission slit of 1.0 mm and cable-stayed slit of 0.4°. A sample was placed in the center of sample holder groove and the surface of sample holder was leveled with the surface of sample holder. The data were collected over continuous scanning with a step size of 0.02° and a speed of 8°/min using the lynxeye detector.

The following Tables 5-8 list certain XRPD characteristic peaks for crystalline Forms 1-4, respectively.

TABLE 5
Exemplary XRPD patterns of crystalline Form 1

| 2θ | Intensity (counts) |
|---|---|
| 6.3 | 1250 |
| 7.5 | 2750 |
| 11.7 | 1400 |
| 15.1 | 2200 |
| 17.3 | 900 |

TABLE 6
Exemplary XRPD patterns of crystalline Form 2

| Angle [2θ] | Intensity (counts) |
|---|---|
| 9.6 | 2250 |
| 10.4 | 1500 |
| 11.1 | 600 |
| 12.3 | 750 |
| 14.6 | 1000 |
| 15.1 | 800 |
| 17.2 | 1000 |
| 19.6 | 3000 |
| 19.7 | 3000 |
| 22.0 | 1500 |
| 22.9 | 1500 |
| 23.7 | 2000 |

TABLE 7
Exemplary XRPD patterns of crystalline Form 3

| 2θ | Intensity (counts) |
|---|---|
| 7.8 | 2000 |
| 9.0 | 2750 |
| 11.6 | 750 |
| 15.8 | 2500 |
| 19.1 | 900 |

TABLE 8
Exemplary XRPD patterns of crystalline Form 4

| Angle [2θ] | Intensity (counts) |
|---|---|
| 5.6 | 1800 |
| 11.4 | 12600 |
| 14.4 | 1400 |
| 15.7 | 1200 |
| 16.8 | 1400 |
| 17.7 | 4800 |
| 19.3 | 6700 |
| 21.1 | 2900 |
| 21.9 | 2400 |
| 23.9 | 2400 |
| 24.6 | 1900 |

### Physiochemical Properties of Crystalline Forms

Provided herein are exemplary physicochemical properties of crystalline forms. The melting points described herein can be measured using the following procedure:
i. Melting Point Protocol

The maximal melting point peak (Tₘ) of each crystalline form was determined using DSC. The DSC of the crystalline form described herein was measured using the TA instrument DSC Q2000. A sample (1.3010 mg) was weighed in an aluminum crucible and heated from 30° C. to 300° C. at a heating rate of 10° C./min. Temperatures at crystalline melting peak start, peak onset, peak max, and peak end were collected.

The solubility described herein can be measured using the following procedure:
ii. Solubility Analysis Protocol
   1. No less than 2.0 mg samples are weighed into lower chamber of whatman miniuniprep vials (GE Healthcare). 450 μL of buffer was added into each chamber.
   2. Filter pistons of miniuniprep vials are placed and compressed to the position of the liquid level to allow for contact of buffer and compound with the filter during incubation.
   3. The samples are vortexed for 2 minutes followed by incubation at room temperature (about 25±2° C.) for 24 hours with shaking at 500 rpm.
   4. Miniunipreps are compressed to prepare the filtrates for injection into HPLC system. All vials are inspected for visible undissolved material before filtering and for leakage after filtering.
   5. Dilute supernatant with buffer by a factor of 100 folds to make diluents which are analyzed with HPLC.

Provided in Table 9 below are exemplary physicochemical properties of crystalline Forms 1-4. The physicochemical properties can be obtained using the methods described above.

TABLE 9
Exemplary physicochemical properties of crystalline Forms 1-4

| | | | Solubility |
|---|---|---|---|
| | | | pH 7.0 Phosphate Buffer |
| Polymorph | Solvation | Tₘ (° C.) | (μg/mL) |
| Form 1 | anhydrate | 151 | 264 |
| Form 2 | monohydrate | 122 | 35 |
| Form 3 | anhydrate | 162 | 251 |
| Form 4 | anhydrate | 163 | 138 |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further, from the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method for preventing, treating, or ameliorating an ocular neovascularization in a subject in need thereof, comprising contacting an optical tissue in a subject with a composition comprising a therapeutically effective amount of an endothelin receptor antagonist or a pharmaceutically acceptable salt thereof, wherein the endothelin receptor antagonist is selected from the group consisting of Edonentan, Tezosentan, A-182086, Clazosentan, 51255, ACT-132577, Enrasentan, and Sparsentan.

2. A method for preventing, treating, or ameliorating an ocular neovascularization in a subject in need thereof, comprising contacting an optical tissue in a subject with a composition comprising a therapeutically effective amount of either Edonentan or A-182086, or a pharmaceutically acceptable salt thereof.

3. A method for preventing, treating, or ameliorating an ocular neovascularization in a subject in need thereof, comprising contacting an optical tissue in a subject with a composition comprising a therapeutically effective amount of a compound of Formula I: 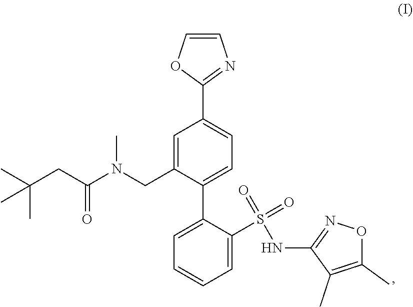 [SMILES: Cc1onc(NS(=O)(=O)c2ccccc2-c2ccc(-c3ncco3)cc2CN(C)C(=O)CC(C)(C)C)c1C]
or a pharmaceutically acceptable salt thereof.

4. The method of any one of claims 1-3, wherein the ocular neovascularization is associated with a condition selected from the group consisting of retinopathy of prematurity, retinal vein occlusion, macular edema, sickle cell retinopathy, choroidal neovascularization, radiation retinopathy, neovascular glaucoma, microangiopathy, retinal hypoxia, diabetic retinopathy, diabetic macular edema, ablation induced neovascularization, age related macular degeneration, and vascular leak.

5. The method of any one of claims 1-4, wherein therapeutic efficacy of the method is determined by the assessment of reduction in new vessel formation.

6. The method of any one of claims 1-5, wherein therapeutic efficacy of the method is determined by reduction in the rate of ocular neovascularization.

7. The method of any one of claims 1-6, wherein therapeutic efficacy of the treatment is indicated by an improvement in tissue or retinal perfusion.

8. The method of any one of claims 1-7, wherein therapeutic efficacy of the treatment is determined by assessing a degree of improvement in visual acuity, visual field, contrast sensitivity, or color vision.

9. The method of any one of claims 1-8, wherein the composition is administered in a dosage between about 1 μg and about 4 mg.

10. The method of any one of claims 1-9, wherein the composition is administered in a dosage between about 10 μg and about 500 μg.

11. The method of any one of claims 1-9, wherein the composition is administered in a dosage between about 150 μg and about 300 μg.

12. The method of any one of claims 1-9, wherein the composition is administered in a dosage between about 350 μg and about 500 μg.

13. The method of any one of claims 1-12, wherein the contacting comprises administering the composition topically to a surface of an eye or a portion thereof.

14. The method of any one of claims 1-12, wherein the contacting comprises injecting a composition into an eye or a component thereof.

15. The method of any one of claims 1-12, wherein the contacting comprises intravitreally administering the composition in a biodegradable ocular implant.

16. The method of any one of claims 1-15, wherein the composition comprises an ophthalmic preparation containing one or more preservatives, preservative aids, viscosity or lubrication adjusters, tonicity adjusters, solubilizers, buffers, surfactants, stabilizers, or a combination thereof.

17. The method of any one of claims 1-16, wherein the Edonentan or the compound of Formula I is an anhydrous crystalline form (Form 4), having an X-ray powder diffraction pattern comprising at least three characterization peaks, in terms of 2θ, selected from peaks at 5.6±0.2°, 11.4±0.2°, 17.7±0.2°, 19.3±0.2°, 21.1±0.2°, and 21.9±0.2°.

18. The method of claim 15, wherein the biodegradable ocular implant comprises a biodegradable polymer containing a compound incorporated therein; wherein the compound is a compound of Formula I or a pharmaceutically acceptable salt thereof, wherein the concentration of the compound in the biodegradable polymer is about 45% w/w; and the biodegradable polymer comprises RG503, RG502 and RG753S in a ratio of about 50%: about 10%: about 40%.

19. The method of claim 15, wherein the biodegradable ocular implant comprises a biodegradable polymer containing a compound incorporated therein; wherein the compound is a compound of Formula I or a pharmaceutically acceptable salt thereof, wherein the concentration of the compound in the biodegradable polymer is about 45% w/w; and the biodegradable polymer comprises RG503, RG502 and RG753S in a ratio of about 20%: about 20%: about 60%.

20. A method for preventing, treating, or ameliorating a vascular leakage in a subject in need thereof, comprising contacting an optical tissue in a subject with a composition comprising a therapeutically effective amount of an endothelin receptor antagonist or a pharmaceutically acceptable salt thereof, wherein the endothelin receptor antagonist is selected from the group consisting of Edonentan, Tezosentan, A-182086, Clazosentan, S1255, ACT-132577, Enrasentan, and Sparsentan.

21. A method for preventing, treating, or ameliorating a vascular leakage in a subject in need thereof, comprising contacting an optical tissue in a subject with a composition comprising a therapeutically effective amount of either Edonentan or A-182086, or a pharmaceutically acceptable salt thereof.

22. A method for preventing, treating, or ameliorating a vascular leakage in a subject in need thereof, comprising contacting an optical tissue in a subject with a composition comprising a therapeutically effective amount of a compound of Formula I: 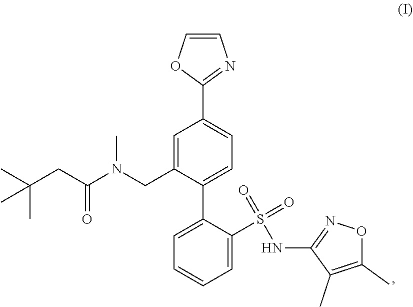 [SMILES: Cc1onc(NS(=O)(=O)c2ccccc2-c2ccc(-c3ncco3)cc2CN(C)C(=O)CC(C)(C)C)c1C]
or a pharmaceutically acceptable salt thereof.

23. The method of any one of claims 20-22, wherein the vascular leakage is associated with a condition selected from the group consisting of retinopathy of prematurity, retinal vein occlusion, macular edema, sickle cell retinopathy, choroidal neovascularization, radiation retinopathy, neovascular glaucoma, microangiopathy, retinal hypoxia, diabetic retinopathy, diabetic macular edema, ablation induced neovascularization, age related macular degeneration, and vascular leak.

24. The method of any one of claims 20-23, wherein therapeutic efficacy of the method is determined by reduction in the rate of vascular leakage.

25. The method of any one of claims 20-24, wherein therapeutic efficacy of the treatment is indicated by an improvement in tissue or retinal perfusion.

26. The method of any one of claims 20-25, wherein therapeutic efficacy of the treatment is determined by assessing a degree of improvement in visual acuity, visual field, contrast sensitivity, or color vision.

27. The method of any one of claims 20-26, wherein the composition is administered in a dosage between about 1 μg and about 4 mg.

28. The method of any one of claims 20-26, wherein the composition is administered in a dosage between about 10 μg and about 500 μg.

29. The method of any one of claims 20-26, wherein the composition is administered in a dosage between about 150 μg and about 300 μg.

30. The method of any one of claims 20-26, wherein the composition is administered in a dosage between about 350 μg and about 500 μg.

31. The method of any one of claims 20-30, wherein the contacting comprises administering the composition topically to a surface of an eye or a portion thereof.

32. The method of any one of claims 20-30, wherein the contacting comprises injecting a composition into an eye or a component thereof.

33. The method of any one of claims 20-30, wherein the contacting comprises intravitreally administering the composition in a biodegradable ocular implant.

34. The method of any one of claims 20-33, wherein the composition comprises an ophthalmic preparation containing one or more preservatives, preservative aids, viscosity or lubrication adjusters, tonicity adjusters, solubilizers, buffers, surfactants, stabilizers, or a combination thereof.

35. The method of any one of claims 20-34, wherein the Edonentan or the compound of Formula I is an anhydrous crystalline form (Form 4), having an X-ray powder diffraction pattern comprising at least three characterization peaks, in terms of 2θ, selected from peaks at 5.6±0.2°, 11.4±0.2°, 17.7±0.2°, 19.3±0.2°, 21.1±0.2°, and 21.9±0.2°.

36. The method of claim 33, wherein the biodegradable ocular implant comprises a biodegradable polymer containing a compound incorporated therein; wherein the compound is a compound of Formula I or a pharmaceutically acceptable salt thereof, wherein the concentration of the compound in the biodegradable polymer is about 45% w/w; and the biodegradable polymer comprises RG503, RG502 and RG753S in a ratio of about 50%: about 10%: about 40%.

37. The method of claim 33, wherein the biodegradable ocular implant comprises a biodegradable polymer containing a compound incorporated therein; wherein the compound is a compound of Formula I or a pharmaceutically acceptable salt thereof, wherein the concentration of the compound in the biodegradable polymer is about 45% w/w; and the biodegradable polymer comprises RG503, RG502 and RG753S in a ratio of about 20%: about 20%: about 60%.

38. A method for preventing, treating, or ameliorating a neovascular age-related macular degeneration in a subject in need thereof, comprising contacting an optical tissue in a subject in need thereof, comprising contacting an optical tissue in a subject with a composition comprising a therapeutically effective amount of an endothelin receptor antagonist or a pharmaceutically acceptable salt thereof, wherein the endothelin receptor antagonist is selected from the group consisting of Edonentan, Tezosentan, A-182086, Clazosentan, 51255, ACT-132577, Enrasentan, and Sparsentan.

39. A method for preventing, treating, or ameliorating a neovascular age-related macular degeneration in a subject in need thereof, comprising contacting an optical tissue in a subject with a composition comprising a therapeutically effective amount of either Edonentan or A-182086, or a pharmaceutically acceptable salt thereof.

40. A method for preventing, treating, or ameliorating a neovascular age-related macular degeneration in a subject in need thereof, comprising contacting an optical tissue in a subject with a composition comprising a therapeutically effective amount of a compound of Formula I: 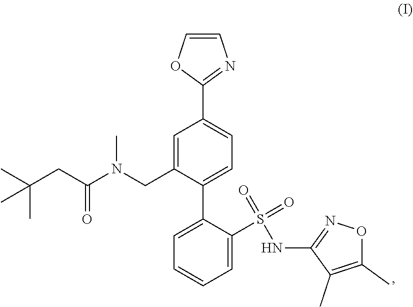 [SMILES: Cc1onc(NS(=O)(=O)c2ccccc2-c2ccc(-c3ncco3)cc2CN(C)C(=O)CC(C)(C)C)c1C]
or a pharmaceutically acceptable salt thereof.

41. The method of any one of claims 38-40, wherein therapeutic efficacy of the treatment is indicated by an improvement in tissue or retinal perfusion.

42. The method of any one of claims 38-41, wherein therapeutic efficacy of the treatment is determined by assessing a degree of improvement in visual acuity, visual field, contrast sensitivity, or color vision.

43. The method of any one of claims 38-42, wherein the composition is administered in a dosage between about 1 μg and about 4 mg.

44. The method of any one of claims 38-42, wherein the composition is administered in a dosage between about 10 μg and about 500 μg.

45. The method of any one of claims 38-42, wherein the composition is administered in a dosage between about 150 μg and about 300 μg.

46. The method of any one of claims 38-42, wherein the composition is administered in a dosage between about 350 μg and about 500 μg.

47. The method of any one of claims 38-46, wherein the contacting comprises administering the composition topically to a surface of an eye or a portion thereof.

48. The method of any one of claims 38-46, wherein the contacting comprises injecting a composition into an eye or a component thereof.

49. The method of any one of claims 38-46, wherein the contacting comprises intravitreally administering the composition in a biodegradable ocular implant.

50. The method of any one of claims 38-49, wherein the composition comprises an ophthalmic preparation containing one or more preservatives, preservative aids, viscosity or lubrication adjusters, tonicity adjusters, solubilizers, buffers, surfactants, stabilizers, or a combination thereof.

51. The method of any one of claims 38-50, wherein the Edonentan or the compound of Formula I is an anhydrous crystalline form (Form 4), having an X-ray powder diffraction pattern comprising at least three characterization peaks, in terms of 2θ, selected from peaks at 5.6±0.2°, 11.4±0.2°, 17.7±0.2°, 19.3±0.2°, 21.1±0.2°, and 21.9±0.2°.

52. The method of claim 49, wherein the biodegradable ocular implant comprises a biodegradable polymer containing a compound incorporated therein; wherein the compound is a compound of Formula I or a pharmaceutically acceptable salt thereof, wherein the concentration of the compound in the biodegradable polymer is about 45% w/w; and the biodegradable polymer comprises RG503, RG502 and RG753S in a ratio of about 50%: about 10%: about 40%.

53. The method of claim 49, wherein the biodegradable ocular implant comprises a biodegradable polymer containing a compound incorporated therein; wherein the compound is a compound of Formula I or a pharmaceutically acceptable salt thereof, wherein the concentration of the compound in the biodegradable polymer is about 45% w/w; and the biodegradable polymer comprises RG503, RG502 and RG753S in a ratio of about 20%: about 20%: about 60%.

54. A method for preventing, treating, or ameliorating a macular edema in a subject in need thereof, comprising contacting an optical tissue in a subject in need thereof, comprising contacting an optical tissue in a subject with a composition comprising a therapeutically effective amount of an endothelin receptor antagonist or a pharmaceutically acceptable salt thereof, wherein the endothelin receptor antagonist is selected from the group consisting of Edonentan, Tezosentan, A-182086, Clazosentan, 51255, ACT-132577, Enrasentan, and Sparsentan.

55. A method for preventing, treating, or ameliorating a macular edema in a subject in need thereof, comprising contacting an optical tissue in a subject with a composition comprising a therapeutically effective amount of either Edonentan or A-182086, or a pharmaceutically acceptable salt thereof.

56. A method for preventing, treating, or ameliorating a macular edema in a subject in need thereof, comprising contacting an optical tissue in a subject with a composition comprising a therapeutically effective amount of a compound of Formula I: 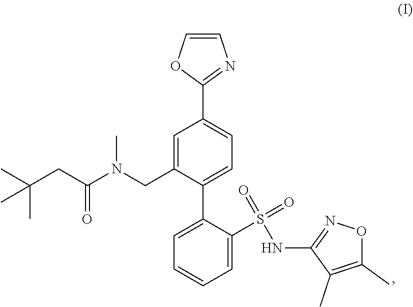 [SMILES: Cc1onc(NS(=O)(=O)c2ccccc2-c2ccc(-c3ncco3)cc2CN(C)C(=O)CC(C)(C)C)c1C]
or a pharmaceutically acceptable salt thereof.

57. The method of any one of claims 54-56, wherein therapeutic efficacy of the method is determined by the assessment of reduction in fluid in the macula.

58. The method of any one of claims 54-56, wherein therapeutic efficacy of the treatment is indicated by an improvement in tissue or retinal perfusion.

59. The method of any one of claims 54-58, wherein therapeutic efficacy of the treatment is determined by assessing a degree of improvement in visual acuity, visual field, contrast sensitivity, or color vision.

60. The method of any one of claims 54-59, wherein the composition is administered in a dosage between about 1 μg and about 4 mg.

61. The method of any one of claims 54-59, wherein the composition is administered in a dosage between about 10 μg and about 500 μg.

62. The method of any one of claims 54-59, wherein the composition is administered in a dosage between about 150 μg and about 300 μg.

63. The method of any one of claims 54-59, wherein the composition is administered in a dosage between about 350 μg and about 500 μg.

64. The method of any one of claims 54-63, wherein the contacting comprises administering the composition topically to a surface of an eye or a portion thereof.

65. The method of any one of claims 54-63, wherein the contacting comprises injecting a composition into an eye or a component thereof.

66. The method of any one of claims 54-63, wherein the contacting comprises intravitreally administering the composition in a biodegradable ocular implant.

67. The method of any one of claims 54-66, wherein the composition comprises an ophthalmic preparation containing one or more preservatives, preservative aids, viscosity or lubrication adjusters, tonicity adjusters, solubilizers, buffers, surfactants, stabilizers, or a combination thereof.

68. The method of any one of claims 54-67, wherein the Edonentan or the compound of Formula I is an anhydrous crystalline form (Form 4), having an X-ray powder diffraction pattern comprising at least three characterization peaks, in terms of 2θ, selected from peaks at 5.6±0.2°, 11.4±0.2°, 17.7±0.2°, 19.3±0.2°, 21.1±0.2°, and 21.9±0.2°.

69. The method of claim 66, wherein the biodegradable ocular implant comprises a biodegradable polymer containing a compound incorporated therein; wherein the compound is a compound of Formula I or a pharmaceutically acceptable salt thereof, wherein the concentration of the compound in the biodegradable polymer is about 45% w/w; and the biodegradable polymer comprises RG503, RG502 and RG753S in a ratio of about 50%: about 10%: about 40%.

70. The method of claim 66, wherein the biodegradable ocular implant comprises a biodegradable polymer containing a compound incorporated therein; wherein the compound is a compound of Formula I or a pharmaceutically acceptable salt thereof, wherein the concentration of the compound in the biodegradable polymer is about 45% w/w; and the biodegradable polymer comprises RG503, RG502 and RG753S in a ratio of about 20%: about 20%: about 60%.
